(19) United States
(12) Patent Application Publication
(10) Pub. No.: US 2011/0144076 A1
(43) Pub. Date: Jun. 16, 2011

(54) PRETERM DELIVERY DIAGNOSTIC ASSAY

(76) Inventors: Michelle A Williams, Seattle, WA (US); Daniel A. Enquobahrie, Brookine, MA (US)

(21) Appl. No.: 12/990,586

(22) PCT Filed: May 1, 2009
(86) PCT No.: PCT/US09/02730

§ 371 Date: Feb. 22, 2011

Related U.S. Application Data
(60) Provisional application No. 61/049,709, filed on May 1, 2008.

(51) Int. Cl. A61K 31/573; C12Q 1/68; C40B 30/00; C12Q 1/25; C40B 40/06; A61P 29/00; A61P 3/10; A61P 3/00; A61P 15/00

(52) U.S. Cl. 514/177; 435/6.12; 435/6.11; 506/7; 435/6.1; 435/29; 506/16

(57) ABSTRACT

The present invention in one aspect relates generally to the identification, provision and use of a plurality of biomarkers to provide risk assessment of a woman for preterm delivery, and products and processes related thereto. In one aspect, a novel plurality of biomarkers as described herein is provided to determine a risk for preterm delivery. In one aspect are methods for determining a risk of preterm delivery in a subject. In another aspect are methods of predicting the likelihood of preterm delivery in a subject. In yet another aspect are methods for identifying subjects at risk of preterm delivery, and kits for use in the method In yet another aspect are nucleic acid arrays comprising nucleic acid probes that hybridize to preterm delivery marker genes.

### CROSS REFERENCE TO RELATED APPLICATIONS

This application claims the benefit of U.S. Provisional Application No. 61/049,709, entitled, “Preterm Delivery Diagnostic Assay,” filed May 1, 2008, which is incorporated herein by reference in its entirety.

### STATEMENT AS TO FEDERALLY SPONSORED RESEARCH

This invention was made with the support of the United States government under Contract number 5 RO1 HD032562-10 by the National Institutes of Health, National Institute of Child Health and Human Development.

### BACKGROUND OF THE INVENTION

Preterm Delivery (PTD) is one of the most significant unsolved problems of public health and perinatology. Infants born preterm (<37 weeks gestation), as compared with infants born at term, are at greater risk for mortality and a wide range of medical and developmental complications. There is increasing evidence that PTD is a complex cluster of problems with a set of overlapping factors and influences. The causes of PTD include individual-level behavioral and psychological factors, environmental exposures, medical conditions, biological factors, and genetics, many of which occur in combination. To date, studies examining gene expression profiles in PTD have focused primarily on assessing profiles in tissue collected after delivery, prohibiting inferences concerning the temporal relation between altered gene expression profiles and onset of PTD. Failure to recognize important common pathophysiological pathways that may lead to PTD (e.g., systematic inflammation, endothelial dysfunction, oxidative stress, and placental ischemia) have hindered discovery of potential treatment and prevention strategies.

### SUMMARY OF THE INVENTION

Methods relating to determining a risk of preterm delivery in a subject are described herein. Methods of predicting the likelihood of preterm delivery in a subject are also described herein. Further described herein are methods for identifying subjects at risk of preterm delivery, and kits for use in the method. Further yet described herein are nucleic acid arrays comprising nucleic acid probes that hybridize to preterm delivery marker genes.

In one aspect of the invention are methods for determining a risk of preterm delivery in a subject, comprising: (i) comparing (a) a set of expression profiles of preterm delivery marker genes in a biological sample comprising peripheral blood cells from the subject to (b) a multimarker classifier; and (ii) providing a risk assessment for preterm delivery based on the comparison; wherein the set comprising expression profiles of a plurality of preterm delivery marker genes from Table 1, and the multimarker classifier was obtained by a comparison of expression levels of the preterm delivery marker genes in a plurality of women who delivered at term to expression levels of the preterm delivery marker genes in a plurality of women who delivered preterm.

In one embodiment of the methods for determining a risk of preterm delivery in a subject, the method further comprises obtaining the set of expression profiles prior to the comparing step.

In another embodiment of the methods for determining a risk of preterm delivery in a subject, the method further comprises obtaining or storing the biological sample prior to determining the set of expression profiles.

In another embodiment of the methods for determining a risk of preterm delivery in a subject, the obtaining the biological sample comprises isolating a mononuclear blood cell fraction from a whole blood sample from the subject.

In another embodiment of the methods for determining a risk of preterm delivery in a subject, the obtaining the biological sample comprises isolating lymphocytes from a whole blood sample from the subject.

In another embodiment of the methods for determining a risk of preterm delivery in a subject, the biological sample comprises a cell fraction enriched for mononuclear blood cells.

In another embodiment of the methods for determining a risk of preterm delivery in a subject, the cell fraction is enriched for lymphocytes.

In another embodiment of the methods for determining a risk of preterm delivery in a subject, the providing the risk assessment comprises providing a probability score.

In another embodiment of the methods for determining a risk of preterm delivery in a subject, the providing the risk assessment comprises providing a preterm delivery risk classification.

In another embodiment of the methods for determining a risk of preterm delivery in a subject, the preterm delivery is spontaneous preterm delivery.

In another embodiment of the methods for determining a risk of preterm delivery in a subject, the spontaneous preterm delivery is very preterm delivery, preterm premature rupture of membrane, moderate preterm delivery, or spontaneous preterm labor/delivery.

In another embodiment of the methods for determining a risk of preterm delivery in a subject, the plurality of preterm delivery marker genes comprises at least five of the preterm delivery marker genes listed in Table 2.

In another embodiment of the methods for determining a risk of preterm delivery in a subject, the plurality of preterm delivery marker genes comprises at least five of the preterm delivery marker genes listed in Table 4.

In another embodiment of the methods for determining a risk of preterm delivery in a subject, the plurality of preterm delivery marker genes comprises at least ten of the preterm delivery marker genes listed in Table 4.

In another embodiment of the methods for determining a risk of preterm delivery in a subject, the plurality of preterm delivery marker genes comprises the preterm delivery marker genes listed in Table 4.

In another embodiment of the methods for determining a risk of preterm delivery in a subject, the plurality of preterm delivery marker genes comprises at least ten of the preterm delivery marker genes listed in Table 3.

In another embodiment of the methods for determining a risk of preterm delivery in a subject, the plurality of preterm delivery marker genes comprises at least 30 of the preterm delivery marker genes listed in Table 3.

In another embodiment of the methods for determining a risk of preterm delivery in a subject, the plurality of preterm delivery marker genes comprises the preterm delivery marker genes listed in Table 3.

In another embodiment of the methods for determining a risk of preterm delivery in a subject, the risk assessment indicates that the subject has a high risk of preterm delivery, and further comprises prescribing or providing to the subject a prophylactic therapy for reducing the risk of preterm delivery.

In another embodiment of the methods for determining a risk of preterm delivery in a subject, the prophylactic therapy comprises progesterone therapy.

In another embodiment of the methods for determining a risk of preterm delivery in a subject, the prophylactic therapy comprises anti-inflammatory therapy.

In another embodiment of the methods for determining a risk of preterm delivery in a subject, the prophylactic therapy comprises anti-diabetic therapy.

In another embodiment of the methods for determining a risk of preterm delivery in a subject, the biological sample had been obtained antepartum at a gestational age no greater than 20 weeks.

In another embodiment of the methods for determining a risk of preterm delivery in a subject, the biological sample had been obtained at a gestational age from about 13 weeks to about 16 weeks.

In another embodiment of the methods for determining a risk of preterm delivery in a subject, the biological sample had been obtained within the first trimester of pregnancy.

In another aspect of the present invention are methods of predicting the likelihood of preterm delivery in a subject, comprising: (i) comparing expression profiles of a plurality of preterm delivery marker genes in a peripheral blood sample from the subject to: (a) expression profiles of the plurality of preterm delivery marker genes in peripheral blood samples from one or more subjects who delivered at term; or (b) expression profiles of the plurality of preterm delivery marker genes in blood samples from one or more subjects who delivered preterm; or (c) both (a) and (b); and (ii) providing a risk assessment based on the comparison; wherein the subject has an increased likelihood of preterm delivery if the expression profiles of the plurality of preterm deliver marker genes in the peripheral blood sample from the subject deviate from (a), and wherein the subject does not have an increased likelihood of preterm delivery if the expression profiles of the plurality of preterm delivery marker genes in the peripheral blood sample from the subject deviate from (b), and wherein the plurality of preterm delivery marker genes comprise five or more genes listed in Table 1.

In one embodiment of the methods of predicting the likelihood of preterm delivery in a subject, the method further comprises obtaining the gene expression profile prior to the comparing step.

In another embodiment of the methods of predicting the likelihood of preterm delivery in a subject, the method further comprises obtaining or storing the biological sample prior to determining the set of expression profiles.

In another embodiment of the methods of predicting the likelihood of preterm delivery in a subject, the obtaining the biological sample comprises isolating a mononuclear blood cell fraction from a whole blood sample from the subject.

In another embodiment of the methods of predicting the likelihood of preterm delivery in a subject, the obtaining the biological sample comprises isolating lymphocytes from a whole blood sample from the subject.

In another embodiment of the methods of predicting the likelihood of preterm delivery in a subject, the biological sample comprises a cell fraction enriched for mononuclear blood cells.

In another embodiment of the methods of predicting the likelihood of preterm delivery in a subject, the cell fraction is enriched for lymphocytes.

In some embodiments, determining expression profiles may be accomplished using an assay selected from the group consisting of a sequencing assay, a polymerase chain reaction assay, a hybridization assay, a hybridization assay employing a probe complementary to a mutation, fluorescent in situ hybridization, a nucleic acid array assay, a bead array assay, a primer extension assay, an enzyme mismatch cleavage assay, a branched hybridization assay, a NASBA assay, a molecular beacon assay, a cycling probe assay, a ligase chain reaction assay, an invasive cleavage structure assay, an ARMS assay, and a sandwich hybridization assay.

In another embodiment of the methods of predicting the likelihood of preterm delivery in a subject, the preterm delivery is spontaneous preterm delivery.

In another embodiment of the methods of predicting the likelihood of preterm delivery in a subject, the spontaneous preterm delivery is very preterm delivery, preterm premature rupture of membrane, moderate preterm delivery, or spontaneous preterm labor/delivery.

In yet another aspect of the present invention are methods for identifying a subject at risk of preterm delivery, comprising determining expression profiles of no more than five to five hundred genes in a biological sample comprising peripheral blood cells from a pregnant subject, wherein at least 20% of the genes are selected from the preterm delivery marker genes listed in Table 1.

In one embodiment of the methods for identifying a subject at risk of preterm delivery, at least 30% of the genes of the genes are selected from the preterm delivery marker genes listed in Table 1.

In another embodiment of the methods for identifying a subject at risk of preterm delivery, at least 30% of the genes are selected from the preterm delivery marker genes listed in Table 3.

In one embodiment of the methods for identifying a subject at risk of preterm delivery, at least 50% of the genes are selected from the preterm delivery marker genes listed in Table 3.

In one embodiment of the methods for identifying a subject at risk of preterm delivery, at least 90% of the genes are selected from the preterm delivery marker genes listed in Table 3.

In one embodiment of the methods for identifying a subject at risk of preterm delivery, the method comprises determining the expression profiles of no more than five to one hundred genes in a blood sample.

In one embodiment of the methods for identifying a subject at risk of preterm delivery, the method comprises determining expression profiles of no more than five to one hundred genes.

In one embodiment of the methods for identifying a subject at risk of preterm delivery, the method comprises determining expression profiles of no more than five to fifty genes.

In one embodiment of the methods for identifying a subject at risk of preterm delivery, the method comprises determining expression profiles of no more than five to twenty genes.

In one embodiment of the methods for identifying a subject at risk of preterm delivery, the method further comprises: (i) comparing the five to five hundred expression profiles to a multimarker classifier; and (ii) providing a risk assessment for preterm delivery based on the comparison; wherein the multimarker classifier was obtained by a comparison of expression levels of the preterm delivery marker genes in a plurality of women who delivered at term to expression levels of the preterm delivery marker genes in a plurality of women who delivered preterm.

In one embodiment of the methods for identifying a subject at risk of preterm delivery, the biological sample had been obtained antepartum at a gestational age no greater than 20 weeks.

In one embodiment of the methods for identifying a subject at risk of preterm delivery, the biological sample had been obtained at a gestational age from about 13 weeks to about 16 weeks.

In one embodiment of the methods for identifying a subject at risk of preterm delivery, the biological sample had been obtained within the first trimester of pregnancy.

In one embodiment of the methods for identifying a subject at risk of preterm delivery, the preterm delivery is spontaneous preterm delivery.

In one embodiment of the methods for identifying a subject at risk of preterm delivery, the spontaneous preterm delivery is very preterm delivery, preterm premature rupture of membrane, moderate preterm delivery, or spontaneous preterm labor/delivery.

In some embodiments, determining expression profiles may be accomplished using an assay selected from the group consisting of a sequencing assay, a polymerase chain reaction assay, a hybridization assay, a hybridization assay employing a probe complementary to a mutation, fluorescent in situ hybridization, a nucleic acid array assay, a bead array assay, a primer extension assay, an enzyme mismatch cleavage assay, a branched hybridization assay, a NASBA assay, a molecular beacon assay, a cycling probe assay, a ligase chain reaction assay, an invasive cleavage structure assay, an ARMS assay, and a sandwich hybridization assay.

In other embodiments, the methods can further include prescribing or providing to the subject a prophylactic therapy for reducing the risk of preterm delivery.

In one embodiment, the prophylactic therapy comprises progesterone therapy.

In another embodiment, the prophylactic therapy comprises anti-inflammatory therapy.

In another embodiment, the prophylactic therapy comprises anti-diabetic therapy.

In another embodiment, the prophylactic therapy comprises administering to said subject a therapy to reduce oxidative stress, intravascular hemolysis, endothelial dysfunction or a metabolic alteration associated with a high risk of preterm delivery.

In yet another aspect of the present invention are kits for use in the methods for identifying a subject at risk of preterm delivery, comprising: (i) a set of nucleic acid probes that hybridize under high stringency conditions to the nucleotide sequences of five to five hundred genes in a biological sample comprising peripheral blood cells from a pregnant subject, wherein at least 20% of the genes are selected from the preterm delivery marker genes listed in Table 1, for determining the expression profiles of said genes; and an insert describing: (a) an expression profile of one or more of the preterm delivery marker genes in blood samples from one or more subjects who delivered at term; (b) an expression profile of one or more preterm delivery marker genes in blood samples from one or more subjects who delivered preterm; or (c) a multimarker classifier, wherein the multimarker classifier was obtained by a comparison of expression levels of the preterm delivery marker genes in a plurality of women who delivered at term to expression levels of the preterm delivery marker genes in a plurality of women who delivered preterm.

In one embodiment of the kits, the set of nucleic acid probes comprise primers for RT-PCR amplification of the mRNAs for the ten to one thousand preterm delivery marker genes.

In yet another aspect of the present invention are nucleic acid arrays comprising nucleic acid probes that hybridize under high stringency conditions to the nucleotide sequences of no more than five to five hundred genes, wherein at least 20% of the genes are selected from the preterm delivery marker genes listed in Table 1.

In one embodiment of the nucleic acid arrays, the nucleic acid array is provided as one or more multiwell plates, comprising primers for RT-PCR amplification of the mRNAs for the ten to one thousand preterm delivery marker genes.

In another embodiment of the nucleic acid arrays, the nucleic acid array is provided as a nucleic acid hybridization microarray.

In another embodiment of the nucleic acid arrays, at least 30% of the genes of the genes are selected from the preterm delivery marker genes listed in Table 1.

In another embodiment of the nucleic acid arrays, at least 30% of the genes of the genes are selected from the preterm delivery marker genes listed in Table 3.

In another embodiment of the nucleic acid arrays, at least 50% of the genes of the genes are selected from the preterm delivery marker genes listed in Table 3.

In another embodiment of the nucleic acid arrays, at least 90% of the genes of the genes are selected from the preterm delivery marker genes listed in Table 3.

In another embodiment of the nucleic acid arrays, the array comprises nucleic acid probes that hybridize under high stringency conditions to the nucleotide sequences of no more than five to one hundred genes.

In another embodiment of the nucleic acid arrays, the array comprises nucleic acid probes that hybridize under high stringency conditions to the nucleotide sequences of no more than five to fifty genes.

In another embodiment of the nucleic acid arrays, the array comprises nucleic acid probes that hybridize under high stringency conditions to the nucleotide sequences of no more than five to twenty genes.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an illustrative volcano plot of placental gene expression data.

FIG. 2 is an illustrative Students' T-test P-value and SAM false discovery rate.

FIG. 3 is an illustrative Venn diagram summary of distribution of differentially expressed genes.

FIG. 4 is an illustrative heat map illustration of phylogenetic tree of samples and selected differentially selected genes.

FIG. 5 is an illustrative graph of pathway networks identified using Ingenuity Path Analysis.

FIG. 6 is an illustrative graph of PCA results from 69 genes.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention in one aspect relates generally to the identification, provision and use of a plurality of biomarkers to provide risk assessment of a woman for preterm delivery, and products and processes related thereto. In one aspect, a novel plurality of biomarkers as described herein is provided to determine a risk for preterm delivery. In one aspect are methods for determining a risk of preterm delivery in a subject. In another aspect are methods of predicting the likelihood of preterm delivery in a subject. In yet another aspect are methods for identifying subjects at risk of preterm delivery, and kits for use in the method. In yet another aspect are nucleic acid arrays comprising nucleic acid probes that hybridize to preterm delivery marker genes.

Some embodiments of the invention allow for inferences concerning the temporal relation between altered gene expression profiles and onset of PTD. Further, gene expression profiles from antepartum whole blood samples can reflect gene expression in leukocytes and provide biologically relevant samples that can be obtained with minimal risk and discomfort.

As used herein, “preterm delivery” or PTD means delivery that occurs before 37 weeks gestation, and includes spontaneous preterm delivery and medically induced preterm delivery. Spontaneous preterm delivery (sPTD) means spontaneous delivery 20 to <36 weeks gestation. Subgroups of spontaneous preterm delivery include, but are not limited to, very preterm delivery (VPTD, 20-<33 weeks gestation); moderate preterm delivery (MPTD, 33-<36 weeks gestation); spontaneous preterm labor/delivery (sPTL, clinical presentation of SPTD), and spontaneous preterm premature rupture of membranes (PPROM).

Further as used herein, a “biomarker” is an indicator of a particular disease state or state of a subject. As a non-limiting example, the biomarker is a gene.

### Expression Profiles of Preterm Delivery

Without limiting the scope of the present invention, any number of techniques known in the art can be employed for expression profiling of preterm delivery biomarkers.

In some embodiments, the detecting step(s) comprises use of a detection assay including, but not limited to, sequencing assays, polymerase chain reaction assays, hybridization assays, hybridization assay employing a probe complementary to a mutation, fluorescent in situ hybridization (FISH), nucleic acid array assays, bead array assays, primer extension assays, enzyme mismatch cleavage assays, branched hybridization assays, NASBA assays, molecular beacon assays, cycling probe assays, ligase chain reaction assays, invasive cleavage structure assays, ARMS assays, and sandwich hybridization assays. In some preferred embodiments, the detecting step is carried out using cell lysates. In some embodiments, the methods may comprise detecting a second nucleic acid target. In some preferred embodiments, the second nucleic acid target is RNA. In some particularly preferred embodiments, the second nucleic acid target may be, for example, U6 RNA or GAPDH mRNA.

In one embodiment, one of skill in the art can choose to detect genes that exhibit a fold increase above background of at least 2. In another embodiment, one of skill in the art can choose to detect genes that exhibited a fold increase or decrease above background of at least 3, and in another embodiment at least 4, and in another embodiment at least 5, and in another embodiment at least 6, and in another embodiment at least 7, and in another embodiment at least 8, and in another embodiment at least 9, and in another embodiment at least 10 or higher fold changes. It is noted that fold increases or decreases are not typically compared from one gene to another, but with reference to the background level for that particular gene.

In one aspect of the method of the present invention, the expression profile can include the expression of one or more of the genes disclosed herein. Expression of transcripts is measured by any of a variety of known methods in the art.

For RNA expression, methods include but are not limited to: extraction of cellular mRNA and Northern blotting using labeled probes that hybridize to transcripts encoding all or part of one or more of the genes of this invention; amplification of mRNA expressed from one or more of the genes of this invention using gene-specific primers, polymerase chain reaction (PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene.

In addition to general expression of a gene, the number of copies of a gene in a cell can be determined with nucleic acid probes to the genes. In one embodiment, Fluorescent in situ hybridization (FISH) can be used to detect the number of copies of a gene in a cell. Established hybridization techniques such as FISH are contemplated herein. In one embodiment, the number of genes within a peripheral blood cell are detected using a FISH assay for a plurality of preterm delivery markers disclosed herein.

Nucleic acid arrays are particularly useful for detecting the expression of the genes of the present invention. The production and application of high-density arrays in gene expression monitoring have been disclosed previously in, for example, WO 97/10365; WO 92/10588; U.S. Pat. No. 6,040,138; U.S. Pat. No. 5,445,934; or WO95/35505, all of which are incorporated herein by reference in their entireties. Also for examples of arrays, see Hacia et al. (1996) Nature Genetics 14:441-447; Lockhart et al. (1996) Nature Biotechnol. 14:1675-1680; and De Risi et al. (1996) Nature Genetics 14:457-460. In general, in an array, an oligonucleotide, a cDNA, or genomic DNA, that is a portion of a known gene, occupies a known location on a substrate. A nucleic acid target sample is hybridized with an array of such oligonucleotides and then the amount of target nucleic acids hybridized to each probe in the array is quantified. One preferred quantifying method is to use confocal microscope and fluorescent labels. The Affymetrix GeneChip™ Array system (Affymetrix, Santa Clara, Calif.) and the Atlas™ Human cDNA Expression Array system are particularly suitable for quantifying the hybridization; however, it will be apparent to those of skill in the art that any similar systems or other effectively equivalent detection methods can also be used. In a particularly preferred embodiment, one can use the knowledge of the genes described herein to design novel arrays of polynucleotides, cDNAs or genomic DNAs for screening methods described herein. Such novel pluralities of polynucleotides are contemplated to be a part of the present invention and are described in detail below.

Suitable nucleic acid samples for screening on an array contain transcripts of interest or nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like. Preferably, the nucleic acids for screening are obtained from a homogenate of cells or tissues or other biological samples. Preferably, such sample is a total RNA preparation of a biological sample. More preferably in some embodiments, such a nucleic acid sample is the total mRNA isolated from a biological sample.

In one embodiment, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids to achieve quantitative amplification. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high-density array may then include probes specific to the internal standard for quantification of the amplified nucleic acid. Other suitable amplification methods include, but are not limited to, polymerase chain reaction (PCR) (see Innis, et al., PCR Protocols, A Guide to Methods and Application, Academic Press, Inc. San Diego, (1990)); ligase chain reaction (LCR) (see Wu and Wallace, Genomics, 4: 560 (1989), Landegren, et al., Science, 241:1077 (1988) and Barringer, et al., Gene, 89:117 (1990)); transcription amplification (Kwoh, et al., Proc. Natl. Acad. Sci. USA, 86:1173 (1989)), and self-sustained sequence replication (Guatelli, et al., Proc. Nat. Acad. Sci. USA, 87:1874 (1990)).

Nucleic acid hybridization simply involves contacting a probe and target nucleic acid under conditions where the probe and its complementary target can form stable hybrid duplexes through complementary base pairing. As used herein, hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Laboratory Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., Ibid., all of which are incorporated by reference herein in their entirety. Nucleic acids that do not form hybrid duplexes are washed away from the hybridized nucleic acids and the hybridized nucleic acids can then be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

High stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). One of skill in the art can use the formulae in Meinkoth et al., 1984, Anal. Biochem. 138, 267-284 (incorporated herein by reference in its entirety) to calculate the appropriate hybridization and wash conditions to achieve these particular levels of nucleotide mismatch. Such conditions will vary, depending on whether DNA.-RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na+) at a temperature of between about 20° C. and about 35° C., more preferably, between about 28° C. and about 40° C., and even more preferably, between about 35° C. and about 45° C. In particular embodiments, stringent hybridization conditions for DNA:RNA hybrids include hybridization at an ionic strength of 6×SSC (0.9 M Na+) at a temperature of between about 30° C. and about 45° C., more preferably, between about 38° C. and about 50° C., and even more preferably, between about 45° C. and about 55° C. These values are based on calculations of a melting temperature for molecules larger than about 100 nucleotides, 0% formamide and a G+C content of about 40%. Alternatively, Tm can be calculated empirically as set forth in Sambrook et al., supra, pages 9.31 to 9.62.

The hybridized nucleic acids are detected by detecting one or more labels attached to the sample nucleic acids. The labels may be incorporated by any of a number of means well known to those of skill in the art. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., 3H, ¹²⁵I, ³⁵S, ¹⁴C or ³²P), enzymes (e.g., horseradish peroxidase, alkaline phosphatase and others commonly used in an ELISA), and colorimetric labels such as colloidal gold or colored glass or plastic (e.g., polystyrene, polypropylene, latex, etc.) beads. Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

In some embodiments of the present invention, detection structures are detected using a hybridization assay. In a hybridization assay, the presence of absence of a given nucleic acid sequence is determined based on the ability of the DNA from the sample to hybridize to a complementary DNA molecule (e.g., an oligonucleotide probe). A variety of hybridization assays using a variety of technologies for hybridization and detection are available and include, but are not limited, to those described herein.

The complement of a nucleic acid sequence as used herein refers to an oligonucleotide which, when aligned with the nucleic acid sequence such that the 5′ end of one sequence is paired with the 3′ end of the other, is in “anti-parallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

In some embodiments, hybridization of a probe to the sequence of interest (e.g., a SNP or mutation) is detected directly by visualizing a bound probe (e.g., a Northern or Southern assay; see e.g., Ausabel et al. (eds.), Current Protocols in Molecular Biology, John Wiley & Sons, NY [1991]). In these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the SNP or mutation being detected is allowed to contact the membrane under a condition of low, medium, or high stringency conditions. Unbound probe is removed and the presence of binding is detected by visualizing the labeled probe.

For analysis by Northern blotting, total RNA isolation is performed by acid guanidinium thiocyanate-phenol-chloroform extraction. Northern analysis is performed as described according to standard protocols, except that the total RNA is resolved on a 15% denaturing polyacrylamide gel, transferred onto Hybond-N⁺ membrane (Amersham Pharmacia Biotech), and the hybridization and wash steps are performed at 50 ° C. Oligodeoxynucleotides used as Northern probes are 5′-³²P-phosphorylated, complementary to the miRNA sequence and 20 to 25 nt in length. 5S rRNA is detected by ethidium staining of polyacrylamide gels prior to transfer. Blots are stripped by boiling in 0.1% aqueous sodium dodecylsulfate/0.1×SSC (15 mM sodium chloride, 1.5 mM sodium citrate, pH 7.0) for 10 min, and are re-probed up to 4 times until the 21-nt signals become too weak for detection. Finally, blots are probed for val-tRNA as size marker.

In some embodiments of the present invention, variant sequences are detected using a DNA chip hybridization assay. In this assay, a series of oligonucleotide probes are affixed to a solid support. The oligonucleotide probes are designed to be unique to a given target sequence (e.g., miRNA target sequence). The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) may be utilized (see e.g., U.S. Pat. Nos. 6,017,696; 6,068,818; and 6,051,380; each of which is herein incorporated by reference). Through the use of microelectronics, Nanogen's technology enables the active movement and concentration of charged molecules to and from designated test sites on its semiconductor microchip. DNA capture probes unique to a given target sequence are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target sequences by determining which of the DNA capture probes hybridize, with target sequences. An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding.

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on an x/Y translation stage under a set of four piezoelectric nozzles, one for each of the four standard DNA bases. The translation stage moves along each of the rows of the array and the appropriate reagent is delivered to each of the reaction sites. For example, the A amidite is delivered only to the sites where amidite A is to be coupled during that synthesis step and so on. Common reagents and washes are delivered by flooding the entire surface and then removing them by spinning.

DNA probes unique for the target sequence (e.g., miRNA target sequence) of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a bead array technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the bead array is contacted with a prepared subject sample (e.g., nucleic acid sample). Hybridization is detected using any suitable method.

In some embodiments of the present invention, hybridization is detected by enzymatic cleavage of specific structures.

In some embodiments, hybridization of a bound probe is detected using a TaqMan® assay (PE Biosystems, Foster City, Calif.; See e.g., U.S. Pat. Nos. 5,962,233 and 5,538,848, each of which is herein incorporated by reference). The assay is performed during a PCR reaction. The TaqMan® assay exploits the 5′-3′ exonuclease activity of the AMPLITAQ GOLD® DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD® polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the target sequence location. Incorporation of the label into the DNA can be detected by any suitable method (e.g., if the nucleotide contains a biotin label, detection is via a fluorescently labeled antibody specific for biotin).

Additional detection assays useful in the detection of miRNA detection structures include, but are not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Barnay, PNAS USA 88, 189-93 (1991)); and sandwich hybridization methods (e.g., U.S. Pat. No. 5,288,609, herein incorporated by reference in its entirety).

The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

### Multimarker Classifier

In one aspect of the invention, multimarker classifiers can be utilized. In one embodiment, the multimarker classifier is obtained by a comparison of expression levels of genes in a plurality of women who delivered at term to expression levels of genes in a plurality of women who delivered preterm, and identifying genes that were statistically significantly differentially expressed between the two pluralities.

In one embodiment of the invention, the multimarker classifier comprises a plurality or all of the 611 preterm delivery genes identified in Table 1. In another embodiment of the invention, the multimarker classifier comprises a plurality or all of the 253 preterm delivery genes identified in Table 2 (all 253 of which are found in the list of 611 genes). In yet another embodiment, the multimarker classifier comprises a plurality or all of the 69 genes identified in Table 3 (all 69 of which are found in the lists of 253 and 611 genes). In a further embodiment of the invention, the multimarker classifier comprises a plurality or all of the 27 genes identified in Table 4 (all 27 of which are found in the lists of 69, 253 and 611 genes). The genes in Tables 1-4 are genes which have the potential to discriminate between women who will go on to deliver preterm versus those who will deliver at term. In certain embodiments of the invention, a plurality of genes selected from the 27 genes identified in Table 4 are used with the products and methods described and claimed herein to discriminate between women who will go on to deliver preterm versus those who will deliver at term. In some embodiments of the invention, a plurality of genes selected from the 27 genes identified in Table 4 are used with the products and methods described and claimed herein to determine a risk of, or predict the likelihood of, preterm delivery. In certain embodiments of the invention, a plurality of genes selected from the 69 genes identified in Table 3 are used with the products and methods described and claimed herein to discriminate between women who will go on to deliver preterm versus those who will deliver at term. In some embodiments of the invention, a plurality of genes selected from the 69 genes identified in Table 3 are used with the products and methods described and claimed herein to determine a risk of, or predict the likelihood of, preterm delivery. In certain embodiments of the invention, a plurality of genes selected from the 253 genes identified in Table 2 are used with the products and methods described and claimed herein to discriminate between women who will go on to deliver preterm versus those who will deliver at term. In some embodiments of the invention, a plurality of genes selected from the 253 genes identified in Table 2 are used with the products and methods described and claimed herein to determine a risk of, or predict the likelihood of, preterm delivery. In certain embodiments of the invention, a plurality of genes selected from the 611 genes identified in Table 1 are used with the products and methods described and claimed herein to discriminate between women who will go on to deliver preterm versus those who will deliver at term. In some embodiments of the invention, a plurality of genes selected from the 611 genes identified in Table 1 are used with the products and methods described and claimed herein to determine a risk of, or predict the likelihood of, preterm delivery.

TABLE 1
611 Genes for Preterm Delivery

| Affy | | t-test | | Control | Preterm | Control | Preterm | | | | | | |
|---|---|---|---|---|---|---|---|---|---|---|---|---|---|
| Probe | Fold | P- | T- | avg | avg | (norm) | (norm) | Log | Log | P-value | | Sequence | |
| IDs | Change | value | statistic | Intensity | Intensity | Intensity | Intensity | (Ratio) | (Error) | Resolver | Accession # | Name | Sequence Description |
| 237695_at | 8.18 | 0.0151 | −2.75 | 1.56 | 12.73 | 0.01 | 0.06 | 0.91 | 0.53 | 0.0426 | BF197664 | LOC442421 | similar to prostaglandin E |
| | | | | | | | | | | | | | receptor 4, subtype EP4; |
| | | | | | | | | | | | | | PGE receptor, EP4 |
| | | | | | | | | | | | | | subtype; prostaglandin E2 |
| | | | | | | | | | | | | | receptor |
| 1566548_at | 5.24 | 0.0407 | −2.15 | 2.43 | 12.72 | 0.01 | 0.06 | 0.72 | 0.14 | 0.0000 | AL049918 | DHRSX | Dehydrogenase/reductase |
| | | | | | | | | | | | | | (SDR family) X-linked |
| 232897_at | 4.08 | 0.0494 | −2.14 | 3.74 | 15.25 | 0.02 | 0.07 | 0.61 | 0.30 | 0.0342 | AK000451 | FLJ20444 | hypothetical protein |
| | | | | | | | | | | | | | FLJ20444 |
| 215666_at | 3.54 | 0.0689 | −1.92 | 18.31 | 64.73 | 0.08 | 0.30 | 0.55 | 0.44 | 0.1720 | U70544 | HLA-DRB4 | Major histocompatibility |
| | | | | | | | | | | | | | complex, class II, DR beta 1 |
| 205838_at | 3.53 | 0.2853 | −1.10 | 2.63 | 9.31 | 0.01 | 0.04 | 0.55 | 0.50 | 0.2654 | NM_002099 | GYPA | Glycophorin A (MNS |
| | | | | | | | | | | | | | blood group) |
| 1554663_a_at | 3.31 | 0.0607 | −2.00 | 10.48 | 34.69 | 0.05 | 0.16 | 0.52 | 0.29 | 0.0670 | BC043499 | NUMA1 | Nuclear mitotic apparatus |
| | | | | | | | | | | | | | protein 1 |
| 220281_at | 3.15 | 0.3573 | −0.95 | 3.99 | 12.58 | 0.02 | 0.06 | 0.50 | 0.43 | 0.2485 | AI632015 | SLC12A1 | Solute carrier family 12 |
| | | | | | | | | | | | | | (sodium/potassium/chloride |
| | | | | | | | | | | | | | transporters), member 1 |
| 239586_at | 3.00 | 0.3017 | −1.06 | 4.82 | 14.48 | 0.02 | 0.07 | 0.48 | 0.45 | 0.2937 | AA085776 | FAM83A | Family with sequence |
| | | | | | | | | | | | | | similarity 83, member A |
| 226147_s_at | 3.00 | 0.2093 | −1.32 | 7.99 | 23.97 | 0.04 | 0.11 | 0.48 | 0.48 | 0.3275 | AA838075 | PIGR | Polymeric |
| | | | | | | | | | | | | | immunoglobulin receptor |
| 206778_at | 2.97 | 0.0013 | −3.79 | 5.84 | 17.36 | 0.03 | 0.08 | 0.47 | 0.15 | 0.0016 | NM_000496 | CRYBB2 | Crystallin, beta B2 |
| 241420_at | 2.81 | 0.1298 | −1.59 | 37.75 | 106.03 | 0.17 | 0.49 | 0.45 | 0.29 | 0.1226 | BF057027 | LOC653138 | similar to similar to |
| | | | | | | | | | | | | | RPL23AP7 protein |
| 1553296_at | 2.77 | 0.2451 | −1.21 | 4.04 | 11.19 | 0.02 | 0.05 | 0.44 | 0.40 | 0.2819 | NM_032787 | GPR128 | G protein-coupled receptor |
| | | | | | | | | | | | | | 128 |
| 205826_at | 2.75 | 0.1990 | −1.33 | 93.05 | 256.02 | 0.43 | 1.19 | 0.44 | 0.27 | 0.1033 | NM_003970 | MYOM2 | Myomesin (M-protein) 2, |
| | | | | | | | | | | | | | 165 kDa |
| 220161_s_at | 2.73 | 0.1456 | −1.51 | 7.52 | 20.56 | 0.03 | 0.10 | 0.44 | 0.25 | 0.0707 | NM_019114 | EPB41L4B | Erythrocyte membrane |
| | | | | | | | | | | | | | protein band 4.1 like 4B |
| 221642_at | 2.72 | 0.5506 | −0.61 | 19.00 | 51.69 | 0.09 | 0.24 | 0.43 | 0.50 | 0.3952 | BC002903 | TREX1 | Homo sapiens three prime |
| | | | | | | | | | | | | | repair exonuclease 1, |
| | | | | | | | | | | | | | mRNA (cDNA clone |
| | | | | | | | | | | | | | IMAGE: 3944613), partial |
| | | | | | | | | | | | | | cds. |
| 241841_at | 2.72 | 0.1624 | −1.47 | 4.22 | 11.46 | 0.02 | 0.05 | 0.43 | 0.32 | 0.1813 | AI298755 | CPT1B | Carnitine |
| | | | | | | | | | | | | | palmitoyltransferase 1B |
| | | | | | | | | | | | | | (muscle) |
| 229622_at | 2.59 | 0.1810 | −1.38 | 7.38 | 19.09 | 0.03 | 0.09 | 0.41 | 0.32 | 0.2070 | H16258 | FLJ37034 | hypothetical protein |
| | | | | | | | | | | | | | FLJ37034 |
| 237962_x_at | 2.41 | 0.3961 | −0.86 | 7.82 | 18.84 | 0.04 | 0.09 | 0.38 | 0.16 | 0.0136 | BF912264 | KIAA1267 | KIAA1267 |
| 244319_at | 2.31 | 0.0782 | −1.85 | 11.22 | 25.91 | 0.05 | 0.12 | 0.36 | 0.21 | 0.0842 | AW770718 | DDEF2 | Development and |
| | | | | | | | | | | | | | differentiation enhancing |
| | | | | | | | | | | | | | factor 2 |
| 243898_at | 2.30 | 0.0654 | −1.93 | 3.36 | 7.72 | 0.02 | 0.04 | 0.36 | 0.16 | 0.0243 | AA699656 | RHBDD1 | Rhomboid domain |
| | | | | | | | | | | | | | containing 1 |
| 209728_at | 2.27 | 0.1320 | −1.55 | 355.46 | 806.86 | 1.64 | 3.75 | 0.36 | 0.30 | 0.2234 | BC005312 | HLA-DRB4 | Major histocompatibility |
| | | | | | | | | | | | | | complex, class II, DR beta 1 |
| 211613_s_at | 2.23 | 0.0051 | −3.06 | 5.46 | 12.18 | 0.03 | 0.06 | 0.35 | 0.14 | 0.0111 | U79250 | GPD2 | Glycerol-3-phosphate |
| | | | | | | | | | | | | | dehydrogenase 2 |
| | | | | | | | | | | | | | (mitochondrial) |
| 219955_at | 2.23 | 0.3102 | −1.04 | 11.47 | 25.52 | 0.05 | 0.12 | 0.35 | 0.39 | 0.3831 | NM_019079 | L1TD1 | LINE-1 type transposase |
| | | | | | | | | | | | | | domain containing 1 |
| 203498_at | 2.21 | 0.2166 | −1.27 | 6.21 | 13.75 | 0.03 | 0.06 | 0.35 | 0.22 | 0.1150 | NM_005822 | DSCR1L1 | Down syndrome critical |
| | | | | | | | | | | | | | region gene 1-like 1 |
| 241024_at | 2.20 | 0.2408 | −1.20 | 4.05 | 8.92 | 0.02 | 0.04 | 0.34 | 0.22 | 0.1180 | AI493466 | C6orf148 | Chromosome 6 open |
| | | | | | | | | | | | | | reading frame 148 |
| 206777_s_at | 2.20 | 0.0267 | −2.47 | 60.29 | 132.88 | 0.28 | 0.62 | 0.34 | 0.12 | 0.0066 | NM_000496 | CRYBB2 | Crystallin, beta B2 |
| 219054_at | 2.19 | 0.0920 | −1.75 | 3.55 | 7.78 | 0.02 | 0.04 | 0.34 | 0.17 | 0.0459 | NM_024563 | C5orf23 | Chromosome 5 open |
| | | | | | | | | | | | | | reading frame 23 |
| 217603_at | 2.19 | 0.0825 | −1.83 | 7.45 | 16.34 | 0.03 | 0.08 | 0.34 | 0.12 | 0.0050 | AW444520 | ATP6V0A2 | ATPase, H+ transporting, |
| | | | | | | | | | | | | | lysosomal V0 subunit a2 |
| 1566889_at | 2.19 | 0.0176 | −2.60 | 14.56 | 31.82 | 0.07 | 0.15 | 0.34 | 0.13 | 0.0118 | BC037847 | THADA | Thyroid adenoma |
| | | | | | | | | | | | | | associated |
| 206856_at | 2.16 | 0.0234 | −2.47 | 5.33 | 11.55 | 0.02 | 0.05 | 0.34 | 0.14 | 0.0184 | NM_006840 | LILRB5 | Leukocyte |
| | | | | | | | | | | | | | immunoglobulin-like |
| | | | | | | | | | | | | | receptor, subfamily B |
| | | | | | | | | | | | | | (with TM and ITIM |
| | | | | | | | | | | | | | domains), member 2 |
| 230787_at | 2.16 | 0.2916 | −1.08 | 7.45 | 16.06 | 0.03 | 0.07 | 0.33 | 0.19 | 0.0750 | AW197616 | 230787_at | Transcribed locus |
| 227326_at | 2.13 | 0.1879 | −1.35 | 29.98 | 63.86 | 0.14 | 0.30 | 0.33 | 0.20 | 0.1089 | BE966768 | MXRA7 | 601661268R1 |
| | | | | | | | | | | | | | NIH_MGC_72 Homo |
| | | | | | | | | | | | | | sapiens cDNA clone |
| | | | | | | | | | | | | | IMAGE: 3916097 3′, |
| | | | | | | | | | | | | | mRNA sequence. |
| 231484_at | 2.11 | 0.1990 | −1.34 | 21.85 | 46.00 | 0.10 | 0.21 | 0.32 | 0.23 | 0.1748 | AI424825 | ATP8A1 | ATPase, |
| | | | | | | | | | | | | | aminophospholipid |
| | | | | | | | | | | | | | transporter (APLT), Class |
| | | | | | | | | | | | | | I, type 8A, member 1 |
| 207891_s_at | 2.10 | 0.0018 | −3.46 | 3.78 | 7.96 | 0.02 | 0.04 | 0.32 | 0.11 | 0.0035 | NM_017518 | UIP1 | 26S proteasome-associated |
| | | | | | | | | | | | | | UCH interacting protein 1 |
| 205654_at | 2.10 | 0.5055 | −0.68 | 64.78 | 136.25 | 0.30 | 0.63 | 0.32 | 0.39 | 0.4003 | NM_000715 | C4BPA | Complement component 4 |
| | | | | | | | | | | | | | binding protein, alpha |
| 237009_at | 2.10 | 0.0088 | −2.82 | 8.48 | 17.82 | 0.04 | 0.08 | 0.32 | 0.15 | 0.0311 | BF439675 | CD69 | CD69 molecule |
| 1552950_at | 2.10 | 0.1658 | −1.43 | 4.24 | 8.88 | 0.02 | 0.04 | 0.32 | 0.23 | 0.1689 | NM_173528 | C15orf26 | Chromosome 15 open |
| | | | | | | | | | | | | | reading frame 26 |
| 206632_s_at | 2.08 | 0.3319 | −0.99 | 177.02 | 367.80 | 0.82 | 1.71 | 0.32 | 0.32 | 0.3139 | NM_004900 | APOBEC3B | Apolipoprotein B mRNA |
| | | | | | | | | | | | | | editing enzyme, catalytic |
| | | | | | | | | | | | | | polypeptide-like 3B |
| 205138_s_at | 2.05 | 0.0045 | −3.11 | 4.21 | 8.62 | 0.02 | 0.04 | 0.31 | 0.14 | 0.0187 | AW418882 | UST | Uronyl-2-sulfotransferase |
| 231313_at | 2.04 | 0.0038 | −3.25 | 3.75 | 7.65 | 0.02 | 0.04 | 0.31 | 0.13 | 0.0160 | AW134984 | LRRC8B | Leucine rich repeat |
| | | | | | | | | | | | | | containing 8 family, |
| | | | | | | | | | | | | | member B |
| 244774_at | 2.02 | 0.1181 | −1.65 | 11.84 | 23.97 | 0.05 | 0.11 | 0.31 | 0.21 | 0.1548 | R81072 | PHACTR2 | Phosphatase and actin |
| | | | | | | | | | | | | | regulator 2 |
| 241762_at | 2.02 | 0.1508 | −1.50 | 11.74 | 23.70 | 0.05 | 0.11 | 0.31 | 0.18 | 0.0870 | BF244402 | FBXO32 | 601862985F1 |
| | | | | | | | | | | | | | NIH_MGC_57 Homo |
| | | | | | | | | | | | | | sapiens cDNA clone |
| | | | | | | | | | | | | | IMAGE: 4080500 5′, |
| | | | | | | | | | | | | | mRNA sequence. |
| 217520_x_at | 2.01 | 0.0699 | −1.88 | 3.74 | 7.50 | 0.02 | 0.03 | 0.30 | 0.16 | 0.0553 | BG396614 | LOC646278 | similar to programmed cell |
| | | | | | | | | | | | | | death 6 interacting protein |
| 244629_s_at | 2.01 | 0.0921 | −1.78 | 7.70 | 15.45 | 0.04 | 0.07 | 0.30 | 0.23 | 0.2005 | BE670141 | PDPK1 | 3-phosphoinositide |
| | | | | | | | | | | | | | dependent protein kinase-1 |
| 1557610_at | 2.01 | 0.0048 | −3.11 | 5.60 | 11.23 | 0.03 | 0.05 | 0.30 | 0.11 | 0.0051 | AI003930 | PITRM1 | Pitrilysin metallopeptidase 1 |
| 214043_at | 1.98 | 0.3203 | −1.02 | 4.61 | 9.14 | 0.02 | 0.04 | 0.30 | 0.23 | 0.1936 | BF062299 | PTPRD | Protein tyrosine |
| | | | | | | | | | | | | | phosphatase, receptor type, D |
| 211343_s_at | 1.98 | 0.8282 | −0.22 | 18.27 | 36.10 | 0.08 | 0.17 | 0.30 | 0.39 | 0.4615 | M33653 | COL13A1 | Collagen, type XIII, alpha 1 |
| 205048_s_at | 1.97 | 0.2016 | −1.32 | 72.44 | 142.88 | 0.33 | 0.66 | 0.30 | 0.20 | 0.1438 | NM_004577 | PSPH | Phosphoserine phosphatase |
| 1554984_a_at | 1.97 | 0.0181 | −2.61 | 5.78 | 11.39 | 0.03 | 0.05 | 0.29 | 0.18 | 0.1163 | BC020226 | HLA-DOB | Major histocompatibility |
| | | | | | | | | | | | | | complex, class II, DO beta |
| 214238_at | 1.95 | 0.0289 | −2.31 | 6.46 | 12.57 | 0.03 | 0.06 | 0.29 | 0.12 | 0.0127 | AI093572 | 214238_at | Clone DT1P1B6 mRNA, |
| | | | | | | | | | | | | | CAG repeat region |
| 238954_at | 1.95 | 0.1924 | −1.35 | 7.98 | 15.53 | 0.04 | 0.07 | 0.29 | 0.27 | 0.2978 | AW851069 | 238954_at | Transcribed locus, weakly |
| | | | | | | | | | | | | | similar to XP_516162.1 |
| | | | | | | | | | | | | | PREDICTED: hypothetical |
| | | | | | | | | | | | | | protein XP_516162 [Pantroglodytes] |
| 243990_at | 1.93 | 0.0612 | −1.95 | 3.89 | 7.51 | 0.02 | 0.03 | 0.29 | 0.17 | 0.0870 | AI861840 | CRYL1 | Crystallin, lambda 1 |
| 1560359_at | 1.91 | 0.0217 | −2.46 | 4.19 | 8.00 | 0.02 | 0.04 | 0.28 | 0.13 | 0.0372 | BG619261 | PELO | Pelota homolog |
| | | | | | | | | | | | | | (Drosophila) |
| 217381_s_at | 1.90 | 0.0861 | −1.78 | 5.15 | 9.79 | 0.02 | 0.05 | 0.28 | 0.17 | 0.0953 | X69383 | TRGV5 | H. sapiens gene for T cell |
| | | | | | | | | | | | | | receptor gamma V region |
| | | | | | | | | | | | | | 5. |
| 1555177_at | 1.90 | 0.0344 | −2.29 | 12.88 | 24.48 | 0.06 | 0.11 | 0.28 | 0.19 | 0.1637 | BC012622 | PRKAA1 | Protein kinase, AMP- |
| | | | | | | | | | | | | | activated, alpha 1 catalytic |
| | | | | | | | | | | | | | subunit |
| 1554140_at | 1.88 | 0.2176 | −1.27 | 4.62 | 8.67 | 0.02 | 0.04 | 0.27 | 0.28 | 0.3325 | BC032406 | WDR78 | WD repeat domain 78 |
| 240496_at | 1.88 | 0.0188 | −2.51 | 6.49 | 12.17 | 0.03 | 0.06 | 0.27 | 0.23 | 0.2018 | AI652000 | KCNH2 | Potassium voltage-gated |
| | | | | | | | | | | | | | channel, subfamily H (eag- |
| | | | | | | | | | | | | | related), member 2 |
| 229168_at | 1.87 | 0.0165 | −2.55 | 5.43 | 10.14 | 0.03 | 0.05 | 0.27 | 0.13 | 0.0458 | AI690433 | COL23A1 | Collagen, type XXIII, |
| | | | | | | | | | | | | | alpha 1 |
| 212741_at | 1.86 | 0.2957 | −1.08 | 8.74 | 16.25 | 0.04 | 0.08 | 0.27 | 0.27 | 0.3323 | AA923354 | MAOA | Monoamine oxidase A |
| 227989_at | 1.85 | 0.0897 | −1.76 | 3.79 | 7.01 | 0.02 | 0.03 | 0.27 | 0.14 | 0.0506 | AI927486 | LTBP4 | Latent transforming |
| | | | | | | | | | | | | | growth factor beta binding |
| | | | | | | | | | | | | | protein 4 |
| 206145_at | 1.85 | 0.4673 | −0.74 | 8.06 | 14.87 | 0.04 | 0.07 | 0.27 | 0.29 | 0.3732 | NM_000324 | RHAG | Rh-associated glycoprotein |
| 236687_at | 1.85 | 0.0258 | −2.39 | 4.82 | 8.90 | 0.02 | 0.04 | 0.27 | 0.13 | 0.0355 | BG150083 | TIRAP | Toll-interleukin 1 receptor |
| | | | | | | | | | | | | | (TIR) domain containing |
| | | | | | | | | | | | | | adaptor protein |
| 227955_s_at | 1.84 | 0.5231 | −0.65 | 5.39 | 9.89 | 0.02 | 0.05 | 0.26 | 0.27 | 0.3348 | BE670307 | 227955_s_at | CDNA: FLJ22256 fis, |
| | | | | | | | | | | | | | clone HRC02860 |
| 217623_at | 1.83 | 0.0523 | −2.05 | 6.18 | 11.32 | 0.03 | 0.05 | 0.26 | 0.16 | 0.1134 | BF114815 | 217623_at | Transcribed locus, |
| | | | | | | | | | | | | | moderately similar to |
| | | | | | | | | | | | | | XP_517655.1 |
| | | | | | | | | | | | | | PREDICTED: similar to |
| | | | | | | | | | | | | | KIAA0825 protein [Pantroglodytes] |
| 200884_at | 1.83 | 0.0368 | −2.20 | 4.48 | 8.19 | 0.02 | 0.04 | 0.26 | 0.16 | 0.0946 | NM_001823 | CKB | Creatine kinase, brain |
| 218623_at | 1.83 | 0.0956 | −1.74 | 6.98 | 12.76 | 0.03 | 0.06 | 0.26 | 0.15 | 0.0790 | NM_015980 | HMP19 | HMP19 protein |
| 204147_s_at | 1.83 | 0.1227 | −1.60 | 17.49 | 31.95 | 0.08 | 0.15 | 0.26 | 0.14 | 0.0763 | NM_007111 | TFDP1 | Transcription factor Dp-1 |
| 237606_at | 1.82 | 0.0078 | −2.90 | 4.98 | 9.04 | 0.02 | 0.04 | 0.26 | 0.09 | 0.0056 | AI022073 | CD53 | CD53 molecule |
| 207763_at | 1.81 | 0.0455 | −2.10 | 4.48 | 8.11 | 0.02 | 0.04 | 0.26 | 0.17 | 0.1211 | NM_002962 | S100A5 | S100 calcium binding |
| | | | | | | | | | | | | | protein A5 |
| 1552954_at | 1.81 | 0.1874 | −1.38 | 5.39 | 9.74 | 0.02 | 0.05 | 0.26 | 0.22 | 0.2633 | NM_173668 | C5orf17 | gb: NM_173668.1 |
| | | | | | | | | | | | | | /DB_XREF = gi: 27734704 |
| | | | | | | | | | | | | | /TID = Hs2.363881.1 |
| | | | | | | | | | | | | | /CNT = 3 /FEA = FLmRNA |
| | | | | | | | | | | | | | /TIER = FL /STK = 2 |
| | | | | | | | | | | | | | /LL = 285685 |
| | | | | | | | | | | | | | /UG_GENE = FLJ34836 |
| | | | | | | | | | | | | | /UG = Hs.363881 |
| | | | | | | | | | | | | | /UG_TITLE = hypothetical |
| | | | | | | | | | | | | | protein FLJ34836 |
| | | | | | | | | | | | | | /DEF = Homo sapiens |
| | | | | | | | | | | | | | hypothetical protein |
| | | | | | | | | | | | | | FLJ34836 (FLJ34836), |
| | | | | | | | | | | | | | mRNA. |
| 229328_at | 1.80 | 0.0344 | −2.23 | 5.30 | 9.55 | 0.02 | 0.04 | 0.26 | 0.13 | 0.0513 | T90358 | ZNF540 | /FL = gb: NM_173668.1 |
| | | | | | | | | | | | | | yd43b08.s1 Soares fetal |
| | | | | | | | | | | | | | liver spleen 1NFLS Homo |
| | | | | | | | | | | | | | sapiens cDNA clone |
| | | | | | | | | | | | | | IMAGE: 110967 3′, mRNA |
| | | | | | | | | | | | | | sequence. |
| 1567853_at | 1.80 | 0.0888 | −1.80 | 9.82 | 17.68 | 0.05 | 0.08 | 0.26 | 0.18 | 0.1715 | X52355 | ZNF28 | Zinc finger protein 28 |
| 243918_at | 1.80 | 0.0962 | −1.74 | 8.87 | 15.94 | 0.04 | 0.07 | 0.25 | 0.18 | 0.1371 | AI459554 | LOC647263 | hypothetical protein |
| | | | | | | | | | | | | | LOC647263 |
| 238407_at | 1.79 | 0.0835 | −1.81 | 12.23 | 21.91 | 0.06 | 0.10 | 0.25 | 0.15 | 0.0984 | AI792880 | CAPZA1 | Capping protein (actin |
| | | | | | | | | | | | | | filament) muscle Z-line, |
| | | | | | | | | | | | | | alpha 1 |
| 240803_at | 1.79 | 0.0069 | −2.93 | 5.00 | 8.95 | 0.02 | 0.04 | 0.25 | 0.12 | 0.0400 | AW450626 | C1orf131 | Chromosome 1 open |
| | | | | | | | | | | | | | reading frame 131 |
| 208814_at | 1.79 | 0.7816 | −0.28 | 10.82 | 19.35 | 0.05 | 0.09 | 0.25 | 0.35 | 0.4743 | AA043348 | HSPA4 | Heat shock 70 kDa protein 4 |
| 241624_at | 1.78 | 0.0198 | −2.47 | 14.56 | 25.88 | 0.07 | 0.12 | 0.25 | 0.15 | 0.0813 | BE221330 | LOC389833 | similar to hypothetical |
| | | | | | | | | | | | | | protein MGC27019 |
| 205328_at | 1.78 | 0.1023 | −1.70 | 5.31 | 9.44 | 0.02 | 0.04 | 0.25 | 0.15 | 0.1037 | NM_006984 | CLDN10 | Claudin 10 |
| 207723_s_at | 1.77 | 0.2932 | −1.08 | 30.75 | 54.57 | 0.14 | 0.25 | 0.25 | 0.21 | 0.2559 | NM_002261 | KLRC3 | Killer cell lectin-like |
| | | | | | | | | | | | | | receptor subfamily C, |
| | | | | | | | | | | | | | member 3 |
| 205717_x_at | 1.77 | 0.0019 | −3.47 | 4.85 | 8.61 | 0.02 | 0.04 | 0.25 | 0.10 | 0.0138 | NM_002588 | PCDHGC3 | Protocadherin gamma |
| | | | | | | | | | | | | | subfamily C, 3 |
| 239171_at | 1.77 | 0.1389 | −1.55 | 10.10 | 17.90 | 0.05 | 0.08 | 0.25 | 0.18 | 0.1845 | AA766886 | ADD3 | Adducin 3 (gamma) |
| 241716_at | 1.77 | 0.1694 | −1.42 | 4.46 | 7.88 | 0.02 | 0.04 | 0.25 | 0.19 | 0.2061 | BF965447 | HSPD1 | Heat shock 60 kDa protein |
| | | | | | | | | | | | | | 1 (chaperonin) |
| 221830_at | 1.76 | 0.0936 | −1.78 | 21.26 | 37.48 | 0.10 | 0.17 | 0.25 | 0.13 | 0.0707 | AI302106 | RAP2A | RAP2A, member of RAS |
| | | | | | | | | | | | | | oncogene family |
| 237363_at | 1.76 | 0.1746 | −1.41 | 3.78 | 6.65 | 0.02 | 0.03 | 0.25 | 0.17 | 0.1517 | H15396 | C9orf68 | Chromosome 9 open |
| | | | | | | | | | | | | | reading frame 68 |
| 238635_at | 1.76 | 0.0055 | −3.05 | 6.69 | 11.77 | 0.03 | 0.05 | 0.25 | 0.09 | 0.0099 | W72333 | FLJ21657 | hypothetical protein |
| | | | | | | | | | | | | | FLJ21657 |
| 1560075_at | 1.76 | 0.0321 | −2.28 | 6.65 | 11.68 | 0.03 | 0.05 | 0.24 | 0.14 | 0.0812 | AF075104 | ZNF622 | Zinc finger protein 622 |
| 1566645_at | 1.76 | 0.0058 | −3.11 | 7.79 | 13.68 | 0.04 | 0.06 | 0.24 | 0.11 | 0.0256 | AL050106 | NHEJ1 | Nonhomologous end- |
| | | | | | | | | | | | | | joining factor 1 |
| 1569191_at | 1.75 | 0.1111 | −1.68 | 16.52 | 28.87 | 0.08 | 0.13 | 0.24 | 0.14 | 0.0854 | BC016785 | FLJ44894 | similar to zinc finger |
| | | | | | | | | | | | | | protein 91 |
| 204720_s_at | 1.75 | 0.0273 | −2.42 | 21.95 | 38.31 | 0.10 | 0.18 | 0.24 | 0.10 | 0.0167 | AV729634 | DNAJC6 | DnaJ (Hsp40) homolog, |
| | | | | | | | | | | | | | subfamily C, member 6 |
| 216965_x_at | 1.74 | 0.0065 | −3.03 | 5.71 | 9.96 | 0.03 | 0.05 | 0.24 | 0.12 | 0.0445 | AL139377 | SPG20 | Human DNA sequence |
| | | | | | | | | | | | | | from clone RP11-251J8 on |
| | | | | | | | | | | | | | chromosome 13 Contains 2 |
| | | | | | | | | | | | | | novel genes, the |
| | | | | | | | | | | | | | KIAA0610 gene and a |
| | | | | | | | | | | | | | CpG island, complete |
| | | | | | | | | | | | | | sequence. |
| 217374_x_at | 1.74 | 0.0010 | −3.66 | 14.52 | 25.30 | 0.07 | 0.12 | 0.24 | 0.12 | 0.0374 | AC006033 | STARD3NL | Homo sapiens BAC clone |
| | | | | | | | | | | | | | RP11-121A8 from 7, |
| | | | | | | | | | | | | | complete sequence. |
| 237312_at | 1.74 | 0.0657 | −1.92 | 4.97 | 8.66 | 0.02 | 0.04 | 0.24 | 0.20 | 0.2368 | BF059698 | 237312_at | Transcribed locus |
| 203911_at | 1.74 | 0.1216 | −1.63 | 24.05 | 41.83 | 0.11 | 0.19 | 0.24 | 0.25 | 0.3414 | NM_002885 | RAP1GAP | RAP1 GTPase activating |
| | | | | | | | | | | | | | protein |
| 227952_at | 1.73 | 0.1881 | −1.36 | 119.19 | 206.04 | 0.55 | 0.96 | 0.24 | 0.16 | 0.1412 | AI580142 | LOC402110 | hypothetical LOC402110 |
| 241625_at | 1.73 | 0.0728 | −1.87 | 12.83 | 22.17 | 0.06 | 0.10 | 0.24 | 0.16 | 0.1234 | BE221330 | LOC389833 | similar to hypothetical |
| | | | | | | | | | | | | | protein MGC27019 |
| 217228_s_at | 1.73 | 0.0829 | −1.83 | 4.09 | 7.06 | 0.02 | 0.03 | 0.24 | 0.20 | 0.2409 | AC003079 | ASB4 | Homo sapiens BAC clone |
| | | | | | | | | | | | | | GS1-303P24 from 7, |
| | | | | | | | | | | | | | complete sequence. |
| 229737_at | 1.73 | 0.0480 | −2.07 | 4.63 | 7.99 | 0.02 | 0.04 | 0.24 | 0.13 | 0.0663 | AA526820 | FAM46A | Family with sequence |
| | | | | | | | | | | | | | similarity 46, member A |
| 216298_at | 1.73 | 0.1337 | −1.55 | 26.09 | 45.04 | 0.12 | 0.21 | 0.24 | 0.13 | 0.0685 | AL580863 | TARP | TCR gamma alternate |
| | | | | | | | | | | | | | reading frame protein |
| 1559221_at | 1.73 | 0.3344 | −0.99 | 4.37 | 7.54 | 0.02 | 0.04 | 0.24 | 0.23 | 0.3128 | BC040870 | 1559221_at | Homo sapiens , clone |
| | | | | | | | | | | | | | IMAGE: 5583725, mRNA |
| 220436_at | 1.72 | 0.2165 | −1.29 | 15.19 | 26.17 | 0.07 | 0.12 | 0.24 | 0.22 | 0.2858 | XM_927067 | LOC389722 | similar to cell recognition |
| | | | | | | | | | | | | | molecule CASPR3 |
| 226125_at | 1.72 | 0.0588 | −2.02 | 29.30 | 50.31 | 0.14 | 0.23 | 0.23 | 0.14 | 0.1100 | BF346665 | SLC9A3 | Solute carrier family 9 |
| | | | | | | | | | | | | | (sodium/hydrogen |
| | | | | | | | | | | | | | exchanger), member 3 |
| 230996_at | 1.72 | 0.0059 | −3.03 | 6.46 | 11.09 | 0.03 | 0.05 | 0.23 | 0.12 | 0.0428 | AW024499 | LOC339929 | hypothetical protein |
| | | | | | | | | | | | | | LOC339929 |
| 237756_at | 1.71 | 0.0752 | −1.86 | 5.59 | 9.55 | 0.03 | 0.04 | 0.23 | 0.11 | 0.0384 | AI286028 | KLHL23 | Kelch-like 23 (Drosophila) |
| 234440_at | 1.71 | 0.2546 | −1.18 | 14.43 | 24.61 | 0.07 | 0.11 | 0.23 | 0.17 | 0.1879 | X13954 | 234440_at | Human T-cell receptor |
| | | | | | | | | | | | | | delta-chain (Mann) |
| | | | | | | | | | | | | | V(delta)3 gene. |
| 206290_s_at | 1.70 | 0.0203 | −2.46 | 5.36 | 9.12 | 0.02 | 0.04 | 0.23 | 0.15 | 0.1044 | NM_002924 | RGS7 | Regulator of G-protein |
| | | | | | | | | | | | | | signalling 7 |
| 228802_at | 1.70 | 0.2058 | −1.32 | 43.89 | 74.71 | 0.20 | 0.35 | 0.23 | 0.22 | 0.3052 | BE348466 | RBPMS2 | RNA binding protein with |
| | | | | | | | | | | | | | multiple splicing 2 |
| 241966_at | 1.70 | 0.1706 | −1.41 | 4.27 | 7.24 | 0.02 | 0.03 | 0.23 | 0.14 | 0.0889 | N67810 | MYO5A | Transcribed locus |
| 1557674_s_at | 1.70 | 0.0514 | −2.08 | 6.32 | 10.72 | 0.03 | 0.05 | 0.23 | 0.13 | 0.0847 | AW591765 | EFCAB2 | Transcribed locus |
| 232821_at | 1.69 | 0.2470 | −1.18 | 10.84 | 18.27 | 0.05 | 0.08 | 0.23 | 0.16 | 0.1668 | AI809325 | FAM112A | Family with sequence |
| | | | | | | | | | | | | | similarity 112, member A |
| 238919_at | 1.68 | 0.0368 | −2.25 | 15.85 | 26.68 | 0.07 | 0.12 | 0.23 | 0.12 | 0.0747 | R49295 | PCDH9 | Protocadherin 9 |
| 205361_s_at | 1.68 | 0.1011 | −1.71 | 23.03 | 38.72 | 0.11 | 0.18 | 0.23 | 0.15 | 0.1518 | AI718295 | PFDN4 | Prefoldin subunit 4 |
| 229764_at | 1.68 | 0.0432 | −2.13 | 7.10 | 11.91 | 0.03 | 0.06 | 0.22 | 0.12 | 0.0750 | AW629527 | FAM79B | Family with sequence |
| | | | | | | | | | | | | | similarity 79, member B |
| 223484_at | 1.67 | 0.3408 | −0.98 | 18.72 | 31.34 | 0.09 | 0.15 | 0.22 | 0.24 | 0.3702 | AF228422 | C15orf48 | Chromosome 15 open |
| | | | | | | | | | | | | | reading frame 48 |
| 211049_at | 1.67 | 0.0138 | −2.64 | 6.70 | 11.19 | 0.03 | 0.05 | 0.22 | 0.12 | 0.0672 | BC006356 | TLX2 | T-cell leukemia homeobox 2 |
| 238621_at | 1.67 | 0.0044 | −3.12 | 10.97 | 18.28 | 0.05 | 0.08 | 0.22 | 0.10 | 0.0205 | R67695 | FMN1 | Formin 1 |
| 201131_s_at | 1.66 | 0.1478 | −1.50 | 11.46 | 19.05 | 0.05 | 0.09 | 0.22 | 0.14 | 0.1284 | NM_004360 | CDH1 | Cadherin 1, type 1, E- |
| | | | | | | | | | | | | | cadherin (epithelial) |
| 218948_at | 1.66 | 0.0024 | −3.33 | 20.65 | 34.26 | 0.10 | 0.16 | 0.22 | 0.12 | 0.0491 | AL136679 | QRSL1 | Glutaminyl-tRNA synthase |
| | | | | | | | | | | | | | (glutamine-hydrolyzing)- |
| | | | | | | | | | | | | | like 1 |
| 234864_s_at | 1.65 | 0.0321 | −2.26 | 6.61 | 10.92 | 0.03 | 0.05 | 0.22 | 0.10 | 0.0259 | AK026281 | TRPM6 | Transient receptor |
| | | | | | | | | | | | | | potential cation channel, |
| | | | | | | | | | | | | | subfamily M, member 6 |
| 244856_at | 1.65 | 0.0417 | −2.13 | 4.59 | 7.57 | 0.02 | 0.04 | 0.22 | 0.09 | 0.0182 | AI379784 | FLJ33630 | hypothetical protein |
| | | | | | | | | | | | | | LOC644873 |
| 1556203_a_at | 1.65 | 0.2224 | −1.27 | 12.13 | 19.99 | 0.06 | 0.09 | 0.22 | 0.21 | 0.3095 | AI263819 | LOC653464 | similar to SLIT-ROBO |
| | | | | | | | | | | | | | Rho GTPase-activating |
| | | | | | | | | | | | | | protein 2 (srGAP2) |
| | | | | | | | | | | | | | (Formin-binding protein 2) |
| 220003_at | 1.65 | 0.1735 | −1.40 | 11.11 | 18.31 | 0.05 | 0.09 | 0.22 | 0.12 | 0.0606 | NM_018296 | LRRC36 | Leucine rich repeat |
| | | | | | | | | | | | | | containing 36 |
| 232061_at | 1.65 | 0.1374 | −1.53 | 4.84 | 7.97 | 0.02 | 0.04 | 0.22 | 0.16 | 0.1657 | AA653137 | SDK2 | Sidekick homolog 2 |
| | | | | | | | | | | | | | (chicken) |
| 1567035_at | 1.65 | 0.0037 | −3.18 | 6.04 | 9.94 | 0.03 | 0.05 | 0.22 | 0.11 | 0.0484 | U63828 | C20orf181 | Chromosome 20 open |
| | | | | | | | | | | | | | reading frame 181 |
| 239552_at | 1.64 | 0.4082 | −0.84 | 6.07 | 9.99 | 0.03 | 0.05 | 0.22 | 0.22 | 0.3254 | BF059479 | FLJ14712 | hypothetical protein |
| | | | | | | | | | | | | | FLJ14712 |
| 213683_at | 1.64 | 0.0478 | −2.11 | 7.35 | 12.07 | 0.03 | 0.06 | 0.22 | 0.11 | 0.0512 | AV727634 | ACSL6 | AV727634 HTC Homo |
| | | | | | | | | | | | | | sapiens cDNA clone |
| | | | | | | | | | | | | | HTCAYH08 5′, mRNA |
| | | | | | | | | | | | | | sequence. |
| 220887_at | 1.64 | 0.2535 | −1.17 | 4.79 | 7.85 | 0.02 | 0.04 | 0.21 | 0.21 | 0.2997 | NM_020181 | C14orf162 | Chromosome 14 open |
| | | | | | | | | | | | | | reading frame 162 |
| 1552969_a_at | 1.64 | 0.0155 | −2.60 | 5.54 | 9.07 | 0.03 | 0.04 | 0.21 | 0.09 | 0.0152 | NM_007167 | ZMYM6 | Zinc finger, MYM-type 6 |
| 240949_x_at | 1.64 | 0.2428 | −1.19 | 5.67 | 9.29 | 0.03 | 0.04 | 0.21 | 0.15 | 0.1505 | AI034351 | GALNT10 | UDP-N-acetyl-alpha-D- |
| | | | | | | | | | | | | | galactosamine:polypeptide |
| | | | | | | | | | | | | | N- |
| | | | | | | | | | | | | | acetylgalactosaminyltransferase |
| | | | | | | | | | | | | | 10 (GalNAc-T10) |
| 221111_at | 1.64 | 0.1674 | −1.42 | 4.43 | 7.25 | 0.02 | 0.03 | 0.21 | 0.16 | 0.1855 | NM_018402 | IL26 | Interleukin 26 |
| 233015_at | 1.63 | 0.0090 | −2.81 | 5.10 | 8.34 | 0.02 | 0.04 | 0.21 | 0.11 | 0.0483 | AA732240 | MBNL1 | Muscleblind-like |
| | | | | | | | | | | | | | (Drosophila) |
| 243947_s_at | 1.63 | 0.0100 | −2.77 | 9.02 | 14.74 | 0.04 | 0.07 | 0.21 | 0.11 | 0.0564 | AW300612 | 243947_s_at | Transcribed locus |
| 220703_at | 1.63 | 0.0071 | −2.90 | 19.74 | 32.25 | 0.09 | 0.15 | 0.21 | 0.08 | 0.0068 | NM_018470 | C10orf110 | Chromosome 10 open |
| | | | | | | | | | | | | | reading frame 110 |
| 206619_at | 1.63 | 0.9492 | −0.06 | 4.73 | 7.71 | 0.02 | 0.04 | 0.21 | 0.36 | 0.5664 | NM_014420 | DKK4 | Dickkopf homolog 4 |
| | | | | | | | | | | | | | (Xenopus laevis) |
| 1564707_x_at | 1.63 | 0.4035 | −0.85 | 4.19 | 6.83 | 0.02 | 0.03 | 0.21 | 0.23 | 0.3675 | AF110329 | GLS2 | Homo sapiens |
| | | | | | | | | | | | | | mitochondrial glutaminase |
| | | | | | | | | | | | | | pseudogene, nuclear |
| | | | | | | | | | | | | | pseudogene, complete |
| | | | | | | | | | | | | | sequence. |
| 237077_at | 1.63 | 0.9369 | −0.08 | 4.54 | 7.40 | 0.02 | 0.03 | 0.21 | 0.31 | 0.5008 | AI821895 | ACPP | Acid phosphatase, prostate |
| 240997_at | 1.63 | 0.1330 | −1.56 | 4.69 | 7.64 | 0.02 | 0.04 | 0.21 | 0.13 | 0.1070 | AA455864 | LOC131873 | hypothetical protein |
| | | | | | | | | | | | | | LOC131873 |
| 224937_at | 1.63 | 0.5773 | −0.56 | 9.38 | 15.29 | 0.04 | 0.07 | 0.21 | 0.17 | 0.2123 | BF311866 | PTGFRN | 601897391F1 |
| | | | | | | | | | | | | | NIH_MGC_19 Homo |
| | | | | | | | | | | | | | sapiens cDNA clone |
| | | | | | | | | | | | | | IMAGE: 4126486 5′, |
| | | | | | | | | | | | | | mRNA sequence. |
| 229313_at | 1.63 | 0.0436 | −2.11 | 10.95 | 17.83 | 0.05 | 0.08 | 0.21 | 0.10 | 0.0268 | AA843962 | TMEM16E | Transmembrane protein |
| | | | | | | | | | | | | | 16E |
| 1561016_at | 1.63 | 0.0703 | −1.89 | 4.03 | 6.55 | 0.02 | 0.03 | 0.21 | 0.14 | 0.1428 | AF086084 | COMMD10 | COMM domain containing |
| | | | | | | | | | | | | | 10 |
| 238151_at | 1.63 | 0.3178 | −1.03 | 22.36 | 36.37 | 0.10 | 0.17 | 0.21 | 0.19 | 0.2786 | BF511636 | TUBB6 | Tubulin, beta 6 |
| 240607_at | 1.62 | 0.0202 | −2.51 | 19.33 | 31.32 | 0.09 | 0.15 | 0.21 | 0.10 | 0.0351 | AI692560 | C22orf35 | Hypothetical protein |
| | | | | | | | | | | | | | LOC150271 |
| 227835_at | 1.62 | 0.6449 | −0.47 | 18.97 | 30.72 | 0.09 | 0.14 | 0.21 | 0.18 | 0.2478 | T86830 | LOC389831 | hypothetical gene |
| | | | | | | | | | | | | | supported by AL713796 |
| 235953_at | 1.62 | 0.0101 | −2.85 | 6.78 | 10.97 | 0.03 | 0.05 | 0.21 | 0.09 | 0.0300 | AA776810 | ZNF610 | Zinc finger protein 610 |
| 202733_at | 1.62 | 0.0027 | −3.30 | 5.12 | 8.27 | 0.02 | 0.04 | 0.21 | 0.13 | 0.0901 | NM_004199 | P4HA2 | Procollagen-proline, 2- |
| | | | | | | | | | | | | | oxoglutarate 4- |
| | | | | | | | | | | | | | dioxygenase (proline 4- |
| | | | | | | | | | | | | | hydroxylase), alpha |
| | | | | | | | | | | | | | polypeptide II |
| 201579_at | 1.61 | 0.0439 | −2.19 | 8.87 | 14.32 | 0.04 | 0.07 | 0.21 | 0.15 | 0.1662 | NM_005245 | FAT | FAT tumor suppressor |
| | | | | | | | | | | | | | homolog 1 (Drosophila) |
| 238582_at | 1.61 | 0.0162 | −2.56 | 4.12 | 6.64 | 0.02 | 0.03 | 0.21 | 0.10 | 0.0313 | AI290561 | C21orf2 | Transcribed locus |
| 234896_at | 1.61 | 0.0053 | −3.02 | 5.02 | 8.09 | 0.02 | 0.04 | 0.21 | 0.09 | 0.0252 | AJ012680 | C1orf5 | Homo sapiens gene |
| | | | | | | | | | | | | | encoding hypothetical |
| | | | | | | | | | | | | | protein with HTH motif. |
| 205289_at | 1.61 | 0.7243 | −0.36 | 18.68 | 30.12 | 0.09 | 0.14 | 0.21 | 0.23 | 0.3694 | AA583044 | BMP2 | Bone morphogenetic |
| | | | | | | | | | | | | | protein 2 |
| 239623_at | 1.61 | 0.0242 | −2.40 | 6.83 | 11.01 | 0.03 | 0.05 | 0.21 | 0.11 | 0.0678 | N93197 | FLJ44606 | hypothetical gene |
| | | | | | | | | | | | | | supported by AK126569 |
| 238311_at | 1.61 | 0.1726 | −1.42 | 6.26 | 10.07 | 0.03 | 0.05 | 0.21 | 0.17 | 0.2423 | BF940192 | KIAA0776 | KIAA0776 |
| 203476_at | 1.61 | 0.0542 | −2.06 | 23.97 | 38.55 | 0.11 | 0.18 | 0.21 | 0.15 | 0.1641 | NM_006670 | TPBG | Trophoblast glycoprotein |
| 205336_at | 1.61 | 0.1906 | −1.34 | 5.54 | 8.91 | 0.03 | 0.04 | 0.21 | 0.16 | 0.2030 | NM_002854 | PVALB | Parvalbumin |
| 206582_s_at | 1.61 | 0.4475 | −0.78 | 5.88 | 9.45 | 0.03 | 0.04 | 0.21 | 0.22 | 0.3615 | NM_005682 | GPR56 | G protein-coupled receptor |
| | | | | | | | | | | | | | 56 |
| 221016_s_at | 1.61 | 0.2654 | −1.14 | 4.81 | 7.73 | 0.02 | 0.04 | 0.21 | 0.14 | 0.1325 | NM_031283 | TCF7L1 | Transcription factor 7-like |
| | | | | | | | | | | | | | 1 (T-cell specific, HMG- |
| | | | | | | | | | | | | | box) |
| 242146_at | 1.60 | 0.0049 | −3.09 | 62.32 | 99.88 | 0.29 | 0.46 | 0.20 | 0.07 | 0.0056 | AA872471 | SNRPA1 | Small nuclear |
| | | | | | | | | | | | | | ribonucleoprotein |
| | | | | | | | | | | | | | polypeptide A′ |
| 229623_at | 1.60 | 0.0175 | −2.60 | 5.91 | 9.45 | 0.03 | 0.04 | 0.20 | 0.10 | 0.0544 | BF508344 | FLJ12993 | hypothetical LOC441027 |
| 224822_at | 1.60 | 0.2540 | −1.17 | 21.83 | 34.91 | 0.10 | 0.16 | 0.20 | 0.18 | 0.2568 | AA524250 | DLC1 | Deleted in liver cancer 1 |
| 225952_at | 1.60 | 0.4049 | −0.85 | 4.22 | 6.75 | 0.02 | 0.03 | 0.20 | 0.20 | 0.2913 | BF338560 | FLYWCH1 | FLYWCH-type zinc finger 1 |
| 1566102_at | 1.60 | 0.0960 | −1.73 | 5.73 | 9.16 | 0.03 | 0.04 | 0.20 | 0.11 | 0.0745 | AL137329 | TTLL5 | Homo sapiens genomic |
| | | | | | | | | | | | | | DNA; cDNA |
| | | | | | | | | | | | | | DKFZp434E2172 (from |
| | | | | | | | | | | | | | clone DKFZp434E2172). |
| 215101_s_at | 1.59 | 0.1059 | −1.67 | 11.92 | 19.01 | 0.05 | 0.09 | 0.20 | 0.12 | 0.1040 | BG166705 | CXCL5 | Chemokine (C-X-C motif) |
| | | | | | | | | | | | | | ligand 5 |
| 226498_at | 1.59 | 0.0085 | −2.86 | 8.44 | 13.46 | 0.04 | 0.06 | 0.20 | 0.09 | 0.0213 | AA149648 | FLT1 | Fms-related tyrosine |
| | | | | | | | | | | | | | kinase 1 (vascular |
| | | | | | | | | | | | | | endothelial growth |
| | | | | | | | | | | | | | factor/vascular |
| | | | | | | | | | | | | | permeability factor |
| | | | | | | | | | | | | | receptor) |
| 1552373_s_at | 1.59 | 0.3081 | −1.04 | 4.98 | 7.92 | 0.02 | 0.04 | 0.20 | 0.13 | 0.1373 | BC016358 | LOC132321 | hypothetical protein |
| | | | | | | | | | | | | | LOC132321 |
| 240287_at | 1.59 | 0.0415 | −2.14 | 4.22 | 6.70 | 0.02 | 0.03 | 0.20 | 0.10 | 0.0480 | BG236136 | LOC730249 | Transcribed locus, strongly |
| | | | | | | | | | | | | | similar to XP_292184.4 |
| | | | | | | | | | | | | | PREDICTED: similar to |
| | | | | | | | | | | | | | immune-responsive gene 1 |
| | | | | | | | | | | | | | [Homo sapiens] |
| 205741_s_at | 1.59 | 0.0606 | −1.97 | 4.40 | 6.97 | 0.02 | 0.03 | 0.20 | 0.13 | 0.1185 | NM_001392 | DTNA | Dystrobrevin, alpha |
| 238567_at | 1.58 | 0.1326 | −1.56 | 5.27 | 8.34 | 0.02 | 0.04 | 0.20 | 0.20 | 0.3164 | AW779536 | SGPP2 | Sphingosine-1-phosphate |
| | | | | | | | | | | | | | phosphotase 2 |
| 222055_at | 1.58 | 0.0961 | −1.73 | 8.13 | 12.86 | 0.04 | 0.06 | 0.20 | 0.11 | 0.0687 | AA723370 | FAHD2A | Fumarylacetoacetate |
| | | | | | | | | | | | | | hydrolase domain |
| | | | | | | | | | | | | | containing 2A |
| 1563750_at | 1.58 | 0.0112 | −2.78 | 7.20 | 11.40 | 0.03 | 0.05 | 0.20 | 0.08 | 0.0137 | AL833376 | FLJ43944 | FLJ43944 protein |
| 1569337_at | 1.58 | 0.1186 | −1.62 | 5.07 | 8.02 | 0.02 | 0.04 | 0.20 | 0.16 | 0.2227 | BC032417 | SLC5A9 | Solute carrier family 5 |
| | | | | | | | | | | | | | (sodium/glucose |
| | | | | | | | | | | | | | cotransporter), member 9 |
| 243605_at | 1.58 | 0.2646 | −1.15 | 25.11 | 39.68 | 0.12 | 0.18 | 0.20 | 0.19 | 0.3016 | AW627671 | CPEB2 | Cytoplasmic |
| | | | | | | | | | | | | | polyadenylation element |
| | | | | | | | | | | | | | binding protein 2 |
| 206413_s_at | 1.58 | 0.7167 | −0.37 | 6.11 | 9.65 | 0.03 | 0.04 | 0.20 | 0.26 | 0.4615 | NM_004918 | TCL1B | T-cell leukemia/lymphoma |
| | | | | | | | | | | | | | 1B |
| 242056_at | 1.58 | 0.0379 | −2.19 | 5.19 | 8.20 | 0.02 | 0.04 | 0.20 | 0.12 | 0.0845 | AI793200 | TRIM45 | Tripartite motif-containing |
| | | | | | | | | | | | | | 45 |
| 1556879_at | 1.58 | 0.0283 | −2.34 | 4.18 | 6.59 | 0.02 | 0.03 | 0.20 | 0.11 | 0.0656 | AW339812 | na | CDNA FLJ39461 fis, |
| | | | | | | | | | | | | | clone PROST2011660 |
| 244625_at | 1.58 | 0.0331 | −2.32 | 19.41 | 30.61 | 0.09 | 0.14 | 0.20 | 0.08 | 0.0217 | AW629478 | RERE | Arginine-glutamic acid |
| | | | | | | | | | | | | | dipeptide (RE) repeats |
| 216823_at | 1.57 | 0.0344 | −2.23 | 37.26 | 58.58 | 0.17 | 0.27 | 0.20 | 0.10 | 0.0580 | AL356115 | KIAA1128 | Human DNA sequence |
| | | | | | | | | | | | | | from clone RP11-486O22 |
| | | | | | | | | | | | | | on chromosome 10 |
| | | | | | | | | | | | | | Contains the 3′ end of a |
| | | | | | | | | | | | | | novel gene (KIAA1128, |
| | | | | | | | | | | | | | FLJ14262, FLJ25809), the |
| | | | | | | | | | | | | | 5′ end of a Siah-interacting |
| | | | | | | | | | | | | | protein (SIP) (calcyclin |
| | | | | | | | | | | | | | binding protein) |
| | | | | | | | | | | | | | pseudogene, ribosomal |
| | | | | | | | | | | | | | protein S3A pseudogene 5 |
| | | | | | | | | | | | | | (RPS3AP5) and two CpG |
| | | | | | | | | | | | | | islands, complete |
| | | | | | | | | | | | | | sequence. |
| 229797_at | 1.57 | 0.0627 | −1.94 | 10.73 | 16.86 | 0.05 | 0.08 | 0.20 | 0.11 | 0.0740 | AI636080 | MCOLN3 | Mucolipin 3 |
| 202062_s_at | 1.57 | 0.1832 | −1.39 | 28.10 | 44.14 | 0.13 | 0.20 | 0.20 | 0.18 | 0.2794 | NM_005065 | SEL1L | Sel-1 suppressor of lin-12- |
| | | | | | | | | | | | | | like (C. elegans) |
| 234384_at | 1.57 | 0.2755 | −1.12 | 7.58 | 11.90 | 0.03 | 0.06 | 0.20 | 0.11 | 0.0887 | Y13187 | DMD | Homo sapiens partial |
| | | | | | | | | | | | | | DMD gene, intron 11, |
| | | | | | | | | | | | | | isolate 381. |
| 237771_s_at | 1.57 | 0.2068 | −1.30 | 5.29 | 8.31 | 0.02 | 0.04 | 0.20 | 0.17 | 0.2607 | AW340015 | 237771_s_at | Transcribed locus |
| 210546_x_at | 1.57 | 0.4343 | −0.79 | 7.66 | 12.03 | 0.04 | 0.06 | 0.20 | 0.25 | 0.4506 | U87459 | CTAG1B | Cancer/testis antigen 1B |
| 213201_s_at | 1.57 | 0.2617 | −1.15 | 5.69 | 8.92 | 0.03 | 0.04 | 0.20 | 0.26 | 0.4496 | AJ011712 | TNNT1 | Homo sapiens TNNT1 |
| | | | | | | | | | | | | | gene, exons 1-11 (and |
| | | | | | | | | | | | | | joined CDS). |
| 225721_at | 1.57 | 0.0264 | −2.35 | 4.41 | 6.92 | 0.02 | 0.03 | 0.20 | 0.11 | 0.0769 | AI658662 | SYNPO2 | Synaptopodin 2 |
| 1557432_at | 1.57 | 0.1396 | −1.52 | 4.76 | 7.46 | 0.02 | 0.03 | 0.19 | 0.14 | 0.1536 | BQ003426 | RASAL2 | RAS protein activator like 2 |
| 237737_at | 1.57 | 0.0230 | −2.44 | 8.58 | 13.44 | 0.04 | 0.06 | 0.19 | 0.09 | 0.0361 | AI359676 | SH3GL3 | Transcribed locus |
| 205942_s_at | 1.57 | 0.0684 | −1.90 | 8.46 | 13.25 | 0.04 | 0.06 | 0.19 | 0.12 | 0.0946 | NM_005622 | ACSM3 | Acyl-CoA synthetase |
| | | | | | | | | | | | | | medium-chain family |
| | | | | | | | | | | | | | member 3 |
| 236239_at | 1.57 | 0.0130 | −2.66 | 7.19 | 11.25 | 0.03 | 0.05 | 0.19 | 0.12 | 0.0928 | AW609310 | XPNPEP1 | X-prolyl aminopeptidase |
| | | | | | | | | | | | | | (aminopeptidase P) 1, |
| | | | | | | | | | | | | | soluble |
| 1567237_at | 1.57 | 0.0259 | −2.37 | 4.24 | 6.64 | 0.02 | 0.03 | 0.19 | 0.13 | 0.1438 | X64978 | OR2L2 | Olfactory receptor, family |
| | | | | | | | | | | | | | 2, subfamily L, member 2 |
| 1566144_at | 1.56 | 0.2113 | −1.28 | 5.21 | 8.15 | 0.02 | 0.04 | 0.19 | 0.18 | 0.2620 | AK098337 | SH3GL3 | CDNA FLJ41018 fis, |
| | | | | | | | | | | | | | clone UTERU2018881 |
| 244638_at | 1.56 | 0.0066 | −2.95 | 9.72 | 15.21 | 0.04 | 0.07 | 0.19 | 0.07 | 0.0040 | AW954477 | SUCLG1 | Succinate-CoA ligase, |
| | | | | | | | | | | | | | GDP-forming, alpha |
| | | | | | | | | | | | | | subunit |
| 242897_at | 1.56 | 0.1007 | −1.71 | 6.76 | 10.57 | 0.03 | 0.05 | 0.19 | 0.16 | 0.2197 | AA641796 | 242897_at | ns19b01.s1 |
| | | | | | | | | | | | | | NCI_CGAP_GCB1 Homo |
| | | | | | | | | | | | | | sapiens cDNA clone |
| | | | | | | | | | | | | | IMAGE: 1184041 3′, |
| | | | | | | | | | | | | | mRNA sequence. |
| 243591_at | 1.56 | 0.0970 | −1.72 | 6.95 | 10.87 | 0.03 | 0.05 | 0.19 | 0.14 | 0.1580 | AI887749 | LAMC1 | Laminin, gamma 1 |
| | | | | | | | | | | | | | (formerly LAMB2) |
| 214075_at | 1.56 | 0.0248 | −2.38 | 14.92 | 23.33 | 0.07 | 0.11 | 0.19 | 0.14 | 0.1639 | AI984136 | NENF | Neuron derived |
| | | | | | | | | | | | | | neurotrophic factor |
| 207808_s_at | 1.56 | 0.3222 | −1.01 | 79.49 | 124.24 | 0.37 | 0.58 | 0.19 | 0.15 | 0.2060 | NM_000313 | PROS1 | Protein S (alpha) |
| 1557094_at | 1.56 | 0.5510 | −0.61 | 9.15 | 14.30 | 0.04 | 0.07 | 0.19 | 0.20 | 0.3413 | BC029890 | ANXA8 | Homo sapiens annexin A8, |
| | | | | | | | | | | | | | mRNA (cDNA clone |
| | | | | | | | | | | | | | IMAGE: 5175354), |
| | | | | | | | | | | | | | containing frame-shift |
| | | | | | | | | | | | | | errors. |
| 1560373_a_at | 1.56 | 0.0270 | −2.37 | 17.97 | 28.04 | 0.08 | 0.13 | 0.19 | 0.08 | 0.0209 | AI800806 | 1560373_a_at | CDNA FLJ34680 fis, |
| | | | | | | | | | | | | | clone LIVER2003524 |
| 201249_at | 1.56 | 0.4798 | −0.72 | 10.18 | 15.88 | 0.05 | 0.07 | 0.19 | 0.21 | 0.3766 | AI091047 | SLC2A1 | Solute carrier family 2 |
| | | | | | | | | | | | | | (facilitated glucose |
| | | | | | | | | | | | | | transporter), member 1 |
| 234064_at | 1.56 | 0.0546 | −2.02 | 7.14 | 11.12 | 0.03 | 0.05 | 0.19 | 0.09 | 0.0390 | AK024900 | AP2B1 | CDNA: FLJ21247 fis, |
| | | | | | | | | | | | | | clone COL01205 |
| 241866_at | 1.56 | 0.0125 | −2.67 | 23.95 | 37.31 | 0.11 | 0.17 | 0.19 | 0.07 | 0.0034 | AW975728 | SLC16A7 | Solute carrier family 16 |
| | | | | | | | | | | | | | (monocarboxylic acid |
| | | | | | | | | | | | | | transporters), member 7 |
| 242174_at | 1.56 | 0.0009 | −3.71 | 5.00 | 7.79 | 0.02 | 0.04 | 0.19 | 0.07 | 0.0035 | AI732542 | ZBTB10 | ni36g03.x5 |
| | | | | | | | | | | | | | NCI_CGAP_Lu1 Homo |
| | | | | | | | | | | | | | sapiens cDNA clone |
| | | | | | | | | | | | | | IMAGE: 978964 3′ similar |
| | | | | | | | | | | | | | to contains Alu repetitive |
| | | | | | | | | | | | | | element; contains element |
| | | | | | | | | | | | | | TAR1 TAR1 repetitive |
| | | | | | | | | | | | | | element; mRNA |
| | | | | | | | | | | | | | sequence. |
| 1554234_at | 1.56 | 0.0166 | −2.60 | 7.99 | 12.44 | 0.04 | 0.06 | 0.19 | 0.08 | 0.0224 | BC034999 | KATNAL2 | Katanin p60 subunit A-like 2 |
| 1552658_a_at | 1.55 | 0.0757 | −1.85 | 6.19 | 9.63 | 0.03 | 0.04 | 0.19 | 0.11 | 0.0740 | NM_014903 | NAV3 | Neuron navigator 3 |
| 227701_at | 1.55 | 0.0016 | −3.49 | 167.67 | 260.66 | 0.77 | 1.21 | 0.19 | 0.06 | 0.0020 | AK024739 | C10orf118 | Chromosome 10 open |
| | | | | | | | | | | | | | reading frame 118 |
| 1564056_at | 1.55 | 0.2915 | −1.08 | 5.98 | 9.30 | 0.03 | 0.04 | 0.19 | 0.13 | 0.1370 | AL832887 | 1564056_at | MRNA; cDNA |
| | | | | | | | | | | | | | DKFZp667N093 (from |
| | | | | | | | | | | | | | clone DKFZp667N093) |
| 1554408_a_at | 1.55 | 0.1259 | 1.58 | 6.99 | 10.84 | 0.03 | 0.05 | 0.19 | 0.08 | 0.0129 | BC007986 | TK1 | Thymidine kinase 1, |
| | | | | | | | | | | | | | soluble |
| 207854_at | 1.55 | 0.4386 | −0.79 | 70.08 | 108.74 | 0.32 | 0.50 | 0.19 | 0.18 | 0.2923 | NM_002102 | GYPE | Glycophorin B (MNS |
| | | | | | | | | | | | | | blood group) |
| 243412_at | 1.55 | 0.1300 | −1.58 | 4.29 | 6.65 | 0.02 | 0.03 | 0.19 | 0.11 | 0.0942 | AW182342 | EMR4 | Egf-like module |
| | | | | | | | | | | | | | containing, mucin-like, |
| | | | | | | | | | | | | | hormone receptor-like 4 |
| 1556202_at | 1.55 | 0.0493 | −2.11 | 9.49 | 14.70 | 0.04 | 0.07 | 0.19 | 0.11 | 0.0945 | AI263819 | LOC653464 | similar to SLIT-ROBO |
| | | | | | | | | | | | | | Rho GTPase-activating |
| | | | | | | | | | | | | | protein 2 (srGAP2) |
| | | | | | | | | | | | | | (Formin-binding protein 2) |
| 215819_s_at | 1.55 | 0.6020 | −0.53 | 36.06 | 55.74 | 0.17 | 0.26 | 0.19 | 0.24 | 0.4311 | N53959 | RHCE | Rh blood group, CcEe |
| | | | | | | | | | | | | | antigens |
| 235644_at | 1.55 | 0.0864 | −1.79 | 9.35 | 14.45 | 0.04 | 0.07 | 0.19 | 0.10 | 0.0721 | BF213953 | FLJ32745 | hypothetical protein |
| | | | | | | | | | | | | | FLJ32745 |
| 1553994_at | 1.55 | 0.3599 | −0.94 | 10.52 | 16.26 | 0.05 | 0.08 | 0.19 | 0.16 | 0.2471 | BC015940 | NT5E | 5′-nucleotidase, ecto |
| | | | | | | | | | | | | | (CD73) |
| 1559459_at | 1.55 | 0.2100 | −1.29 | 8.62 | 13.32 | 0.04 | 0.06 | 0.19 | 0.13 | 0.1559 | BC043571 | LOC613266 | hypothetical LOC613266 |
| 1561708_at | 1.54 | 0.0264 | −2.35 | 4.48 | 6.92 | 0.02 | 0.03 | 0.19 | 0.10 | 0.0676 | BC012928 | C6orf150 | Chromosome 6 open |
| | | | | | | | | | | | | | reading frame 150 |
| 231226_at | 1.54 | 0.3400 | −0.97 | 5.31 | 8.20 | 0.02 | 0.04 | 0.19 | 0.13 | 0.1294 | BF196752 | LOC728142 | CDNA clone |
| | | | | | | | | | | | | | IMAGE: 5300185 |
| 1552425_a_at | 1.54 | 0.2394 | −1.21 | 8.28 | 12.78 | 0.04 | 0.06 | 0.19 | 0.17 | 0.2635 | NM_152467 | KLHL10 | Kelch-like 10 (Drosophila) |
| 39549_at | 1.54 | 0.0810 | −1.82 | 6.45 | 9.96 | 0.03 | 0.05 | 0.19 | 0.10 | 0.0677 | AI743090 | NPAS2 | Neuronal PAS domain |
| | | | | | | | | | | | | | protein 2 |
| 1555465_at | 1.54 | 0.0369 | −2.19 | 4.40 | 6.77 | 0.02 | 0.03 | 0.19 | 0.13 | 0.1374 | AY083533 | MCOLN2 | Mucolipin 2 |
| 230703_at | 1.54 | 0.1505 | −1.49 | 9.40 | 14.49 | 0.04 | 0.07 | 0.19 | 0.15 | 0.2154 | AA001543 | C14orf32 | Chromosome 14 open |
| | | | | | | | | | | | | | reading frame 32 |
| 229125_at | 1.54 | 0.1121 | −1.64 | 4.46 | 6.88 | 0.02 | 0.03 | 0.19 | 0.11 | 0.0845 | AA456955 | ANKRD38 | Ankyrin repeat domain 38 |
| 238164_at | 1.54 | 0.1906 | −1.36 | 17.40 | 26.80 | 0.08 | 0.12 | 0.19 | 0.15 | 0.2324 | AA741061 | USP6NL | USP6 N-terminal like |
| 1553499_s_at | 1.54 | 0.3335 | −0.98 | 4.88 | 7.52 | 0.02 | 0.03 | 0.19 | 0.14 | 0.1662 | AY185496 | SERPINA9 | Serpin peptidase inhibitor, |
| | | | | | | | | | | | | | clade A (alpha-1 |
| | | | | | | | | | | | | | antiproteinase, antitrypsin), |
| | | | | | | | | | | | | | member 9 |
| 211190_x_at | 1.54 | 0.0818 | −1.84 | 5.49 | 8.45 | 0.03 | 0.04 | 0.19 | 0.12 | 0.1261 | AF054817 | CD84 | CD84 molecule |
| 231600_at | 1.54 | 0.0789 | −1.83 | 132.64 | 203.98 | 0.61 | 0.95 | 0.19 | 0.13 | 0.1443 | AI657064 | CLEC12B | C-type lectin domain |
| | | | | | | | | | | | | | family 12 member B |
| 202411_at | 1.54 | 0.7852 | −0.28 | 52.02 | 79.99 | 0.24 | 0.37 | 0.19 | 0.22 | 0.4000 | NM_005532 | IFI27 | Interferon, alpha-inducible |
| | | | | | | | | | | | | | protein 27 |
| 223463_at | 1.54 | 0.0444 | −2.13 | 19.42 | 29.86 | 0.09 | 0.14 | 0.19 | 0.07 | 0.0132 | AF161486 | RAB23 | RAB23, member RAS |
| | | | | | | | | | | | | | oncogene family |
| 1559491_at | 1.54 | 0.0048 | −3.10 | 6.59 | 10.13 | 0.03 | 0.05 | 0.19 | 0.11 | 0.0682 | AL390180 | TNRC17 | MRNA; cDNA |
| | | | | | | | | | | | | | DKFZp761L149 (from |
| | | | | | | | | | | | | | clone DKFZp761L149) |
| 1556796_at | 1.54 | 0.1128 | −1.64 | 4.33 | 6.65 | 0.02 | 0.03 | 0.19 | 0.12 | 0.1043 | AW187990 | LOC401061 | CDNA FLJ32776 fis, |
| | | | | | | | | | | | | | clone TESTI2002048 |
| 241696_at | 1.54 | 0.0298 | −2.29 | 5.77 | 8.86 | 0.03 | 0.04 | 0.19 | 0.11 | 0.0796 | AA280904 | C9orf39 | Chromosome 9 open |
| | | | | | | | | | | | | | reading frame 39 |
| 205399_at | 1.54 | 0.4672 | −0.74 | 4.55 | 6.98 | 0.02 | 0.03 | 0.19 | 0.15 | 0.2143 | NM_004734 | DCAMKL1 | Doublecortin and CaM |
| | | | | | | | | | | | | | kinase-like 1 |
| 222727_s_at | 1.54 | 0.0062 | −2.96 | 5.04 | 7.74 | 0.02 | 0.04 | 0.19 | 0.07 | 0.0121 | AI339568 | SLC24A6 | Solute carrier family 24 |
| | | | | | | | | | | | | | (sodium/potassium/calcium |
| | | | | | | | | | | | | | exchanger), member 6 |
| 1564932_at | 1.53 | 0.0733 | −1.88 | 14.64 | 22.47 | 0.07 | 0.10 | 0.19 | 0.12 | 0.1321 | AL049311 | LYST | MRNA; cDNA |
| | | | | | | | | | | | | | DKFZp564B226 (from |
| | | | | | | | | | | | | | clone DKFZp564B226) |
| 221805_at | 1.53 | 0.0409 | −2.14 | 14.97 | 22.96 | 0.07 | 0.11 | 0.19 | 0.10 | 0.0553 | AL537457 | NEFL | Neurofilament, light |
| | | | | | | | | | | | | | polypeptide 68 kDa |
| 239964_at | 1.53 | 0.4667 | −0.74 | 12.29 | 18.85 | 0.06 | 0.09 | 0.19 | 0.21 | 0.3911 | AA207142 | TCL6 | T-cell leukemia/lymphoma 6 |
| 234837_at | 1.53 | 0.1902 | −1.35 | 4.25 | 6.51 | 0.02 | 0.03 | 0.19 | 0.15 | 0.2224 | AL049349 | MSRA | Methionine sulfoxide |
| | | | | | | | | | | | | | reductase A |
| 211820_x_at | 1.53 | 0.5649 | −0.59 | 80.81 | 123.86 | 0.37 | 0.58 | 0.19 | 0.19 | 0.3272 | U00179 | GYPA | Glycophorin A (MNS |
| | | | | | | | | | | | | | blood group) |
| 234785_at | 1.53 | 0.0452 | −2.10 | 6.73 | 10.31 | 0.03 | 0.05 | 0.19 | 0.12 | 0.1352 | AK025047 | FLJ21394 | Homo sapiens cDNA: |
| | | | | | | | | | | | | | FLJ21394 fis, clone |
| | | | | | | | | | | | | | COL03536. |
| 1569608_x_at | 1.53 | 0.1997 | −1.31 | 6.50 | 9.96 | 0.03 | 0.05 | 0.19 | 0.20 | 0.3592 | BC016022 | LOC643187 | Homo sapiens, clone |
| | | | | | | | | | | | | | IMAGE: 4720764, mRNA |
| 231983_at | 1.53 | 0.2013 | −1.33 | 22.41 | 34.31 | 0.10 | 0.16 | 0.19 | 0.17 | 0.2816 | BG471870 | C1orf69 | 602513345F1 |
| | | | | | | | | | | | | | NIH_MGC_16 Homo |
| | | | | | | | | | | | | | sapiens cDNA clone |
| | | | | | | | | | | | | | IMAGE: 4636114 5′, |
| | | | | | | | | | | | | | mRNA sequence. |
| 208200_at | 1.53 | 0.3951 | −0.87 | 23.05 | 35.28 | 0.11 | 0.16 | 0.18 | 0.22 | 0.4086 | NM_000575 | IL1A | Interleukin 1, alpha |
| 230951_at | 1.53 | 0.1802 | −1.38 | 8.65 | 13.23 | 0.04 | 0.06 | 0.18 | 0.12 | 0.1392 | AW242920 | EPB41L5 | Erythrocyte membrane |
| | | | | | | | | | | | | | protein band 4.1 like 5 |
| 244280_at | 1.53 | 0.1966 | −1.33 | 14.28 | 21.80 | 0.07 | 0.10 | 0.18 | 0.15 | 0.2261 | W46364 | MAGED4 | Homo sapiens, clone |
| | | | | | | | | | | | | | IMAGE: 5583725, mRNA |
| 240114_s_at | 1.53 | 0.0003 | −4.14 | 5.24 | 7.99 | 0.02 | 0.04 | 0.18 | 0.07 | 0.0065 | AI927971 | MGC13034 | hypothetical protein |
| | | | | | | | | | | | | | MGC13034 |
| 214734_at | 1.53 | 0.0373 | −2.19 | 10.18 | 15.52 | 0.05 | 0.07 | 0.18 | 0.11 | 0.0867 | AB014524 | EXPH5 | Exophilin 5 |
| 215973_at | 1.52 | 0.0217 | −2.43 | 8.22 | 12.53 | 0.04 | 0.06 | 0.18 | 0.10 | 0.0610 | AF036973 | C6orf12 | Chromosome 6 open |
| | | | | | | | | | | | | | reading frame 12 |
| 239724_at | 1.52 | 0.1968 | −1.33 | 7.48 | 11.40 | 0.03 | 0.05 | 0.18 | 0.15 | 0.2117 | AI653368 | WDR26 | WD repeat domain 26 |
| 225283_at | 1.52 | 0.0237 | −2.40 | 175.12 | 266.82 | 0.81 | 1.24 | 0.18 | 0.08 | 0.0145 | AV701177 | ARRDC4 | Arrestin domain |
| | | | | | | | | | | | | | containing 4 |
| 220264_s_at | 1.52 | 0.0208 | −2.47 | 6.98 | 10.63 | 0.03 | 0.05 | 0.18 | 0.12 | 0.1432 | NM_020960 | GPR107 | G protein-coupled receptor |
| | | | | | | | | | | | | | 107 |
| 208451_s_at | 1.52 | 0.5405 | −0.62 | 29.78 | 45.34 | 0.14 | 0.21 | 0.18 | 0.26 | 0.4798 | NM_000592 | C4B | Homo sapiens complement |
| | | | | | | | | | | | | | component 4B (Childo |
| | | | | | | | | | | | | | blood group) (C4B), |
| | | | | | | | | | | | | | mRNA. |
| 243106_at | 1.52 | 0.0684 | −1.90 | 153.64 | 233.93 | 0.71 | 1.09 | 0.18 | 0.12 | 0.1322 | AA916861 | CLEC12A | C-type lectin domain |
| | | | | | | | | | | | | | family 12, member A |
| 226497_s_at | 1.52 | 0.0883 | −1.79 | 17.62 | 26.80 | 0.08 | 0.12 | 0.18 | 0.10 | 0.0664 | AA149648 | FLT1 | Fms-related tyrosine |
| | | | | | | | | | | | | | kinase 1 (vascular |
| | | | | | | | | | | | | | endothelial growth |
| | | | | | | | | | | | | | factor/vascular |
| | | | | | | | | | | | | | permeability factor |
| | | | | | | | | | | | | | receptor) |
| 210029_at | 1.52 | 0.2808 | −1.10 | 27.48 | 41.79 | 0.13 | 0.19 | 0.18 | 0.15 | 0.2389 | M34455 | INDO | Indoleamine-pyrrole 2,3 |
| | | | | | | | | | | | | | dioxygenase |
| 241394_at | 1.52 | 0.1380 | −1.53 | 6.31 | 9.58 | 0.03 | 0.04 | 0.18 | 0.12 | 0.1190 | AA213799 | LOC284120 | hypothetical LOC284120 |
| 1566111_at | 1.52 | 0.0587 | −1.98 | 5.09 | 7.73 | 0.02 | 0.04 | 0.18 | 0.10 | 0.0783 | AL832442 | na | MRNA; cDNA |
| | | | | | | | | | | | | | DKFZp762L214 (from |
| | | | | | | | | | | | | | clone DKFZp762L214) |
| 1562562_at | 1.52 | 0.1049 | −1.69 | 4.10 | 6.22 | 0.02 | 0.03 | 0.18 | 0.15 | 0.2478 | AK098078 | FLJ40759 | hypothetical gene |
| | | | | | | | | | | | | | supported by AK098078 |
| 1565859_at | 1.52 | 0.0235 | −2.44 | 2.96 | 4.49 | 0.01 | 0.02 | 0.18 | 0.10 | 0.0628 | BM918074 | LOC388796 | hypothetical LOC388796 |
| 221944_at | 1.51 | 0.3904 | −0.88 | 55.45 | 84.01 | 0.26 | 0.39 | 0.18 | 0.19 | 0.3588 | N56912 | FLJ42627 | hypothetical protein |
| | | | | | | | | | | | | | LOC645644 |
| 235974_at | 1.51 | 0.1312 | −1.56 | 8.35 | 12.64 | 0.04 | 0.06 | 0.18 | 0.10 | 0.0743 | AI857698 | EXOC4 | Exocyst complex |
| | | | | | | | | | | | | | component 4 |
| 224165_s_at | 1.51 | 0.1933 | −1.35 | 4.26 | 6.46 | 0.02 | 0.03 | 0.18 | 0.13 | 0.1801 | AY014282 | IQCH | IQ motif containing H |
| 214038_at | 1.51 | 0.3814 | −0.89 | 12.37 | 18.71 | 0.06 | 0.09 | 0.18 | 0.17 | 0.2893 | AI984980 | CCL8 | wr88g11.x1 |
| | | | | | | | | | | | | | NCI_CGAP_Kid11 Homo |
| | | | | | | | | | | | | | sapiens cDNA clone |
| | | | | | | | | | | | | | IMAGE: 2494820 3′ |
| | | | | | | | | | | | | | similar to |
| | | | | | | | | | | | | | SW: MCP2_HUMAN |
| | | | | | | | | | | | | | P80075 MONOCYTE |
| | | | | | | | | | | | | | CHEMOTACTIC |
| | | | | | | | | | | | | | PROTEIN 2 |
| | | | | | | | | | | | | | PRECURSOR; mRNA |
| | | | | | | | | | | | | | sequence. |
| 240539_at | 1.51 | 0.0068 | −2.93 | 6.04 | 9.14 | 0.03 | 0.04 | 0.18 | 0.09 | 0.0367 | AI684551 | AUTS2 | Autism susceptibility |
| | | | | | | | | | | | | | candidate 2 |
| 200635_s_at | 1.51 | 0.0786 | −1.84 | 9.37 | 14.18 | 0.04 | 0.07 | 0.18 | 0.11 | 0.0900 | AU145351 | PTPRF | Protein tyrosine |
| | | | | | | | | | | | | | phosphatase, receptor type, F |
| 241695_s_at | 1.51 | 0.0719 | −1.87 | 6.69 | 10.12 | 0.03 | 0.05 | 0.18 | 0.12 | 0.1295 | AA648986 | KLHL6 | Kelch-like 6 (Drosophila) |
| 240953_at | 1.51 | 0.4992 | −0.69 | 5.41 | 8.19 | 0.02 | 0.04 | 0.18 | 0.18 | 0.3242 | AI821136 | ACPP | Acid phosphatase, prostate |
| 227498_at | 1.51 | 0.3286 | −1.01 | 8.04 | 12.15 | 0.04 | 0.06 | 0.18 | 0.19 | 0.3660 | AI480314 | 227498_at | CDNA FLJ11723 fis, |
| | | | | | | | | | | | | | clone HEMBA1005314 |
| 220047_at | 1.51 | 0.1672 | −1.43 | 10.75 | 16.24 | 0.05 | 0.08 | 0.18 | 0.12 | 0.1426 | NM_012240 | SIRT4 | Sirtuin (silent mating type |
| | | | | | | | | | | | | | information regulation 2 |
| | | | | | | | | | | | | | homolog) 4 (S. cerevisiae) |
| 241453_at | 1.51 | 0.2449 | −1.20 | 5.91 | 8.92 | 0.03 | 0.04 | 0.18 | 0.16 | 0.2688 | AA912743 | PTK2 | PTK2 protein tyrosine |
| | | | | | | | | | | | | | kinase 2 |
| 244693_at | 1.51 | 0.1418 | −1.52 | 11.18 | 16.87 | 0.05 | 0.08 | 0.18 | 0.13 | 0.1656 | BF110113 | USP54 | Ubiquitin specific |
| | | | | | | | | | | | | | peptidase 54 |
| 236219_at | 1.51 | 0.2827 | −1.10 | 5.72 | 8.63 | 0.03 | 0.04 | 0.18 | 0.10 | 0.0635 | AI452512 | TMEM20 | Transmembrane protein 20 |
| 207738_s_at | 1.51 | 0.0157 | −2.60 | 25.06 | 37.79 | 0.12 | 0.18 | 0.18 | 0.07 | 0.0092 | NM_013436 | NCKAP1 | NCK-associated protein 1 |
| 239548_at | 1.51 | 0.0201 | −2.47 | 4.15 | 6.26 | 0.02 | 0.03 | 0.18 | 0.10 | 0.0837 | AW001754 | NEGR1 | Neuronal growth regulator 1 |
| 1555815_a_at | 1.51 | 0.0666 | −1.91 | 5.01 | 7.55 | 0.02 | 0.04 | 0.18 | 0.16 | 0.2396 | AL136564 | L3MBTL2 | L(3)mbt-like 2 |
| | | | | | | | | | | | | | (Drosophila) |
| 236062_at | 1.51 | 0.1274 | −1.57 | 6.49 | 9.79 | 0.03 | 0.05 | 0.18 | 0.13 | 0.1748 | AI742722 | UBE2E1 | Ubiquitin-conjugating |
| | | | | | | | | | | | | | enzyme E2E 1 (UBC4/5 |
| | | | | | | | | | | | | | homolog, yeast) |
| 1556439_at | 1.51 | 0.0060 | −2.98 | 4.09 | 6.17 | 0.02 | 0.03 | 0.18 | 0.08 | 0.0218 | AL832163 | LOC441376 | AARD protein |
| 233660_at | 1.51 | 0.0603 | −1.96 | 15.34 | 23.12 | 0.07 | 0.11 | 0.18 | 0.08 | 0.0262 | BG540685 | EHD4 | EH-domain containing 4 |
| 208365_s_at | 1.51 | 0.0994 | −1.70 | 4.75 | 7.16 | 0.02 | 0.03 | 0.18 | 0.12 | 0.1167 | NM_001004056 | GRK4 | G protein-coupled receptor |
| | | | | | | | | | | | | | kinase 4 |
| 223143_s_at | 1.51 | 0.0138 | −2.63 | 4.41 | 6.64 | 0.02 | 0.03 | 0.18 | 0.10 | 0.0683 | AI742378 | C6orf166 | Chromosome 6 open |
| | | | | | | | | | | | | | reading frame 166 |
| 1570156_s_at | 1.51 | 0.0491 | −2.10 | 5.48 | 8.26 | 0.03 | 0.04 | 0.18 | 0.12 | 0.1450 | BC015906 | FMN1 | Homo sapiens formin 1, |
| | | | | | | | | | | | | | mRNA (cDNA clone |
| | | | | | | | | | | | | | IMAGE: 3922558), **** |
| | | | | | | | | | | | | | WARNING: chimeric |
| | | | | | | | | | | | | | clone ****. |
| 226439_s_at | 1.51 | 0.1745 | −1.40 | 13.66 | 20.57 | 0.06 | 0.10 | 0.18 | 0.11 | 0.1284 | AI246710 | NBEA | Neurobeachin |
| 239269_at | 1.51 | 0.1464 | −1.49 | 4.90 | 7.39 | 0.02 | 0.03 | 0.18 | 0.10 | 0.0793 | AW449577 | TncRNA | Transcribed locus |
| 244734_at | 1.51 | 0.0890 | −1.79 | 6.10 | 9.18 | 0.03 | 0.04 | 0.18 | 0.17 | 0.3019 | W45568 | MTHFSD | Methenyltetrahydrofolate |
| | | | | | | | | | | | | | synthetase domain |
| | | | | | | | | | | | | | containing |
| 244042_x_at | 1.50 | 0.0850 | −1.79 | 14.49 | 21.80 | 0.07 | 0.10 | 0.18 | 0.10 | 0.0641 | AA883831 | LOC651466 | am21g03.s1 |
| | | | | | | | | | | | | | Soares_NFL_T_GBC_S1 |
| | | | | | | | | | | | | | Homo sapiens cDNA clone |
| | | | | | | | | | | | | | IMAGE: 1467508 3′, |
| | | | | | | | | | | | | | mRNA sequence. |
| 242798_at | 1.50 | 0.0457 | −2.09 | 6.40 | 9.64 | 0.03 | 0.04 | 0.18 | 0.13 | 0.1648 | AI247368 | 242798_at | Transcribed locus |
| 206506_s_at | 1.50 | 0.4341 | −0.80 | 16.38 | 24.64 | 0.08 | 0.11 | 0.18 | 0.11 | 0.1111 | NM_003599 | SUPT3H | Suppressor of Ty 3 |
| | | | | | | | | | | | | | homolog (S. cerevisiae) |
| 223623_at | 1.50 | 0.1899 | −1.35 | 15.17 | 22.80 | 0.07 | 0.11 | 0.18 | 0.12 | 0.1617 | AF325503 | ECRG4 | esophageal cancer related |
| | | | | | | | | | | | | | gene 4 protein |
| 233210_at | 1.50 | 0.1571 | −1.47 | 30.20 | 45.37 | 0.14 | 0.21 | 0.18 | 0.14 | 0.2214 | AK022182 | FLJ12120 | hypothetical LOC388439 |
| 205819_at | −1.50 | 0.5766 | 0.57 | 17.24 | 11.49 | 0.08 | 0.05 | −0.18 | 0.18 | 0.2998 | NM_006770 | MARCO | Macrophage receptor with |
| | | | | | | | | | | | | | collagenous structure |
| 242957_at | −1.50 | 0.0172 | 2.57 | 363.41 | 241.59 | 1.67 | 1.12 | −0.18 | 0.10 | 0.0598 | AI862096 | VWCE | Von Willebrand factor C |
| | | | | | | | | | | | | | and EGF domains |
| 235332_at | −1.50 | 0.0356 | 2.21 | 12.72 | 8.46 | 0.06 | 0.04 | −0.18 | 0.11 | 0.0940 | AW501360 | FAM22B | Family with sequence |
| | | | | | | | | | | | | | similarity 22, member B |
| 1569095_at | −1.50 | 0.1604 | 1.44 | 149.91 | 99.61 | 0.69 | 0.46 | −0.18 | 0.09 | 0.0404 | BC016366 | LOC731424 | Homo sapiens, clone |
| | | | | | | | | | | | | | IMAGE: 4133286, mRNA |
| 1557206_at | −1.51 | 0.4060 | 0.84 | 7.21 | 4.78 | 0.03 | 0.02 | −0.18 | 0.13 | 0.1537 | BC035159 | FLJ35848 | hypothetical protein |
| | | | | | | | | | | | | | FLJ35848 |
| 1557122_s_at | −1.51 | 0.0789 | 1.82 | 11.39 | 7.56 | 0.05 | 0.04 | −0.18 | 0.11 | 0.1094 | BC036592 | GABRB2 | CDNA clone |
| | | | | | | | | | | | | | IMAGE: 4814184 |
| 231618_s_at | −1.51 | 0.1021 | 1.69 | 14.61 | 9.70 | 0.07 | 0.05 | −0.18 | 0.13 | 0.1802 | AI221329 | SUNC1 | Sad1 and UNC84 domain |
| | | | | | | | | | | | | | containing 1 |
| 232404_at | −1.51 | 0.0158 | 2.57 | 7.85 | 5.21 | 0.04 | 0.02 | −0.18 | 0.12 | 0.1265 | AB033028 | SHROOM4 | KIAA1202 protein |
| 233462_at | −1.51 | 0.0753 | 1.85 | 11.48 | 7.62 | 0.05 | 0.04 | −0.18 | 0.08 | 0.0266 | AL137747 | FLJ40244 | hypothetical protein |
| | | | | | | | | | | | | | FLJ40244 |
| 223791_at | −1.51 | 0.1309 | 1.56 | 8.33 | 5.52 | 0.04 | 0.03 | −0.18 | 0.13 | 0.1683 | BC002886 | FAM27A | Family with sequence |
| | | | | | | | | | | | | | similarity 27, member A |
| 220795_s_at | −1.51 | 0.1056 | 1.67 | 50.43 | 33.39 | 0.23 | 0.16 | −0.18 | 0.14 | 0.2084 | NM_020836 | KIAA1446 | likely ortholog of rat brain- |
| | | | | | | | | | | | | | enriched guanylate kinase- |
| | | | | | | | | | | | | | associated protein |
| 239944_at | −1.51 | 0.0848 | 1.82 | 195.63 | 129.48 | 0.90 | 0.60 | −0.18 | 0.11 | 0.1125 | AA431379 | DKFZP686P18101 | similar to TFIIH basal |
| | | | | | | | | | | | | | transcription factor |
| | | | | | | | | | | | | | complex p44 subunit |
| | | | | | | | | | | | | | (Basic transcription factor |
| | | | | | | | | | | | | | 2 44 kDa subunit) (BTF2- |
| | | | | | | | | | | | | | p44) (General transcription |
| | | | | | | | | | | | | | factor IIH polypeptide 2) |
| 211734_s_at | −1.51 | 0.0017 | 3.48 | 905.80 | 599.35 | 4.17 | 2.78 | −0.18 | 0.05 | 0.0004 | BC005912 | FCER1A | Fc fragment of IgE, high |
| | | | | | | | | | | | | | affinity I, receptor for; |
| | | | | | | | | | | | | | alpha polypeptide |
| 211891_s_at | −1.51 | 0.2321 | 1.23 | 6.45 | 4.27 | 0.03 | 0.02 | −0.18 | 0.19 | 0.3333 | AB042199 | ARHGEF4 | Rho guanine nucleotide |
| | | | | | | | | | | | | | exchange factor (GEF) 4 |
| 1557136_at | −1.51 | 0.2201 | 1.25 | 7.48 | 4.95 | 0.03 | 0.02 | −0.18 | 0.17 | 0.2898 | BG059633 | ATP13A4 | ATPase type 13A4 |
| 236618_at | −1.51 | 0.4384 | 0.79 | 11.63 | 7.69 | 0.05 | 0.04 | −0.18 | 0.18 | 0.3296 | AW300370 | C20orf132 | Chromosome 20 open |
| | | | | | | | | | | | | | reading frame 132 |
| 209212_s_at | −1.51 | 0.0634 | 1.94 | 15.51 | 10.25 | 0.07 | 0.05 | −0.18 | 0.11 | 0.0874 | AB030824 | KLF5 | Kruppel-like factor 5 |
| | | | | | | | | | | | | | (intestinal) |
| 208025_s_at | −1.51 | 0.0115 | 2.80 | 6.70 | 4.43 | 0.03 | 0.02 | −0.18 | 0.09 | 0.0336 | NM_003483 | HMGA2 | High mobility group AT- |
| | | | | | | | | | | | | | hook 2 |
| 209891_at | −1.52 | 0.2555 | 1.16 | 7.47 | 4.93 | 0.03 | 0.02 | −0.18 | 0.13 | 0.1640 | AF225416 | SPBC25 | Spindle pole body |
| | | | | | | | | | | | | | component 25 homolog (S. cerevisiae) |
| 239718_at | −1.52 | 0.0619 | 1.96 | 6.97 | 4.60 | 0.03 | 0.02 | −0.18 | 0.14 | 0.1847 | R42552 | LOC654342 | yg01a09.s1 Soares infant |
| | | | | | | | | | | | | | brain 1NIB Homo sapiens |
| | | | | | | | | | | | | | cDNA clone |
| | | | | | | | | | | | | | IMAGE: 30807 3′, mRNA |
| | | | | | | | | | | | | | sequence. |
| 228375_at | −1.52 | 0.3992 | 0.86 | 13.22 | 8.72 | 0.06 | 0.04 | −0.18 | 0.16 | 0.2668 | BE221674 | IGSF11 | Immunoglobulin |
| | | | | | | | | | | | | | superfamily, member 11 |
| 214106_s_at | −1.52 | 0.0912 | 1.75 | 8.67 | 5.71 | 0.04 | 0.03 | −0.18 | 0.12 | 0.1285 | AI762113 | GMDS | GDP-mannose 4,6- |
| | | | | | | | | | | | | | dehydratase |
| 223975_at | −1.52 | 0.0696 | 1.89 | 19.73 | 12.99 | 0.09 | 0.06 | −0.18 | 0.11 | 0.0855 | BC005014 | SPRYD5 | SPRY domain containing 5 |
| 1557051_s_at | −1.52 | 0.0182 | 2.55 | 395.73 | 260.52 | 1.82 | 1.21 | −0.18 | 0.07 | 0.0114 | CA448125 | 1557051_s_at | Homo sapiens, clone |
| | | | | | | | | | | | | | IMAGE: 5019307, mRNA |
| 222768_s_at | −1.52 | 0.0317 | 2.26 | 32.23 | 21.21 | 0.15 | 0.10 | −0.18 | 0.09 | 0.0442 | BE897074 | CGI-09 | CGI-09 protein |
| 218985_at | −1.52 | 0.5387 | 0.62 | 20.88 | 13.74 | 0.10 | 0.06 | −0.18 | 0.12 | 0.1364 | NM_014580 | SLC2A8 | Solute carrier family 2, |
| | | | | | | | | | | | | | (facilitated glucose |
| | | | | | | | | | | | | | transporter) member 8 |
| 219855_at | −1.52 | 0.1576 | 1.45 | 14.78 | 9.73 | 0.07 | 0.05 | −0.18 | 0.11 | 0.0867 | NM_018159 | NUDT11 | Nudix (nucleoside |
| | | | | | | | | | | | | | diphosphate linked moiety |
| | | | | | | | | | | | | | X)-type motif 11 |
| 214639_s_at | −1.52 | 0.0043 | 3.11 | 46.76 | 30.77 | 0.22 | 0.14 | −0.18 | 0.08 | 0.0173 | S79910 | HOXA1 | Homeobox A1 |
| 235763_at | −1.52 | 0.4124 | 0.83 | 11.41 | 7.51 | 0.05 | 0.03 | −0.18 | 0.20 | 0.3511 | AA001450 | SLC44A5 | Solute carrier family 44, |
| | | | | | | | | | | | | | member 5 |
| 239115_at | −1.52 | 0.0154 | 2.58 | 7.12 | 4.68 | 0.03 | 0.02 | −0.18 | 0.14 | 0.1631 | AA670271 | na | af25e10.s1 |
| | | | | | | | | | | | | | Soares_total_fetus_Nb2HF8_9w |
| | | | | | | | | | | | | | Homo sapiens |
| | | | | | | | | | | | | | cDNA clone |
| | | | | | | | | | | | | | IMAGE: 1032714 3′, |
| | | | | | | | | | | | | | mRNA sequence. |
| 236928_at | −1.52 | 0.0338 | 2.23 | 14.69 | 9.66 | 0.07 | 0.04 | −0.18 | 0.14 | 0.1750 | AA830326 | LOC644173 | hypothetical protein |
| | | | | | | | | | | | | | LOC644173 |
| 214465_at | −1.52 | 0.0611 | 1.96 | 53.06 | 34.87 | 0.24 | 0.16 | −0.18 | 0.09 | 0.0492 | NM_000608 | ORM2 | Orosomucoid 2 |
| 230218_at | −1.52 | 0.3064 | 1.04 | 12.01 | 7.89 | 0.06 | 0.04 | −0.18 | 0.14 | 0.1989 | BF476403 | HIC1 | Transcribed locus |
| 237538_at | −1.52 | 0.3013 | 1.05 | 6.50 | 4.27 | 0.03 | 0.02 | −0.18 | 0.17 | 0.2853 | BE552359 | RSAD2 | Radical S-adenosyl |
| | | | | | | | | | | | | | methionine domain |
| | | | | | | | | | | | | | containing 2 |
| 243434_at | −1.52 | 0.0134 | 2.65 | 6.88 | 4.52 | 0.03 | 0.02 | −0.18 | 0.11 | 0.0794 | BE674989 | C1orf75 | Chromosome 1 open |
| | | | | | | | | | | | | | reading frame 75 |
| 243359_at | −1.52 | 0.0316 | 2.27 | 6.50 | 4.26 | 0.03 | 0.02 | −0.18 | 0.09 | 0.0439 | AI701857 | 243359_at | Transcribed locus |
| 241541_at | −1.52 | 0.2930 | 1.08 | 6.16 | 4.04 | 0.03 | 0.02 | −0.18 | 0.17 | 0.2675 | AW511227 | MIB2 | Mindbomb homolog 2 |
| | | | | | | | | | | | | | (Drosophila) |
| 214296_x_at | −1.52 | 0.0012 | 3.67 | 11.88 | 7.80 | 0.05 | 0.04 | −0.18 | 0.09 | 0.0437 | AV721013 | C19or136 | Chromosome 19 open |
| | | | | | | | | | | | | | reading frame 36 |
| 214457_at | −1.52 | 0.0010 | 3.68 | 77.89 | 51.10 | 0.36 | 0.24 | −0.18 | 0.06 | 0.0023 | NM_006735 | HOXA2 | Homeobox A2 |
| 239647_at | −1.52 | 0.0077 | 3.00 | 233.18 | 152.97 | 1.07 | 0.71 | −0.18 | 0.09 | 0.0390 | AA677272 | CHST13 | Carbohydrate (chondroitin |
| | | | | | | | | | | | | | 4) sulfotransferase 13 |
| 209291_at | −1.53 | 0.0483 | 2.07 | 8.16 | 5.35 | 0.04 | 0.02 | −0.18 | 0.12 | 0.1147 | AW157094 | ID4 | Inhibitor of DNA binding |
| | | | | | | | | | | | | | 4, dominant negative |
| | | | | | | | | | | | | | helix-loop-helix protein |
| 230277_at | −1.53 | 0.4493 | 0.77 | 9.97 | 6.53 | 0.05 | 0.03 | −0.18 | 0.20 | 0.3391 | AI806865 | ZNF655 | Zinc finger protein 655 |
| 243334_at | −1.53 | 0.0514 | 2.03 | 17.93 | 11.75 | 0.08 | 0.05 | −0.18 | 0.10 | 0.0585 | BF224050 | CACNA1D | Calcium channel, voltage- |
| | | | | | | | | | | | | | dependent, L type, alpha |
| | | | | | | | | | | | | | 1D subunit |
| 236348_at | −1.53 | 0.0847 | 1.79 | 19.48 | 12.75 | 0.09 | 0.06 | −0.18 | 0.12 | 0.1194 | H48531 | KCNH2 | Potassium voltage-gated |
| | | | | | | | | | | | | | channel, subfamily H (eag- |
| | | | | | | | | | | | | | related), member 2 |
| 220824_at | −1.53 | 0.0220 | 2.43 | 10.18 | 6.66 | 0.05 | 0.03 | −0.18 | 0.09 | 0.0390 | NM_182628 | CCDC37 | gb: NM_017674.1 |
| | | | | | | | | | | | | | /DB_XREF = gi: 8923119 |
| | | | | | | | | | | | | | /GEN = FLJ20123 |
| | | | | | | | | | | | | | /FEA = FLmRNA /CNT = 3 |
| | | | | | | | | | | | | | /TID = Hs.272232.0 |
| | | | | | | | | | | | | | /TIER = FL /STK = 0 |
| | | | | | | | | | | | | | /UG = Hs.272232 |
| | | | | | | | | | | | | | /LL = 54824 /DEF = Homo |
| | | | | | | | | | | | | | sapiens hypothetical |
| | | | | | | | | | | | | | protein FLJ20123 |
| | | | | | | | | | | | | | (FLJ20123), mRNA. |
| | | | | | | | | | | | | | /PROD = hypothetical |
| | | | | | | | | | | | | | protein FLJ20123 |
| | | | | | | | | | | | | | /FL = gb: NM_017674.1 |
| 223994_s_at | −1.53 | 0.1535 | 1.47 | 7.30 | 4.77 | 0.03 | 0.02 | −0.18 | 0.15 | 0.2246 | BC000154 | SLC12A9 | Solute carrier family 12 |
| | | | | | | | | | | | | | (potassium/chloride |
| | | | | | | | | | | | | | transporters), member 9 |
| 238209_at | −1.53 | 0.1158 | 1.65 | 7.75 | 5.07 | 0.04 | 0.02 | −0.18 | 0.20 | 0.3675 | AW005925 | C9orf164 | Transcribed locus |
| 244865_at | −1.53 | 0.0825 | 1.80 | 6.52 | 4.26 | 0.03 | 0.02 | −0.18 | 0.14 | 0.1777 | AI420119 | HAX1 | HCLS1 associated protein |
| | | | | | | | | | | | | | X-1 |
| 206697_s_at | −1.53 | 0.0150 | 2.59 | 547.02 | 357.40 | 2.52 | 1.66 | −0.18 | 0.08 | 0.0146 | NM_005143 | HP | Haptoglobin |
| 206234_s_at | −1.53 | 0.0399 | 2.17 | 7.79 | 5.09 | 0.04 | 0.02 | −0.19 | 0.10 | 0.0654 | NM_016155 | MMP17 | Matrix metallopeptidase |
| | | | | | | | | | | | | | 17 (membrane-inserted) |
| 229954_at | −1.53 | 0.4205 | 0.82 | 12.44 | 8.12 | 0.06 | 0.04 | −0.19 | 0.18 | 0.2988 | AI025415 | CHDH | Transcribed locus |
| 235570_at | −1.53 | 0.1072 | 1.67 | 10.21 | 6.66 | 0.05 | 0.03 | −0.19 | 0.13 | 0.1477 | AW298235 | RBMS3 | Transcribed locus |
| 233273_at | −1.53 | 0.0481 | 2.07 | 7.24 | 4.72 | 0.03 | 0.02 | −0.19 | 0.17 | 0.2612 | AU146834 | PBX1 | AU146834 HEMBB1 |
| | | | | | | | | | | | | | Homo sapiens cDNA clone |
| | | | | | | | | | | | | | HEMBB1001635 3′, |
| | | | | | | | | | | | | | mRNA sequence. |
| 214575_s_at | −1.54 | 0.1666 | 1.42 | 21.29 | 13.87 | 0.10 | 0.06 | −0.19 | 0.11 | 0.0801 | NM_001700 | AZU1 | Azurocidin 1 (cationic |
| | | | | | | | | | | | | | antimicrobial protein 37) |
| 224803_s_at | −1.54 | 0.2155 | 1.27 | 6.97 | 4.54 | 0.03 | 0.02 | −0.19 | 0.16 | 0.2332 | AK024040 | LOC148413 | hypothetical protein |
| | | | | | | | | | | | | | LOC148413 |
| 240031_at | −1.54 | 0.2201 | 1.25 | 13.13 | 8.55 | 0.06 | 0.04 | −0.19 | 0.13 | 0.1522 | AA994467 | MSRA | Methionine sulfoxide |
| | | | | | | | | | | | | | reductase A |
| 204033_at | −1.54 | 0.0717 | 1.88 | 10.45 | 6.80 | 0.05 | 0.03 | −0.19 | 0.14 | 0.1666 | NM_004237 | TRIP13 | Thyroid hormone receptor |
| | | | | | | | | | | | | | interactor 13 |
| 203757_s_at | −1.54 | 0.1230 | 1.59 | 71.12 | 46.21 | 0.33 | 0.21 | −0.19 | 0.15 | 0.2034 | BC005008 | CEACAM6 | Carcinoembryonic antigen- |
| | | | | | | | | | | | | | related cell adhesion |
| | | | | | | | | | | | | | molecule 6 (non-specific |
| | | | | | | | | | | | | | cross reacting antigen) |
| 211483_x_at | −1.54 | 0.1330 | 1.56 | 7.59 | 4.93 | 0.03 | 0.02 | −0.19 | 0.18 | 0.2884 | AF081924 | CAMK2B | Calcium/calmodulin- |
| | | | | | | | | | | | | | dependent protein kinase |
| | | | | | | | | | | | | | (CaM kinase) II beta |
| 1555758_a_at | −1.54 | 0.0341 | 2.23 | 8.23 | 5.34 | 0.04 | 0.02 | −0.19 | 0.12 | 0.0973 | AF213040 | CDKN3 | Cyclin-dependent kinase |
| | | | | | | | | | | | | | inhibitor 3 (CDK2- |
| | | | | | | | | | | | | | associated dual specificity |
| | | | | | | | | | | | | | phosphatase) |
| 243097_x_at | −1.54 | 0.0231 | 2.41 | 8.60 | 5.59 | 0.04 | 0.03 | −0.19 | 0.09 | 0.0352 | R55769 | na | yg89e01.s1 Soares infant |
| | | | | | | | | | | | | | brain 1NIB Homo sapiens |
| | | | | | | | | | | | | | cDNA clone |
| | | | | | | | | | | | | | IMAGE: 40625 3′, mRNA |
| | | | | | | | | | | | | | sequence. |
| 1564568_at | −1.54 | 0.3396 | 0.97 | 8.91 | 5.79 | 0.04 | 0.03 | −0.19 | 0.17 | 0.2779 | AL050168 | ELL | Elongation factor RNA |
| | | | | | | | | | | | | | polymerase II |
| 1557022_at | −1.54 | 0.3320 | 0.99 | 6.45 | 4.18 | 0.03 | 0.02 | −0.19 | 0.21 | 0.3648 | BC041900 | 1557022_at | CDNA clone |
| | | | | | | | | | | | | | IMAGE: 5298883 |
| 240744_at | −1.54 | 0.4730 | 0.73 | 71.70 | 46.54 | 0.33 | 0.22 | −0.19 | 0.19 | 0.3194 | AW184014 | CPAS | Carboxypeptidase A5 |
| 233771_at | −1.54 | 0.1928 | 1.34 | 7.12 | 4.62 | 0.03 | 0.02 | −0.19 | 0.15 | 0.1956 | AU156625 | TRIO | AU156625 PLACE1 |
| | | | | | | | | | | | | | Homo sapiens cDNA clone |
| | | | | | | | | | | | | | PLACE1003936 3′, |
| | | | | | | | | | | | | | mRNA sequence. |
| 1560175_at | −1.54 | 0.5200 | 0.65 | 7.88 | 5.11 | 0.04 | 0.02 | −0.19 | 0.16 | 0.2457 | AK057583 | PPP4R1L | Protein phosphatase 4, |
| | | | | | | | | | | | | | regulatory subunit 1-like |
| 227530_at | −1.54 | 0.0528 | 2.02 | 63.10 | 40.90 | 0.29 | 0.19 | −0.19 | 0.10 | 0.0464 | BF511276 | AKAP12 | A kinase (PRKA) anchor |
| | | | | | | | | | | | | | protein (gravin) 12 |
| 215458_s_at | −1.54 | 0.0007 | 3.82 | 10.33 | 6.69 | 0.05 | 0.03 | −0.19 | 0.09 | 0.0284 | AF199364 | SMURF1 | SMAD specific E3 |
| | | | | | | | | | | | | | ubiquitin protein ligase 1 |
| 222869_s_at | −1.54 | 0.1196 | 1.61 | 34.89 | 22.59 | 0.16 | 0.10 | −0.19 | 0.12 | 0.1093 | AI669235 | ELAC1 | ElaC homolog 1 (E. coli) |
| 220020_at | −1.54 | 0.2286 | 1.23 | 9.57 | 6.20 | 0.04 | 0.03 | −0.19 | 0.18 | 0.2704 | NM_022098 | LOC63929 | hypothetical protein |
| | | | | | | | | | | | | | LOC63929 |
| 236049_at | −1.55 | 0.0452 | 2.10 | 7.82 | 5.06 | 0.04 | 0.02 | −0.19 | 0.13 | 0.1385 | AI277101 | WDR90 | Hypothetical protein |
| | | | | | | | | | | | | | KIAA1924 |
| 224173_s_at | −1.55 | 0.2361 | 1.21 | 23.35 | 15.10 | 0.11 | 0.07 | −0.19 | 0.11 | 0.0937 | NM_145212 | MRPL30 | Mitochondrial ribosomal |
| | | | | | | | | | | | | | protein L30 |
| 218725_at | −1.55 | 0.0217 | 2.47 | 38.56 | 24.92 | 0.18 | 0.12 | −0.19 | 0.06 | 0.0019 | NM_024698 | SLC25A22 | Solute carrier family 25 |
| | | | | | | | | | | | | | (mitochondrial carrier: |
| | | | | | | | | | | | | | glutamate), member 22 |
| 219978_s_at | −1.55 | 0.0181 | 2.51 | 40.21 | 25.99 | 0.19 | 0.12 | −0.19 | 0.08 | 0.0177 | NM_018454 | NUSAP1 | Nucleolar and spindle |
| | | | | | | | | | | | | | associated protein 1 |
| 211657_at | −1.55 | 0.3345 | 0.98 | 140.11 | 90.53 | 0.65 | 0.42 | −0.19 | 0.14 | 0.1671 | M18728 | CEACAM6 | Carcinoembryonic antigen- |
| | | | | | | | | | | | | | related cell adhesion |
| | | | | | | | | | | | | | molecule 6 (non-specific |
| | | | | | | | | | | | | | cross reacting antigen) |
| 240199_x_at | −1.55 | 0.1470 | 1.50 | 7.18 | 4.63 | 0.03 | 0.02 | −0.19 | 0.16 | 0.2111 | AI016940 | ZNF345 | Zinc finger protein 345 |
| 205445_at | −1.55 | 0.0469 | 2.08 | 11.95 | 7.71 | 0.06 | 0.04 | −0.19 | 0.11 | 0.0900 | NM_000948 | PRL | Prolactin |
| 230136_at | −1.55 | 0.0562 | 1.99 | 38.42 | 24.79 | 0.18 | 0.12 | −0.19 | 0.08 | 0.0177 | AI573252 | LOC400099 | hypothetical gene |
| | | | | | | | | | | | | | supported by BC024195 |
| 222134_at | −1.55 | 0.0395 | 2.16 | 15.89 | 10.24 | 0.07 | 0.05 | −0.19 | 0.13 | 0.1417 | AL050350 | DDO | Human DNA sequence |
| | | | | | | | | | | | | | from clone RP1-261K5 on |
| | | | | | | | | | | | | | chromosome 6q21-22.1 |
| | | | | | | | | | | | | | Contains the 3′ end of the |
| | | | | | | | | | | | | | SLC22A16 gene for solute |
| | | | | | | | | | | | | | carrier family 22 (organic |
| | | | | | | | | | | | | | cation transporter) member |
| | | | | | | | | | | | | | 16, the DDO gene for D- |
| | | | | | | | | | | | | | aspartate oxidase, the 5′ |
| | | | | | | | | | | | | | part of a novel gene and |
| | | | | | | | | | | | | | two CpG islands, complete |
| | | | | | | | | | | | | | sequence. |
| 232590_at | −1.55 | 0.0290 | 2.30 | 10.21 | 6.58 | 0.05 | 0.03 | −0.19 | 0.10 | 0.0442 | AK025919 | HADHA | Hydroxyacyl-Coenzyme A |
| | | | | | | | | | | | | | dehydrogenase/3-ketoacyl- |
| | | | | | | | | | | | | | Coenzyme A |
| | | | | | | | | | | | | | thiolase/enoyl-Coenzyme |
| | | | | | | | | | | | | | A hydratase (trifunctional |
| | | | | | | | | | | | | | protein), alpha subunit |
| 233607_at | −1.55 | 0.3061 | 1.05 | 6.39 | 4.11 | 0.03 | 0.02 | −0.19 | 0.16 | 0.2430 | AU145679 | BICC1 | Bicaudal C homolog 1 |
| | | | | | | | | | | | | | (Drosophila) |
| 213284_at | −1.55 | 0.2925 | 1.08 | 8.17 | 5.26 | 0.04 | 0.02 | −0.19 | 0.22 | 0.3895 | BG482928 | ZFP36L1 | Zinc finger protein 36, |
| | | | | | | | | | | | | | C3H type-like 1 |
| 1564357_at | −1.56 | 0.2466 | 1.18 | 6.83 | 4.39 | 0.03 | 0.02 | −0.19 | 0.14 | 0.1485 | AK057860 | C14orf29 | Homo sapiens cDNA |
| | | | | | | | | | | | | | FLJ25131 fis, clone |
| | | | | | | | | | | | | | CBR06643. |
| 232605_s_at | −1.56 | 0.0893 | 1.76 | 6.95 | 4.47 | 0.03 | 0.02 | −0.19 | 0.14 | 0.1525 | AA226334 | LOC646871 | hypothetical protein |
| | | | | | | | | | | | | | LOC646871 |
| 1553605_a_at | −1.56 | 0.0632 | 1.94 | 34.32 | 22.02 | 0.16 | 0.10 | −0.19 | 0.13 | 0.1243 | NM_152701 | ABCA13 | ATP-binding cassette, sub- |
| | | | | | | | | | | | | | family A (ABC1), member |
| | | | | | | | | | | | | | 13 |
| 237461_at | −1.56 | 0.3181 | 1.02 | 27.23 | 17.46 | 0.13 | 0.08 | −0.19 | 0.15 | 0.2017 | AA565499 | NALP7 | NACHT, leucine rich |
| | | | | | | | | | | | | | repeat and PYD containing 7 |
| 1563407_x_at | −1.56 | 0.1563 | 1.47 | 9.03 | 5.78 | 0.04 | 0.03 | −0.19 | 0.18 | 0.2773 | BC042846 | ATP4B | ATPase, H+/K+ |
| | | | | | | | | | | | | | exchanging, beta |
| | | | | | | | | | | | | | polypeptide |
| 216048_s_at | −1.56 | 0.0920 | 1.74 | 7.48 | 4.79 | 0.03 | 0.02 | −0.19 | 0.13 | 0.1234 | AK023621 | RHOBTB3 | Rho-related BTB domain |
| | | | | | | | | | | | | | containing 3 |
| 238466_at | −1.56 | 0.0524 | 2.05 | 7.27 | 4.65 | 0.03 | 0.02 | −0.19 | 0.13 | 0.1229 | R43486 | ZNF91 | Transcribed locus |
| 1553063_at | −1.56 | 0.0019 | 3.42 | 21.21 | 13.58 | 0.10 | 0.06 | −0.19 | 0.08 | 0.0122 | NM_080819 | GPR78 | G protein-coupled receptor |
| | | | | | | | | | | | | | 78 |
| 210640_s_at | −1.56 | 0.2608 | 1.15 | 29.88 | 19.11 | 0.14 | 0.09 | −0.19 | 0.18 | 0.2693 | U63917 | GPR30 | G protein-coupled receptor |
| | | | | | | | | | | | | | 30 |
| 212084_at | −1.56 | 0.1397 | 1.53 | 9.82 | 6.28 | 0.05 | 0.03 | −0.19 | 0.14 | 0.1496 | AV759552 | TEX261 | Testis expressed sequence |
| | | | | | | | | | | | | | 261 |
| 243590_at | −1.56 | 0.0863 | 1.78 | 9.36 | 5.99 | 0.04 | 0.03 | −0.19 | 0.18 | 0.2725 | AA860184 | PDE8B | CDNA FLJ25435 fis, |
| | | | | | | | | | | | | | clone TST08040 |
| 237172_at | −1.56 | 0.0553 | 2.00 | 22.69 | 14.51 | 0.10 | 0.07 | −0.19 | 0.10 | 0.0590 | AI521891 | na | Transcribed locus |
| 1556655_s_at | −1.56 | 0.0052 | 3.06 | 13.99 | 8.94 | 0.06 | 0.04 | −0.19 | 0.07 | 0.0062 | AI860021 | na | wm22h08.x1 |
| | | | | | | | | | | | | | NCI_CGAP_Ut4 Homo |
| | | | | | | | | | | | | | sapiens cDNA clone |
| | | | | | | | | | | | | | IMAGE: 2436735 3′ |
| | | | | | | | | | | | | | similar to contains Alu |
| | | | | | | | | | | | | | repetitive element; contains |
| | | | | | | | | | | | | | element MER40 repetitive |
| | | | | | | | | | | | | | element; mRNA |
| | | | | | | | | | | | | | sequence. |
| 231887_s_at | −1.57 | 0.1258 | 1.58 | 9.77 | 6.24 | 0.05 | 0.03 | −0.19 | 0.13 | 0.1458 | AB033100 | KIAA1274 | KIAA1274 |
| 244507_at | −1.57 | 0.3407 | 0.97 | 7.88 | 5.03 | 0.04 | 0.02 | −0.19 | 0.19 | 0.2895 | AA905023 | TTC28 | ok09c06.s1 |
| | | | | | | | | | | | | | Soares_NFL_T_GBC_S1 |
| | | | | | | | | | | | | | Homo sapiens cDNA clone |
| | | | | | | | | | | | | | IMAGE: 1507306 3′, |
| | | | | | | | | | | | | | mRNA sequence. |
| 242971_at | −1.57 | 0.0739 | 1.86 | 7.27 | 4.64 | 0.03 | 0.02 | −0.19 | 0.15 | 0.1959 | BF514491 | SH3GLP3 | Transcribed locus |
| 230388_s_at | −1.57 | 0.0562 | 2.00 | 257.07 | 164.12 | 1.18 | 0.76 | −0.19 | 0.10 | 0.0596 | AI797017 | LOC644246 | hypothetical protein |
| | | | | | | | | | | | | | LOC644246 |
| 238248_at | −1.57 | 0.0911 | 1.77 | 6.73 | 4.30 | 0.03 | 0.02 | −0.20 | 0.18 | 0.2881 | AI935789 | UMOD | Uromodulin (uromucoid, |
| | | | | | | | | | | | | | Tamm-Horsfall |
| | | | | | | | | | | | | | glycoprotein) |
| 210254_at | −1.57 | 0.0416 | 2.15 | 329.73 | 210.27 | 1.52 | 0.98 | −0.20 | 0.10 | 0.0435 | L35848 | MS4A3 | Membrane-spanning 4- |
| | | | | | | | | | | | | | domains, subfamily A, |
| | | | | | | | | | | | | | member 3 (hematopoietic |
| | | | | | | | | | | | | | cell-specific) |
| 237507_at | −1.57 | 0.5648 | 0.58 | 18.96 | 12.08 | 0.09 | 0.06 | −0.20 | 0.20 | 0.3247 | AI333069 | KRT73 | Keratin 6 irs3 |
| 1565879_at | −1.57 | 0.6704 | 0.43 | 6.95 | 4.43 | 0.03 | 0.02 | −0.20 | 0.22 | 0.3704 | R99095 | SLC5A11 | Solute carrier family 5 |
| | | | | | | | | | | | | | (sodium/glucose |
| | | | | | | | | | | | | | cotransporter), member 11 |
| 212793_at | −1.57 | 0.1655 | 1.42 | 50.75 | 32.31 | 0.23 | 0.15 | −0.20 | 0.18 | 0.2722 | BF513244 | DAAM2 | Dishevelled associated |
| | | | | | | | | | | | | | activator of morphogenesis 2 |
| 212805_at | −1.57 | 0.4498 | 0.77 | 61.00 | 38.83 | 0.28 | 0.18 | −0.20 | 0.18 | 0.2594 | AB002365 | KIAA0367 | KIAA0367 |
| 217036_at | −1.57 | 0.1613 | 1.45 | 128.50 | 81.79 | 0.59 | 0.38 | −0.20 | 0.12 | 0.1061 | AF103530 | 217036_at | Immunoglobulin kappa |
| | | | | | | | | | | | | | chain, V-region (SPK.3) |
| 233554_at | −1.57 | 0.1749 | 1.39 | 99.68 | 63.37 | 0.46 | 0.29 | −0.20 | 0.11 | 0.0598 | AF339764 | PHGDHL1 | Phosphoglycerate |
| | | | | | | | | | | | | | dehydrogenase like 1 |
| 208136_s_at | −1.57 | 0.0181 | 2.51 | 12.50 | 7.94 | 0.06 | 0.04 | −0.20 | 0.10 | 0.0456 | NM_030970 | MGC3771 | hypothetical protein |
| | | | | | | | | | | | | | MGC3771 |
| 226471_at | −1.58 | 0.0365 | 2.20 | 6.76 | 4.29 | 0.03 | 0.02 | −0.20 | 0.12 | 0.0949 | AI423493 | GGTL3 | Gamma- |
| | | | | | | | | | | | | | glutamyltransferase-like 3 |
| 205379_at | −1.58 | 0.2316 | 1.22 | 36.02 | 22.78 | 0.17 | 0.11 | −0.20 | 0.15 | 0.1765 | NM_001236 | CBR3 | Carbonyl reductase 3 |
| 243038_at | −1.58 | 0.1031 | 1.70 | 7.02 | 4.43 | 0.03 | 0.02 | −0.20 | 0.15 | 0.1954 | AW292769 | C2orf38 | Chromosome 2 open |
| | | | | | | | | | | | | | reading frame 38 |
| 215103_at | −1.58 | 0.0800 | 1.82 | 7.55 | 4.77 | 0.03 | 0.02 | −0.20 | 0.13 | 0.0999 | AW192911 | CYP2C18 | Cytochrome P450, family |
| | | | | | | | | | | | | | 2, subfamily C, |
| | | | | | | | | | | | | | polypeptide 18 |
| 244421_at | −1.59 | 0.1494 | 1.48 | 9.95 | 6.27 | 0.05 | 0.03 | −0.20 | 0.12 | 0.0944 | BF434110 | MLF2 | Myeloid leukemia factor 2 |
| 233020_at | −1.59 | 0.0113 | 2.71 | 43.40 | 27.33 | 0.20 | 0.13 | −0.20 | 0.09 | 0.0253 | AU154125 | SEC22B | SEC22 vesicle trafficking |
| | | | | | | | | | | | | | protein homolog B (S. cerevisiae) |
| 1562307_at | −1.59 | 0.1308 | 1.56 | 78.79 | 49.59 | 0.36 | 0.23 | −0.20 | 0.10 | 0.0483 | AL832657 | RNF24 | Ring finger protein 24 |
| 205438_at | −1.59 | 0.1074 | 1.67 | 11.11 | 6.98 | 0.05 | 0.03 | −0.20 | 0.16 | 0.2118 | NM_007039 | PTPN21 | Protein tyrosine |
| | | | | | | | | | | | | | phosphatase, non-receptor |
| | | | | | | | | | | | | | type 21 |
| 239359_at | −1.59 | 0.0686 | 1.89 | 7.02 | 4.41 | 0.03 | 0.02 | −0.20 | 0.13 | 0.1230 | AA383208 | LOC441061 | similar to membrane- |
| | | | | | | | | | | | | | associated ring finger |
| | | | | | | | | | | | | | (C3HC4) 4 |
| 221930_at | −1.59 | 0.0287 | 2.31 | 10.66 | 6.70 | 0.05 | 0.03 | −0.20 | 0.19 | 0.2755 | AI217472 | PHF7 | PHD finger protein 7 |
| 220234_at | −1.59 | 0.0030 | 3.27 | 14.73 | 9.26 | 0.07 | 0.04 | −0.20 | 0.08 | 0.0088 | NM_004056 | CA8 | Carbonic anhydrase VIII |
| 202018_s_at | −1.59 | 0.0560 | 2.00 | 1823.30 | 1145.37 | 8.40 | 5.32 | −0.20 | 0.11 | 0.0682 | NM_002343 | LTF | Lactotransferrin |
| 1559814_at | −1.59 | 0.0183 | 2.51 | 6.67 | 4.18 | 0.03 | 0.02 | −0.20 | 0.11 | 0.0633 | AK024712 | CSS3 | chondroitin sulfate |
| | | | | | | | | | | | | | synthase 3 |
| 211364_at | −1.59 | 0.0034 | 3.25 | 7.09 | 4.45 | 0.03 | 0.02 | −0.20 | 0.08 | 0.0095 | AF109294 | MTAP | Methylthioadenosine |
| | | | | | | | | | | | | | phosphorylase |
| 244045_at | −1.60 | 0.1546 | 1.48 | 7.90 | 4.95 | 0.04 | 0.02 | −0.20 | 0.14 | 0.1479 | N66930 | 244045_at | za47h10.s1 Soares fetal |
| | | | | | | | | | | | | | liver spleen 1NFLS Homo |
| | | | | | | | | | | | | | sapiens cDNA clone |
| | | | | | | | | | | | | | IMAGE: 295747 3′ similar |
| | | | | | | | | | | | | | to contains Alu repetitive |
| | | | | | | | | | | | | | element; mRNA sequence. |
| 212187_x_at | −1.60 | 0.0127 | 2.69 | 93.59 | 58.62 | 0.43 | 0.27 | −0.20 | 0.11 | 0.0747 | NM_000954 | PTGDS | Prostaglandin D2 synthase |
| | | | | | | | | | | | | | 21 kDa (brain) |
| 205040_at | −1.60 | 0.0844 | 1.80 | 557.11 | 348.93 | 2.57 | 1.62 | −0.20 | 0.13 | 0.1206 | NM_000607 | ORM1 | Orosomucoid 1 |
| 242267_x_at | −1.60 | 0.2285 | 1.24 | 93.79 | 58.74 | 0.43 | 0.27 | −0.20 | 0.18 | 0.2480 | T68304 | NINJ2 | Ninjurin 2 |
| 1565617_at | −1.60 | 0.0029 | 3.34 | 9.94 | 6.23 | 0.05 | 0.03 | −0.20 | 0.08 | 0.0132 | AK097628 | STMN3 | CDNA FLJ40309 fis, |
| | | | | | | | | | | | | | clone TESTI2029470 |
| 209975_at | −1.60 | 0.0424 | 2.13 | 18.83 | 11.79 | 0.09 | 0.05 | −0.20 | 0.14 | 0.1379 | AF182276 | CYP2E1 | Cytochrome P450, family |
| | | | | | | | | | | | | | 2, subfamily E, |
| | | | | | | | | | | | | | polypeptide 1 |
| 221521_s_at | −1.60 | 0.0216 | 2.46 | 38.97 | 24.40 | 0.18 | 0.11 | −0.20 | 0.09 | 0.0320 | BC003186 | GINS2 | GINS complex subunit 2 |
| | | | | | | | | | | | | | (Psf2 homolog) |
| 212768_s_at | −1.60 | 0.2892 | 1.08 | 530.05 | 331.84 | 2.44 | 1.54 | −0.20 | 0.18 | 0.2475 | AL390736 | OLFM4 | Human DNA sequence |
| | | | | | | | | | | | | | from clone RP11-209J19 |
| | | | | | | | | | | | | | on chromosome 13 |
| | | | | | | | | | | | | | Contains a novel gene, |
| | | | | | | | | | | | | | complete sequence. |
| 1553972_a_at | −1.60 | 0.0142 | 2.66 | 77.51 | 48.51 | 0.36 | 0.23 | −0.20 | 0.11 | 0.0645 | BC007257 | CBS | Cystathionine-beta- |
| | | | | | | | | | | | | | synthase |
| 231032_at | −1.60 | 0.3004 | 1.06 | 7.10 | 4.44 | 0.03 | 0.02 | −0.20 | 0.15 | 0.1847 | BE503158 | LOC286071 | hypothetical protein |
| | | | | | | | | | | | | | LOC286071 |
| 208211_s_at | −1.60 | 0.0076 | 2.88 | 7.92 | 4.94 | 0.04 | 0.02 | −0.21 | 0.10 | 0.0455 | U66559 | ALK | Anaplastic lymphoma |
| | | | | | | | | | | | | | kinase (Ki-1) |
| 231067_s_at | −1.60 | 0.0324 | 2.26 | 42.17 | 26.29 | 0.19 | 0.12 | −0.21 | 0.09 | 0.0166 | BF114967 | AKAP12 | A kinase (PRKA) anchor |
| | | | | | | | | | | | | | protein (gravin) 12 |
| 209773_s_at | −1.61 | 0.0338 | 2.26 | 127.52 | 79.44 | 0.59 | 0.37 | −0.21 | 0.11 | 0.0667 | BC001886 | RRM2 | Ribonucleotide reductase |
| | | | | | | | | | | | | | M2 polypeptide |
| 202095_s_at | −1.61 | 0.1911 | 1.36 | 36.19 | 22.53 | 0.17 | 0.10 | −0.21 | 0.21 | 0.3394 | NM_001168 | BIRC5 | Baculoviral IAP repeat- |
| | | | | | | | | | | | | | containing 5 (survivin) |
| 209693_at | −1.61 | 0.5274 | 0.64 | 14.71 | 9.15 | 0.07 | 0.04 | −0.21 | 0.27 | 0.4445 | AF116574 | ASTN2 | Astrotactin 2 |
| 244506_at | −1.61 | 0.0182 | 2.51 | 9.13 | 5.68 | 0.04 | 0.03 | −0.21 | 0.10 | 0.0438 | BE297946 | TMTC1 | Transmembrane and |
| | | | | | | | | | | | | | tetratricopeptide repeat |
| | | | | | | | | | | | | | containing 1 |
| 219629_at | −1.61 | 0.4896 | 0.70 | 87.52 | 54.43 | 0.40 | 0.25 | −0.21 | 0.27 | 0.4350 | NM_017911 | FAM118A | Family with sequence |
| | | | | | | | | | | | | | similarity 118, member A |
| 216643_at | −1.61 | 0.2838 | 1.10 | 6.90 | 4.28 | 0.03 | 0.02 | −0.21 | 0.22 | 0.3514 | D25272 | KCNIP4 | Kv channel interacting |
| | | | | | | | | | | | | | protein 4 |
| 1559732_at | −1.61 | 0.0032 | 3.23 | 10.87 | 6.74 | 0.05 | 0.03 | −0.21 | 0.09 | 0.0208 | AK056624 | KCNH2 | Potassium voltage-gated |
| | | | | | | | | | | | | | channel, subfamily H (eag- |
| | | | | | | | | | | | | | related), member 2 |
| 231236_at | −1.61 | 0.1026 | 1.69 | 25.42 | 15.77 | 0.12 | 0.07 | −0.21 | 0.31 | 0.4908 | AW440310 | ZFP57 | Zinc finger protein 57 |
| | | | | | | | | | | | | | homolog (mouse) |
| 237432_at | −1.61 | 0.3533 | 0.95 | 8.18 | 5.07 | 0.04 | 0.02 | −0.21 | 0.23 | 0.3578 | BF514363 | LOC401911 | similar to 60S ribosomal |
| | | | | | | | | | | | | | protein L29 (Cell surface |
| | | | | | | | | | | | | | heparin binding protein |
| | | | | | | | | | | | | | HIP) |
| 236646_at | −1.61 | 0.1460 | 1.50 | 12.21 | 7.56 | 0.06 | 0.04 | −0.21 | 0.16 | 0.1808 | BE301029 | C12orf59 | Chromosome 12 open |
| | | | | | | | | | | | | | reading frame 59 |
| 1555896_a_at | −1.62 | 0.1094 | 1.65 | 8.09 | 5.01 | 0.04 | 0.02 | −0.21 | 0.20 | 0.2950 | BM973999 | ADAM15 | ADAM metallopeptidase |
| | | | | | | | | | | | | | domain 15 (metargidin) |
| 226523_at | −1.62 | 0.0040 | 3.15 | 9.02 | 5.58 | 0.04 | 0.03 | −0.21 | 0.11 | 0.0444 | AI082237 | TAGLN | Transgelin |
| 229839_at | −1.62 | 0.2447 | 1.19 | 8.09 | 5.01 | 0.04 | 0.02 | −0.21 | 0.20 | 0.2998 | AI799784 | SCARA5 | Scavenger receptor class |
| | | | | | | | | | | | | | A, member 5 (putative) |
| 208470_s_at | −1.62 | 0.0206 | 2.46 | 288.73 | 178.57 | 1.33 | 0.83 | −0.21 | 0.09 | 0.0151 | NM_020995 | HPR | Haptoglobin |
| 208515_at | −1.62 | 0.0536 | 2.02 | 6.77 | 4.19 | 0.03 | 0.02 | −0.21 | 0.12 | 0.0773 | NM_003521 | HIST1H2BM | Histone 1, H2bm |
| 234531_at | −1.62 | 0.0599 | 1.96 | 9.17 | 5.67 | 0.04 | 0.03 | −0.21 | 0.16 | 0.1751 | AK023440 | FLJ11292 | CDNA FLJ13378 fis, |
| | | | | | | | | | | | | | clone PLACE1000931 |
| 228696_at | −1.62 | 0.0155 | 2.61 | 39.14 | 24.19 | 0.18 | 0.11 | −0.21 | 0.09 | 0.0205 | AA631143 | SLC45A3 | Solute carrier family 45, |
| | | | | | | | | | | | | | member 3 |
| 244875_at | −1.62 | 0.0960 | 1.73 | 9.73 | 6.01 | 0.04 | 0.03 | −0.21 | 0.15 | 0.1542 | AA548751 | CXYorf2 | Chromosome X and Y |
| | | | | | | | | | | | | | open reading frame 2 |
| 239628_at | −1.62 | 0.0864 | 1.78 | 6.91 | 4.26 | 0.03 | 0.02 | −0.21 | 0.15 | 0.1509 | AI565624 | 239628_at | Transcribed locus, |
| | | | | | | | | | | | | | moderately similar to |
| | | | | | | | | | | | | | XP_515629.1 |
| | | | | | | | | | | | | | PREDICTED: similar to |
| | | | | | | | | | | | | | U5 snRNP-specific |
| | | | | | | | | | | | | | protein, 200 kDa; U5 |
| | | | | | | | | | | | | | snRNP-specific protein, |
| | | | | | | | | | | | | | 200 kDa (DEXH RNA |
| | | | | | | | | | | | | | helicase family) [Pantroglodytes] |
| 217438_at | −1.62 | 0.5303 | 0.64 | 7.72 | 4.76 | 0.04 | 0.02 | −0.21 | 0.21 | 0.3306 | D25272 | KCNIP4 | Kv channel interacting |
| | | | | | | | | | | | | | protein 4 |
| 233840_at | −1.62 | 0.0933 | 1.74 | 14.45 | 8.90 | 0.07 | 0.04 | −0.21 | 0.12 | 0.0691 | AK021878 | LOC284017 | hypothetical protein |
| | | | | | | | | | | | | | LOC284017 |
| 205531_s_at | −1.62 | 0.1723 | 1.40 | 15.22 | 9.37 | 0.07 | 0.04 | −0.21 | 0.15 | 0.1573 | NM_013267 | GLS2 | Glutaminase 2 (liver, |
| | | | | | | | | | | | | | mitochondrial) |
| 231107_at | −1.62 | 0.1503 | 1.48 | 31.72 | 19.53 | 0.15 | 0.09 | −0.21 | 0.12 | 0.0825 | AI492822 | RSU1 | Ras suppressor protein 1 |
| 1563745_a_at | −1.63 | 0.0496 | 2.06 | 7.12 | 4.38 | 0.03 | 0.02 | −0.21 | 0.10 | 0.0261 | AK098249 | LOC283050 | hypothetical protein |
| | | | | | | | | | | | | | LOC283050 |
| 1559003_a_at | −1.63 | 0.0526 | 2.03 | 207.36 | 127.46 | 0.96 | 0.59 | −0.21 | 0.12 | 0.0893 | AK054714 | LOC126661 | hypothetical protein |
| | | | | | | | | | | | | | LOC126661 |
| 1556131_s_at | −1.63 | 0.0831 | 1.80 | 9.12 | 5.60 | 0.04 | 0.03 | −0.21 | 0.12 | 0.0670 | AK074045 | FBF1 | Fas (TNFRSF6) binding |
| | | | | | | | | | | | | | factor 1 |
| 234350_at | −1.63 | 0.0735 | 1.87 | 10.02 | 6.15 | 0.05 | 0.03 | −0.21 | 0.13 | 0.0934 | AF127125 | IGL | Homo sapiens isolate 459 |
| | | | | | | | | | | | | | immunoglobulin lambda |
| | | | | | | | | | | | | | light chain variable region |
| | | | | | | | | | | | | | (IGL) gene, partial cds. |
| 1564383_s_at | −1.63 | 0.1957 | 1.33 | 23.71 | 14.55 | 0.11 | 0.07 | −0.21 | 0.10 | 0.0401 | AK093253 | FLJ35934 | FLJ35934 protein |
| 204916_at | −1.63 | 0.0332 | 2.24 | 10.30 | 6.31 | 0.05 | 0.03 | −0.21 | 0.12 | 0.0697 | NM_005855 | RAMP1 | Receptor (calcitonin) |
| | | | | | | | | | | | | | activity modifying protein 1 |
| 205056_s_at | −1.63 | 0.0332 | 2.24 | 33.55 | 20.56 | 0.15 | 0.10 | −0.21 | 0.11 | 0.0412 | NM_019858 | GPR162 | G protein-coupled receptor |
| | | | | | | | | | | | | | 162 |
| 239754_at | −1.63 | 0.0772 | 1.84 | 13.13 | 8.04 | 0.06 | 0.04 | −0.21 | 0.23 | 0.3435 | BE671886 | C17orf45 | Chromosome 17 open |
| | | | | | | | | | | | | | reading frame 45 |
| 224458_at | −1.64 | 0.0681 | 1.90 | 8.10 | 4.95 | 0.04 | 0.02 | −0.21 | 0.11 | 0.0487 | BC006115 | C9orf125 | Chromosome 9 open |
| | | | | | | | | | | | | | reading frame 125 |
| 204347_at | −1.64 | 0.0062 | 3.03 | 8.02 | 4.90 | 0.04 | 0.02 | −0.21 | 0.11 | 0.0416 | AI653169 | AK3L1 | Transcribed locus |
| 211877_s_at | −1.64 | 0.0425 | 2.13 | 9.16 | 5.60 | 0.04 | 0.03 | −0.21 | 0.15 | 0.1372 | AF152505 | PCDHGA11 | Protocadherin gamma |
| | | | | | | | | | | | | | subfamily C, 3 |
| 1562446_at | −1.64 | 0.2845 | 1.10 | 8.33 | 5.09 | 0.04 | 0.02 | −0.21 | 0.23 | 0.3674 | BG722372 | LOC441136 | hypothetical gene |
| | | | | | | | | | | | | | supported by AK092633 |
| 216215_s_at | −1.64 | 0.0131 | 2.65 | 13.50 | 8.24 | 0.06 | 0.04 | −0.21 | 0.10 | 0.0269 | AL049748 | RBM9 | Human DNA sequence |
| | | | | | | | | | | | | | from clone RP1-41P2 on |
| | | | | | | | | | | | | | chromosome 22, complete |
| | | | | | | | | | | | | | sequence. |
| 222293_at | −1.64 | 0.0267 | 2.34 | 9.83 | 5.99 | 0.05 | 0.03 | −0.21 | 0.15 | 0.1313 | AW204383 | IGSF4C | Immunoglobulin |
| | | | | | | | | | | | | | superfamily, member 4C |
| 206560_s_at | −1.64 | 0.7278 | 0.35 | 20.99 | 12.80 | 0.10 | 0.06 | −0.22 | 0.12 | 0.0706 | NM_006533 | MIA | RAB4B, member RAS |
| | | | | | | | | | | | | | oncogene family |
| 230776_at | −1.64 | 0.1984 | 1.32 | 26.15 | 15.93 | 0.12 | 0.07 | −0.22 | 0.16 | 0.1869 | N59856 | RNF157 | Ring finger protein 157 |
| 209973_at | −1.64 | 0.3316 | 0.99 | 8.15 | 4.97 | 0.04 | 0.02 | −0.22 | 0.22 | 0.3259 | AF097419 | NFKBIL1 | Nuclear factor of kappa |
| | | | | | | | | | | | | | light polypeptide gene |
| | | | | | | | | | | | | | enhancer in B-cells |
| | | | | | | | | | | | | | inhibitor-like 1 |
| 232806_s_at | −1.64 | 0.0591 | 1.98 | 13.96 | 8.50 | 0.06 | 0.04 | −0.22 | 0.11 | 0.0333 | AU158601 | C3orf40 | Chromosome 3 open |
| | | | | | | | | | | | | | reading frame 40 |
| 214973_x_at | −1.64 | 0.6442 | 0.47 | 53.34 | 32.49 | 0.25 | 0.15 | −0.22 | 0.27 | 0.4341 | AJ275469 | IGVH3 | Homo sapiens partial |
| | | | | | | | | | | | | | IGVH3 gene for |
| | | | | | | | | | | | | | immunoglobulin heavy |
| | | | | | | | | | | | | | chain V region, case 2, cell |
| | | | | | | | | | | | | | E 172. |
| 214533_at | −1.64 | 0.1557 | 1.48 | 6.88 | 4.19 | 0.03 | 0.02 | −0.22 | 0.15 | 0.1258 | NM_001836 | CMA1 | Chymase 1, mast cell |
| 1560480_at | −1.65 | 0.6317 | 0.48 | 17.48 | 10.61 | 0.08 | 0.05 | −0.22 | 0.15 | 0.1388 | BC036927 | PLGLB1 | Homo sapiens, clone |
| | | | | | | | | | | | | | IMAGE: 5223521, mRNA |
| 1554288_at | −1.65 | 0.2122 | 1.28 | 14.67 | 8.86 | 0.07 | 0.04 | −0.22 | 0.19 | 0.2469 | BC037207 | KIAA1600 | KIAA1600 |
| 231305_at | −1.66 | 0.0053 | 3.02 | 14.51 | 8.75 | 0.07 | 0.04 | −0.22 | 0.08 | 0.0052 | AI820801 | LOC643982 | hypothetical protein |
| | | | | | | | | | | | | | LOC643982 |
| 239530_at | −1.66 | 0.0065 | 2.94 | 10.93 | 6.58 | 0.05 | 0.03 | −0.22 | 0.10 | 0.0257 | BG171323 | ADD2 | Clone 23700 mRNA |
| | | | | | | | | | | | | | sequence |
| 1567706_at | −1.66 | 0.0642 | 1.95 | 6.71 | 4.04 | 0.03 | 0.02 | −0.22 | 0.13 | 0.0845 | AF009316 | SEMA5A | Sema domain, seven |
| | | | | | | | | | | | | | thrombospondin repeats |
| | | | | | | | | | | | | | (type 1 and type 1-like), |
| | | | | | | | | | | | | | transmembrane domain |
| | | | | | | | | | | | | | (TM) and short |
| | | | | | | | | | | | | | cytoplasmic domain, |
| | | | | | | | | | | | | | (semaphorin) 5A |
| 218815_s_at | −1.66 | 0.0368 | 2.20 | 25.45 | 15.29 | 0.12 | 0.07 | −0.22 | 0.12 | 0.0580 | NM_018022 | TMEM51 | Transmembrane protein 51 |
| 205899_at | −1.67 | 0.1091 | 1.65 | 9.37 | 5.63 | 0.04 | 0.03 | −0.22 | 0.23 | 0.3279 | NM_003914 | CCNA1 | Cyclin A1 |
| 207067_s_at | −1.67 | 0.0276 | 2.33 | 103.65 | 62.04 | 0.48 | 0.29 | −0.22 | 0.11 | 0.0341 | NM_002112 | HDC | Histidine decarboxylase |
| 215709_at | −1.67 | 0.1909 | 1.35 | 6.76 | 4.04 | 0.03 | 0.02 | −0.22 | 0.15 | 0.1138 | AL121975 | PRIM2A | Human DNA sequence |
| | | | | | | | | | | | | | from clone RP3-422B11 |
| | | | | | | | | | | | | | on chromosome 6p11.2-12.3 |
| | | | | | | | | | | | | | Contains part of the |
| | | | | | | | | | | | | | PRIM2A gene for primase |
| | | | | | | | | | | | | | polypeptide 2A (58 kD), |
| | | | | | | | | | | | | | complete sequence. |
| 206893_at | −1.67 | 0.0489 | 2.09 | 8.30 | 4.97 | 0.04 | 0.02 | −0.22 | 0.16 | 0.1694 | NM_002968 | SALL1 | Sal-like 1 (Drosophila) |
| 228054_at | −1.67 | 0.1562 | 1.47 | 13.41 | 8.01 | 0.06 | 0.04 | −0.22 | 0.19 | 0.2447 | BF593660 | TMEM44 | Transmembrane protein 44 |
| 207950_s_at | −1.67 | 0.0060 | 2.99 | 16.37 | 9.77 | 0.08 | 0.05 | −0.22 | 0.10 | 0.0181 | NM_001149 | ANK3 | Ankyrin 3, node of |
| | | | | | | | | | | | | | Ranvier (ankyrin G) |
| 220344_at | −1.68 | 0.0579 | 1.99 | 7.58 | 4.52 | 0.03 | 0.02 | −0.22 | 0.19 | 0.2278 | NM_020643 | C11orf16 | Chromosome 11 open |
| | | | | | | | | | | | | | reading frame 16 |
| 210497_x_at | −1.68 | 0.2956 | 1.07 | 6.62 | 3.95 | 0.03 | 0.02 | −0.22 | 0.20 | 0.2584 | BC002818 | SSX2 | Synovial sarcoma, X |
| | | | | | | | | | | | | | breakpoint 2 |
| 231285_at | −1.68 | 0.1026 | 1.69 | 6.72 | 4.00 | 0.03 | 0.02 | −0.22 | 0.13 | 0.0724 | AI458583 | STT3B | STT3, subunit of the |
| | | | | | | | | | | | | | oligosaccharyltransferase |
| | | | | | | | | | | | | | complex, homolog B (S. cerevisiae) |
| 209764_at | −1.68 | 0.0597 | 1.97 | 17.08 | 10.16 | 0.08 | 0.05 | −0.23 | 0.12 | 0.0619 | AL022312 | MGAT3 | Human DNA sequence |
| | | | | | | | | | | | | | from clone RP5-1104E15 |
| | | | | | | | | | | | | | on chromosome 22q12.3-13.1 |
| | | | | | | | | | | | | | Contains the MGAT3 |
| | | | | | | | | | | | | | gene for mannosyl (beta- |
| | | | | | | | | | | | | | 1,4-)-glycoprotein beta- |
| | | | | | | | | | | | | | 1,4-N- |
| | | | | | | | | | | | | | acetylglucosaminyltransferase, |
| | | | | | | | | | | | | | the gene for a |
| | | | | | | | | | | | | | predicted protein, the |
| | | | | | | | | | | | | | ATF4 gene for activating |
| | | | | | | | | | | | | | transcription factor 4 (tax- |
| | | | | | | | | | | | | | responsive enhancer |
| | | | | | | | | | | | | | element B67) and the 5′ |
| | | | | | | | | | | | | | end of the CACNA1I gene |
| | | | | | | | | | | | | | for voltage-dependent |
| | | | | | | | | | | | | | calcium channel, alpha 1I |
| | | | | | | | | | | | | | subunit. Contains ESTs, |
| | | | | | | | | | | | | | STSs, GSSs and five |
| | | | | | | | | | | | | | putative CpG islands, |
| | | | | | | | | | | | | | complete sequence. |
| 229370_at | −1.68 | 0.1305 | 1.56 | 8.16 | 4.85 | 0.04 | 0.02 | −0.23 | 0.18 | 0.1991 | BF507344 | LOC441303 | Transcribed locus, strongly |
| | | | | | | | | | | | | | similar to XP_499090.1 |
| | | | | | | | | | | | | | PREDICTED: hypothetical |
| | | | | | | | | | | | | | protein XP_499090 [Homo |
| | | | | | | | | | | | | | sapiens] |
| 203290_at | −1.68 | 0.3713 | 0.91 | 725.73 | 430.74 | 3.34 | 2.00 | −0.23 | 0.27 | 0.4016 | NM_002122 | HLA-DQA1 | Major histocompatibility |
| | | | | | | | | | | | | | complex, class II, DQ |
| | | | | | | | | | | | | | alpha 1 |
| 211430_s_at | −1.69 | 0.0409 | 2.14 | 570.89 | 338.16 | 2.63 | 1.57 | −0.23 | 0.12 | 0.0597 | M87789 | IGHG3 | Immunoglobulin heavy |
| | | | | | | | | | | | | | constant gamma 1 (G1m |
| | | | | | | | | | | | | | marker) |
| 1556805_at | −1.69 | 0.1005 | 1.71 | 10.29 | 6.09 | 0.05 | 0.03 | −0.23 | 0.13 | 0.0637 | BC042834 | 1556805_at | CDNA clone |
| | | | | | | | | | | | | | IMAGE: 5314388 |
| 228868_x_at | −1.69 | 0.1711 | 1.41 | 21.18 | 12.51 | 0.10 | 0.06 | −0.23 | 0.19 | 0.2282 | AW075105 | CDT1 | Chromatin licensing and |
| | | | | | | | | | | | | | DNA replication factor 1 |
| 224128_at | −1.70 | 0.0357 | 2.21 | 10.07 | 5.94 | 0.05 | 0.03 | −0.23 | 0.10 | 0.0190 | BC002769 | C20orf43 | Chromosome 20 open |
| | | | | | | | | | | | | | reading frame 43 |
| 237203_at | −1.70 | 0.2860 | 1.09 | 14.94 | 8.81 | 0.07 | 0.04 | −0.23 | 0.21 | 0.2565 | BE466578 | 237203_at | Homo sapiens, clone |
| | | | | | | | | | | | | | IMAGE: 4214313, mRNA |
| 239402_at | −1.70 | 0.0717 | 1.89 | 6.75 | 3.97 | 0.03 | 0.02 | −0.23 | 0.16 | 0.1444 | AW117206 | COX10 | Transcribed locus |
| 212816_s_at | −1.70 | 0.0353 | 2.26 | 48.29 | 28.38 | 0.22 | 0.13 | −0.23 | 0.16 | 0.1194 | BE613178 | CBS | Cystathionine-beta- |
| | | | | | | | | | | | | | synthase |
| 235928_at | −1.70 | 0.0957 | 1.73 | 9.31 | 5.47 | 0.04 | 0.03 | −0.23 | 0.15 | 0.1161 | BF114894 | C10orf41 | Transcribed locus |
| 244524_at | −1.70 | 0.2396 | 1.20 | 49.35 | 28.98 | 0.23 | 0.13 | −0.23 | 0.24 | 0.3248 | AI587332 | PAX8 | Paired box gene 8 |
| 227457_at | −1.70 | 0.0805 | 1.83 | 12.65 | 7.43 | 0.06 | 0.03 | −0.23 | 0.20 | 0.2416 | AB046843 | TMEM16H | Transmembrane protein |
| | | | | | | | | | | | | | 16H |
| 1563346_at | −1.71 | 0.0650 | 1.96 | 8.29 | 4.86 | 0.04 | 0.02 | −0.23 | 0.16 | 0.1379 | AY063452 | D21S2089E | D21S2089E |
| 213520_at | −1.71 | 0.0016 | 3.50 | 25.77 | 15.09 | 0.12 | 0.07 | −0.23 | 0.07 | 0.0003 | NM_004260 | RECQL4 | RecQ protein-like 4 |
| 1561616_a_at | −1.71 | 0.1627 | 1.46 | 6.77 | 3.96 | 0.03 | 0.02 | −0.23 | 0.17 | 0.1820 | BU616806 | DNAH6 | Dynein, axonemal, heavy |
| | | | | | | | | | | | | | polypeptide 6 |
| 91684_g_at | −1.71 | 0.0061 | 3.04 | 23.03 | 13.47 | 0.11 | 0.06 | −0.23 | 0.11 | 0.0251 | AI571298 | EXOSC4 | Exosome component 4 |
| 231598_x_at | −1.71 | 0.0721 | 1.87 | 9.79 | 5.73 | 0.05 | 0.03 | −0.23 | 0.11 | 0.0255 | AI379823 | 231598_x_at | tb91d12.x1 |
| | | | | | | | | | | | | | NCI_CGAP_Lu25 Homo |
| | | | | | | | | | | | | | sapiens cDNA clone |
| | | | | | | | | | | | | | IMAGE: 2061719 3′, |
| | | | | | | | | | | | | | mRNA sequence. |
| 201292_at | −1.71 | 0.0116 | 2.79 | 49.85 | 29.14 | 0.23 | 0.14 | −0.23 | 0.11 | 0.0364 | AL561834 | TOP2A | Topoisomerase (DNA) II |
| | | | | | | | | | | | | | alpha 170 kDa |
| 205624_at | −1.71 | 0.0016 | 3.50 | 175.65 | 102.53 | 0.81 | 0.48 | −0.23 | 0.08 | 0.0019 | NM_001870 | CPA3 | Carboxypeptidase A3 |
| | | | | | | | | | | | | | (mast cell) |
| 232121_at | −1.72 | 0.1417 | 1.51 | 11.44 | 6.65 | 0.05 | 0.03 | −0.24 | 0.14 | 0.0796 | AK021659 | DNMT2 | DNA (cytosine-5-)- |
| | | | | | | | | | | | | | methyltransferase 2 |
| 209710_at | −1.72 | 0.0027 | 3.34 | 184.75 | 107.39 | 0.85 | 0.50 | −0.24 | 0.09 | 0.0057 | AL563460 | GATA2 | GATA binding protein 2 |
| 207329_at | −1.73 | 0.1427 | 1.51 | 66.99 | 38.79 | 0.31 | 0.18 | −0.24 | 0.17 | 0.1552 | NM_002424 | MMP8 | Matrix metallopeptidase 8 |
| | | | | | | | | | | | | | (neutrophil collagenase) |
| 220051_at | −1.73 | 0.0710 | 1.89 | 19.25 | 11.14 | 0.09 | 0.05 | −0.24 | 0.14 | 0.0879 | NM_006799 | PRSS21 | Protease, serine, 21 |
| | | | | | | | | | | | | | (testisin) |
| 1554396_at | −1.73 | 0.1512 | 1.48 | 10.03 | 5.80 | 0.05 | 0.03 | −0.24 | 0.14 | 0.0866 | BC011011 | UEVLD | Ubiquitin-conjugating |
| | | | | | | | | | | | | | enzyme E2-like |
| 241679_at | −1.73 | 0.0310 | 2.27 | 8.17 | 4.71 | 0.04 | 0.02 | −0.24 | 0.12 | 0.0454 | AI672553 | AKAP12 | A kinase (PRKA) anchor |
| | | | | | | | | | | | | | protein (gravin) 12 |
| 205249_at | −1.73 | 0.1920 | 1.34 | 9.09 | 5.24 | 0.04 | 0.02 | −0.24 | 0.14 | 0.0914 | NM_000399 | EGR2 | Early growth response 2 |
| | | | | | | | | | | | | | (Krox-20 homolog, |
| | | | | | | | | | | | | | Drosophila) |
| 1569386_at | −1.74 | 0.0081 | 2.85 | 7.15 | 4.12 | 0.03 | 0.02 | −0.24 | 0.12 | 0.0382 | BC028053 | LOC645677 | similar to ciliary rootlet |
| | | | | | | | | | | | | | coiled-coil, rootletin |
| 1553604_at | −1.74 | 0.0692 | 1.90 | 28.65 | 16.47 | 0.13 | 0.08 | −0.24 | 0.15 | 0.1156 | NM_152701 | ABCA13 | ATP-binding cassette, sub- |
| | | | | | | | | | | | | | family A (ABC1), member |
| | | | | | | | | | | | | | 13 |
| 240663_at | −1.75 | 0.1475 | 1.52 | 7.51 | 4.30 | 0.03 | 0.02 | −0.24 | 0.28 | 0.3825 | AI990613 | WDR41 | WD repeat domain 41 |
| 1552449_a_at | −1.75 | 0.2349 | 1.21 | 25.37 | 14.47 | 0.12 | 0.07 | −0.24 | 0.20 | 0.2096 | NM_145651 | SCGB1C1 | Secretoglobin, family 1C, |
| | | | | | | | | | | | | | member 1 |
| 216426_at | −1.76 | 0.2081 | 1.31 | 14.12 | 8.04 | 0.07 | 0.04 | −0.24 | 0.23 | 0.2908 | AL136318 | LOC644540 | Human DNA sequence |
| | | | | | | | | | | | | | from clone RP1-254P11 on |
| | | | | | | | | | | | | | chromosome 10 Contains a |
| | | | | | | | | | | | | | transcription elongation |
| | | | | | | | | | | | | | factor B (SIII) polypeptide |
| | | | | | | | | | | | | | 1 (15 kDa, elongin C) |
| | | | | | | | | | | | | | (TCEB1) pseudogene, |
| | | | | | | | | | | | | | complete sequence. |
| 212531_at | −1.76 | 0.0833 | 1.80 | 1332.96 | 758.24 | 6.14 | 3.52 | −0.25 | 0.17 | 0.1361 | NM_005564 | LCN2 | Lipocalin 2 (oncogene |
| | | | | | | | | | | | | | 24p3) |
| 1564435_a_at | −1.76 | 0.1866 | 1.35 | 34.80 | 19.78 | 0.16 | 0.09 | −0.25 | 0.20 | 0.2210 | AK093060 | KRT72 | Keratin 5 (epidermolysis |
| | | | | | | | | | | | | | bullosa simplex, Dowling- |
| | | | | | | | | | | | | | Meara/Kobner/Weber- |
| | | | | | | | | | | | | | Cockayne types) |
| 235751_s_at | −1.76 | 0.0148 | 2.60 | 9.61 | 5.46 | 0.04 | 0.03 | −0.25 | 0.19 | 0.1813 | AA977975 | VMO1 | Vitelline membrane outer |
| | | | | | | | | | | | | | layer 1 homolog (chicken) |
| 206259_at | −1.76 | 0.0122 | 2.68 | 9.61 | 5.46 | 0.04 | 0.03 | −0.25 | 0.17 | 0.1374 | NM_000312 | PROC | Protein C (inactivator of |
| | | | | | | | | | | | | | coagulation factors Va and |
| | | | | | | | | | | | | | VIIIa) |
| 1557611_at | −1.76 | 0.0313 | 2.27 | 15.22 | 8.63 | 0.07 | 0.04 | −0.25 | 0.10 | 0.0085 | AW779022 | TTLL4 | Tubulin tyrosine ligase- |
| | | | | | | | | | | | | | like family, member 4 |
| 203440_at | −1.77 | 0.0836 | 1.80 | 73.41 | 41.58 | 0.34 | 0.19 | −0.25 | 0.17 | 0.1380 | M34064 | CDH2 | Cadherin 2, type 1, N- |
| | | | | | | | | | | | | | cadherin (neuronal) |
| 234034_at | −1.77 | 0.0045 | 3.10 | 8.15 | 4.61 | 0.04 | 0.02 | −0.25 | 0.13 | 0.0416 | AL137510 | KCNMB4 | Potassium large |
| | | | | | | | | | | | | | conductance calcium- |
| | | | | | | | | | | | | | activated channel, |
| | | | | | | | | | | | | | subfamily M, beta member 4 |
| 227529_s_at | −1.77 | 0.0178 | 2.54 | 42.15 | 23.81 | 0.19 | 0.11 | −0.25 | 0.10 | 0.0121 | BF511276 | AKAP12 | A kinase (PRKA) anchor |
| | | | | | | | | | | | | | protein (gravin) 12 |
| 238638_at | −1.78 | 0.0116 | 2.72 | 39.12 | 22.02 | 0.18 | 0.10 | −0.25 | 0.11 | 0.0195 | AI935644 | SLC37A2 | Solute carrier family 37 |
| | | | | | | | | | | | | | (glycerol-3-phosphate |
| | | | | | | | | | | | | | transporter), member 2 |
| 234914_at | −1.78 | 0.0389 | 2.17 | 7.03 | 3.96 | 0.03 | 0.02 | −0.25 | 0.13 | 0.0507 | AK022466 | ZNF7 | Zinc finger protein 7 |
| 243986_at | −1.78 | 0.1242 | 1.62 | 7.88 | 4.43 | 0.04 | 0.02 | −0.25 | 0.27 | 0.3568 | BE409452 | LOC144766 | hypothetical protein |
| | | | | | | | | | | | | | LOC144766 |
| 210037_s_at | −1.78 | 0.0783 | 1.84 | 10.32 | 5.80 | 0.05 | 0.03 | −0.25 | 0.20 | 0.2008 | L24553 | NOS2A | Nitric oxide synthase 2A |
| | | | | | | | | | | | | | (inducible, hepatocytes) |
| 203815_at | −1.78 | 0.1376 | 1.53 | 24.53 | 13.78 | 0.11 | 0.06 | −0.25 | 0.23 | 0.2606 | NM_000853 | GSTT1 | Glutathione S-transferase |
| | | | | | | | | | | | | | theta 1 |
| 219779_at | −1.78 | 0.2553 | 1.17 | 7.13 | 4.00 | 0.03 | 0.02 | −0.25 | 0.22 | 0.2465 | NM_024721 | ZFHX4 | Zinc finger homeodomain 4 |
| 241783_at | −1.78 | 0.0236 | 2.41 | 18.26 | 10.23 | 0.08 | 0.05 | −0.25 | 0.14 | 0.0672 | AI826833 | 241783_at | Transcribed locus |
| 236576_at | −1.79 | 0.2521 | 1.18 | 7.38 | 4.13 | 0.03 | 0.02 | −0.25 | 0.20 | 0.1969 | N63005 | 236576_at | Transcribed locus |
| 232423_at | −1.79 | 0.3201 | 1.01 | 18.75 | 10.50 | 0.09 | 0.05 | −0.25 | 0.17 | 0.1236 | AU144083 | ARSD | Arylsulfatase D |
| 229719_s_at | −1.79 | 0.0277 | 2.34 | 17.55 | 9.80 | 0.08 | 0.05 | −0.25 | 0.14 | 0.0773 | BF433930 | DERL3 | Der1-like domain family, |
| | | | | | | | | | | | | | member 3 |
| 211188_at | −1.80 | 0.6285 | 0.49 | 9.66 | 5.38 | 0.04 | 0.02 | −0.25 | 0.28 | 0.3766 | U96627 | CD84 | CD84 molecule |
| 228525_at | −1.80 | 0.0098 | 2.79 | 8.66 | 4.82 | 0.04 | 0.02 | −0.25 | 0.12 | 0.0334 | AL583533 | SLC7A10 | Transcribed locus |
| 220532_s_at | −1.80 | 0.0620 | 1.95 | 576.06 | 319.62 | 2.65 | 1.48 | −0.26 | 0.12 | 0.0318 | NM_014020 | LR8 | LR8 protein |
| 229148_at | −1.80 | 0.0622 | 1.97 | 8.01 | 4.44 | 0.04 | 0.02 | −0.26 | 0.18 | 0.1661 | AK022839 | HOXA2 | Homo sapiens, clone |
| | | | | | | | | | | | | | IMAGE: 5019307, mRNA |
| 209550_at | −1.81 | 0.1241 | 1.59 | 37.47 | 20.75 | 0.17 | 0.10 | −0.26 | 0.16 | 0.0965 | U35139 | NDN | Necdin homolog (mouse) |
| 1570536_at | −1.81 | 0.1522 | 1.49 | 7.24 | 4.01 | 0.03 | 0.02 | −0.26 | 0.18 | 0.1492 | AY037163 | ribosomal | Homo sapiens ribosomal |
| | | | | | | | | | | | | protein L15 | protein L15 mRNA, |
| | | | | | | | | | | | | | complete cds. |
| 206187_at | −1.82 | 0.1368 | 1.53 | 14.23 | 7.83 | 0.07 | 0.04 | −0.26 | 0.23 | 0.2373 | NM_000960 | PTGIR | Prostaglandin I2 |
| | | | | | | | | | | | | | (prostacyclin) receptor (IP) |
| 209706_at | −1.82 | 0.2972 | 1.06 | 76.52 | 42.08 | 0.35 | 0.20 | −0.26 | 0.24 | 0.2732 | AF247704 | NKX3-1 | NK3 transcription factor |
| | | | | | | | | | | | | | related, locus 1 |
| | | | | | | | | | | | | | (Drosophila) |
| 224348_s_at | −1.82 | 0.0697 | 1.89 | 18.59 | 10.21 | 0.09 | 0.05 | −0.26 | 0.17 | 0.1151 | AF116709 | PRO2605 | Homo sapiens PRO2605 |
| | | | | | | | | | | | | | mRNA, complete cds. |
| 1564052_at | −1.82 | 0.1115 | 1.65 | 524.94 | 287.92 | 2.42 | 1.34 | −0.26 | 0.17 | 0.1157 | AK090633 | TREML4 | Triggering receptor |
| | | | | | | | | | | | | | expressed on myeloid |
| | | | | | | | | | | | | | cells-like 4 |
| 241117_at | −1.83 | 0.0209 | 2.45 | 7.62 | 4.17 | 0.04 | 0.02 | −0.26 | 0.18 | 0.1547 | AW196588 | LOXHD1 | Lipoxygenase homology |
| | | | | | | | | | | | | | domains 1 |
| 1553952_at | −1.83 | 0.1311 | 1.56 | 24.65 | 13.47 | 0.11 | 0.06 | −0.26 | 0.21 | 0.2068 | NM_001039617 | ZDHHC19 | Zinc finger, DHHC-type |
| | | | | | | | | | | | | | containing 19 |
| 218965_s_at | −1.84 | 0.0179 | 2.52 | 19.68 | 10.71 | 0.09 | 0.05 | −0.26 | 0.13 | 0.0299 | NM_022830 | RBM21 | RNA binding motif protein |
| | | | | | | | | | | | | | 21 |
| 223565_at | −1.84 | 0.2732 | 1.12 | 16.53 | 8.99 | 0.08 | 0.04 | −0.26 | 0.30 | 0.3856 | AF151024 | PACAP | proapoptotic caspase |
| | | | | | | | | | | | | | adaptor protein |
| 204695_at | −1.84 | 0.0170 | 2.54 | 16.21 | 8.81 | 0.07 | 0.04 | −0.27 | 0.14 | 0.0584 | AI343459 | CDC25A | Cell division cycle 25A |
| 1560784_x_at | −1.84 | 0.1282 | 1.59 | 16.96 | 9.21 | 0.08 | 0.04 | −0.27 | 0.20 | 0.1939 | BC035326 | 1560784_x_at | CDNA clone |
| | | | | | | | | | | | | | IMAGE: 5186324 |
| 230301_at | −1.85 | 0.0178 | 2.53 | 10.16 | 5.48 | 0.05 | 0.03 | −0.27 | 0.19 | 0.1264 | BF055370 | MGC72075 | hypothetical protein |
| | | | | | | | | | | | | | MGC72075 |
| 215035_at | −1.86 | 0.1590 | 1.45 | 11.31 | 6.08 | 0.05 | 0.03 | −0.27 | 0.21 | 0.1944 | AI952772 | IGLC1 | Immunoglobulin anti- |
| | | | | | | | | | | | | | HBsAg lambda light chain |
| | | | | | | | | | | | | | (LM25) |
| 1562051_at | −1.86 | 0.0179 | 2.53 | 112.92 | 60.59 | 0.52 | 0.28 | −0.27 | 0.11 | 0.0125 | AK092805 | LOC284757 | hypothetical protein |
| | | | | | | | | | | | | | LOC284757 |
| 203706_s_at | −1.87 | 0.0982 | 1.71 | 8.52 | 4.56 | 0.04 | 0.02 | −0.27 | 0.24 | 0.2455 | NM_003507 | FZD7 | Frizzled homolog 7 |
| | | | | | | | | | | | | | (Drosophila) |
| 207517_at | −1.87 | 0.5498 | 0.61 | 7.78 | 4.17 | 0.04 | 0.02 | −0.27 | 0.29 | 0.3577 | NM_018891 | LAMC2 | Laminin, gamma 2 |
| 206771_at | −1.87 | 0.0070 | 2.92 | 25.31 | 13.55 | 0.12 | 0.06 | −0.27 | 0.11 | 0.0069 | NM_006953 | UPK3A | Uroplakin 3A |
| 224397_s_at | −1.88 | 0.2391 | 1.21 | 51.36 | 27.25 | 0.24 | 0.13 | −0.28 | 0.19 | 0.1391 | AF319520 | TMTC1 | Transmembrane and |
| | | | | | | | | | | | | | tetratricopeptide repeat |
| | | | | | | | | | | | | | containing 1 |
| 224049_at | −1.88 | 0.0516 | 2.04 | 12.89 | 6.84 | 0.06 | 0.03 | −0.28 | 0.13 | 0.0253 | AF339912 | KCNK17 | Potassium channel, |
| | | | | | | | | | | | | | subfamily K, member 17 |
| 203324_s_at | −1.89 | 0.1210 | 1.60 | 15.11 | 7.99 | 0.07 | 0.04 | −0.28 | 0.19 | 0.1472 | NM_001233 | CAV2 | Caveolin 2 |
| 209094_at | −1.89 | 0.0203 | 2.46 | 7.12 | 3.76 | 0.03 | 0.02 | −0.28 | 0.15 | 0.0600 | AL078459 | DDAH1 | Human DNA sequence |
| | | | | | | | | | | | | | from clone RP4-621F18 on |
| | | | | | | | | | | | | | chromosome 1p11.4-21.3 |
| | | | | | | | | | | | | | Contains part of a novel |
| | | | | | | | | | | | | | gene, and the 3′ end of the |
| | | | | | | | | | | | | | DDAH1 gene for |
| | | | | | | | | | | | | | dimethylarginine |
| | | | | | | | | | | | | | dimethylaminohydrolase 1, |
| | | | | | | | | | | | | | complete sequence. |
| 218006_s_at | −1.90 | 0.6744 | 0.43 | 23.63 | 12.43 | 0.11 | 0.06 | −0.28 | 0.36 | 0.4404 | NM_006963 | ZNF22 | Zinc finger protein 22 |
| | | | | | | | | | | | | | (KOX 15) |
| 235965_at | −1.91 | 0.0580 | 2.00 | 19.63 | 10.29 | 0.09 | 0.05 | −0.28 | 0.15 | 0.0673 | BF513674 | BHLHB8 | Basic helix-loop-helix |
| | | | | | | | | | | | | | domain containing, class |
| | | | | | | | | | | | | | B, 8 |
| 1555826_at | −1.91 | 0.0514 | 2.07 | 68.14 | 35.67 | 0.31 | 0.17 | −0.28 | 0.19 | 0.1318 | BQ021146 | BIRC5 | CDNA clone |
| | | | | | | | | | | | | | IMAGE: 3354269, |
| | | | | | | | | | | | | | containing frame-shift |
| | | | | | | | | | | | | | errors |
| 235077_at | −1.91 | 0.0202 | 2.47 | 8.02 | 4.20 | 0.04 | 0.02 | −0.28 | 0.13 | 0.0242 | BF956762 | MEG3 | Maternally expressed 3 |
| 221572_s_at | −1.92 | 0.0252 | 2.37 | 30.23 | 15.78 | 0.14 | 0.07 | −0.28 | 0.13 | 0.0213 | AF288410 | SLC26A6 | Solute carrier family 26, |
| | | | | | | | | | | | | | member 6 |
| 243087_at | −1.92 | 0.2018 | 1.31 | 8.83 | 4.60 | 0.04 | 0.02 | −0.28 | 0.24 | 0.2408 | AI860874 | WDR63 | WD repeat domain 63 |
| 1562005_at | −1.92 | 0.3358 | 0.98 | 8.55 | 4.44 | 0.04 | 0.02 | −0.28 | 0.16 | 0.0627 | BC034153 | RIN3 | Homo sapiens Ras and |
| | | | | | | | | | | | | | Rab interactor 3, mRNA |
| | | | | | | | | | | | | | (cDNA clone |
| | | | | | | | | | | | | | IMAGE: 4704191), with |
| | | | | | | | | | | | | | apparent retained intron. |
| 236988_x_at | −1.93 | 0.6123 | 0.51 | 278.94 | 144.62 | 1.29 | 0.67 | −0.29 | 0.23 | 0.2183 | W68403 | ITGB2 | Integrin, beta 2 |
| | | | | | | | | | | | | | (complement component 3 |
| | | | | | | | | | | | | | receptor 3 and 4 subunit) |
| 1562587_at | −1.93 | 0.1477 | 1.51 | 10.33 | 5.35 | 0.05 | 0.02 | −0.29 | 0.27 | 0.2913 | AK093001 | MIST | mast cell immunoreceptor |
| | | | | | | | | | | | | | signal transducer |
| 213265_at | −1.93 | 0.3651 | 0.92 | 8.89 | 4.60 | 0.04 | 0.02 | −0.29 | 0.31 | 0.3321 | AI570199 | PGA5 | Pepsinogen 5, group I |
| | | | | | | | | | | | | | (pepsinogen A) |
| 1560978_at | −1.94 | 0.0261 | 2.37 | 41.17 | 21.26 | 0.19 | 0.10 | −0.29 | 0.15 | 0.0608 | AF088044 | 1560978_at | Full length insert cDNA |
| | | | | | | | | | | | | | clone ZD58F01 |
| 238247_at | −1.94 | 0.1214 | 1.61 | 7.13 | 3.68 | 0.03 | 0.02 | −0.29 | 0.21 | 0.1738 | N32157 | TMC3 | Transmembrane channel- |
| | | | | | | | | | | | | | like 3 |
| 240323_at | −1.94 | 0.3051 | 1.05 | 14.22 | 7.34 | 0.07 | 0.03 | −0.29 | 0.22 | 0.1827 | AW204607 | FXYD6 | FXYD domain containing |
| | | | | | | | | | | | | | ion transport regulator 6 |
| 202094_at | −1.94 | 0.0450 | 2.12 | 16.42 | 8.47 | 0.08 | 0.04 | −0.29 | 0.19 | 0.1280 | AA648913 | BIRC5 | Baculoviral IAP repeat- |
| | | | | | | | | | | | | | containing 5 (survivin) |
| 221730_at | −1.94 | 0.1925 | 1.35 | 9.00 | 4.63 | 0.04 | 0.02 | −0.29 | 0.24 | 0.2356 | NM_000393 | COL5A2 | Collagen, type V, alpha 2 |
| 1563453_at | −1.94 | 0.0742 | 1.86 | 7.90 | 4.06 | 0.04 | 0.02 | −0.29 | 0.18 | 0.1064 | AL833544 | SLC24A3 | Solute carrier family 24 |
| | | | | | | | | | | | | | (sodium/potassium/calcium |
| | | | | | | | | | | | | | exchanger), member 3 |
| 1560316_s_at | −1.98 | 0.0691 | 1.89 | 9.97 | 5.03 | 0.05 | 0.02 | −0.30 | 0.23 | 0.1851 | N32168 | GLCCI1 | Glucocorticoid induced |
| | | | | | | | | | | | | | transcript 1 |
| 226322_at | −1.99 | 0.0872 | 1.77 | 248.69 | 124.94 | 1.15 | 0.58 | −0.30 | 0.17 | 0.0728 | BF109231 | TMTC1 | Transmembrane and |
| | | | | | | | | | | | | | tetratricopeptide repeat |
| | | | | | | | | | | | | | containing 1 |
| 218345_at | −2.01 | 0.0269 | 2.34 | 572.00 | 284.96 | 2.64 | 1.32 | −0.30 | 0.14 | 0.0230 | NM_018487 | HCA112 | hepatocellular carcinoma- |
| | | | | | | | | | | | | | associated antigen 112 |
| 217207_s_at | −2.01 | 0.5897 | 0.55 | 259.73 | 129.16 | 1.20 | 0.60 | −0.30 | 0.30 | 0.3015 | AK025267 | BTNL3 | Butyrophilin-like 3 |
| 217688_at | −2.02 | 0.1523 | 1.50 | 12.78 | 6.32 | 0.06 | 0.03 | −0.31 | 0.25 | 0.2330 | BE677757 | ADCY2 | Adenylate cyclase 2 |
| | | | | | | | | | | | | | (brain) |
| 207802_at | −2.04 | 0.1813 | 1.37 | 208.81 | 102.41 | 0.96 | 0.48 | −0.31 | 0.24 | 0.1880 | NM_006061 | CRISP3 | Cysteine-rich secretory |
| | | | | | | | | | | | | | protein 3 |
| 239970_at | −2.04 | 0.0048 | 3.08 | 7.37 | 3.61 | 0.03 | 0.02 | −0.31 | 0.16 | 0.0386 | AI088361 | 239970_at | Transcribed locus |
| 239048_at | −2.05 | 0.0005 | 3.96 | 7.59 | 3.71 | 0.03 | 0.02 | −0.31 | 0.11 | 0.0041 | BG427399 | KIF1B | Kinesin family member 1B |
| 208335_s_at | −2.05 | 0.0023 | 3.41 | 35.41 | 17.31 | 0.16 | 0.08 | −0.31 | 0.15 | 0.0253 | NM_002036 | DARC | Duffy blood group, |
| | | | | | | | | | | | | | chemokine receptor |
| 214209_s_at | −2.05 | 0.0069 | 2.97 | 12.69 | 6.18 | 0.06 | 0.03 | −0.31 | 0.16 | 0.0551 | BE504895 | ABCB9 | ATP-binding cassette, sub- |
| | | | | | | | | | | | | | family B (MDR/TAP), |
| | | | | | | | | | | | | | member 9 |
| 239591_at | −2.05 | 0.1228 | 1.59 | 179.47 | 87.39 | 0.83 | 0.41 | −0.31 | 0.27 | 0.2254 | BF433269 | 239591_at | Transcribed locus, |
| | | | | | | | | | | | | | moderately similar to |
| | | | | | | | | | | | | | NP_858931.1 NFS1 |
| | | | | | | | | | | | | | nitrogen fixation 1 isoform |
| | | | | | | | | | | | | | b precursor; cysteine |
| | | | | | | | | | | | | | desulfurase; nitrogen- |
| | | | | | | | | | | | | | fixing bacteria S-like |
| | | | | | | | | | | | | | protein; nitrogen fixation 1 |
| | | | | | | | | | | | | | (S. cerevisiae, homolog) |
| | | | | | | | | | | | | | [Homo sapiens] |
| 207641_at | −2.06 | 0.0024 | 3.34 | 16.17 | 7.87 | 0.07 | 0.04 | −0.31 | 0.12 | 0.0054 | NM_012452 | TNFRSF13B | Tumor necrosis factor |
| | | | | | | | | | | | | | receptor superfamily, |
| | | | | | | | | | | | | | member 13B |
| 224753_at | −2.06 | 0.0377 | 2.24 | 19.63 | 9.51 | 0.09 | 0.04 | −0.31 | 0.17 | 0.0666 | BE614410 | CDCA5 | Cell division cycle |
| | | | | | | | | | | | | | associated 5 |
| 238900_at | −2.09 | 0.1417 | 1.51 | 63.17 | 30.22 | 0.29 | 0.14 | −0.32 | 0.29 | 0.2435 | BE669692 | HLA-DQB1 | Transcribed locus |
| 231223_at | −2.11 | 0.4490 | 0.77 | 14.24 | 6.76 | 0.07 | 0.03 | −0.32 | 0.32 | 0.3064 | R41565 | CSMD1 | CUB and Sushi multiple |
| | | | | | | | | | | | | | domains 1 |
| 202065_s_at | −2.11 | 0.0655 | 1.94 | 9.58 | 4.55 | 0.04 | 0.02 | −0.32 | 0.20 | 0.0986 | BG033593 | PPFIA1 | 602301717F1 |
| | | | | | | | | | | | | | NIH_MGC_87 Homo |
| | | | | | | | | | | | | | sapiens cDNA clone |
| | | | | | | | | | | | | | IMAGE: 4403212 5′, |
| | | | | | | | | | | | | | mRNA sequence. |
| 240573_at | −2.11 | 0.2803 | 1.10 | 72.22 | 34.17 | 0.33 | 0.16 | −0.33 | 0.28 | 0.2313 | BF436632 | LOC374443 | CLR pseudogene |
| 231416_at | −2.12 | 0.1139 | 1.63 | 7.77 | 3.67 | 0.04 | 0.02 | −0.33 | 0.21 | 0.1087 | NM_014475 | DHDH | Dihydrodiol |
| | | | | | | | | | | | | | dehydrogenase (dimeric) |
| 203149_at | −2.12 | 0.4227 | 0.82 | 29.84 | 14.05 | 0.14 | 0.07 | −0.33 | 0.32 | 0.3048 | NM_002856 | PVRL2 | Translocase of outer |
| | | | | | | | | | | | | | mitochondrial membrane |
| | | | | | | | | | | | | | 40 homolog (yeast) |
| 215036_at | −2.13 | 0.2353 | 1.22 | 26.41 | 12.42 | 0.12 | 0.06 | −0.33 | 0.26 | 0.2125 | AI952772 | IGLC1 | Immunoglobulin anti- |
| | | | | | | | | | | | | | HBsAg lambda light chain |
| | | | | | | | | | | | | | (LM25) |
| 219050_s_at | −2.13 | 0.0100 | 2.77 | 8.97 | 4.21 | 0.04 | 0.02 | −0.33 | 0.15 | 0.0193 | NM_014205 | ZNHIT2 | Zinc finger, HIT type 2 |
| 1557165_s_at | −2.14 | 0.0065 | 2.94 | 15.01 | 7.01 | 0.07 | 0.03 | −0.33 | 0.17 | 0.0346 | BM141828 | KLHL18 | Kelch-like 18 (Drosophila) |
| 232079_s_at | −2.17 | 0.1833 | 1.38 | 19.27 | 8.89 | 0.09 | 0.04 | −0.34 | 0.33 | 0.3006 | BE867789 | TOMM40 | Translocase of outer |
| | | | | | | | | | | | | | mitochondrial membrane |
| | | | | | | | | | | | | | 40 homolog (yeast) |
| 207106_s_at | −2.17 | 0.0528 | 2.02 | 25.92 | 11.95 | 0.12 | 0.06 | −0.34 | 0.17 | 0.0462 | NM_002344 | LTK | Leukocyte tyrosine kinase |
| 206588_at | −2.19 | 0.3311 | 0.99 | 8.57 | 3.91 | 0.04 | 0.02 | −0.34 | 0.39 | 0.3799 | NM_001351 | DAZL | Deleted in azoospermia- |
| | | | | | | | | | | | | | like |
| 226931_at | −2.20 | 0.3181 | 1.02 | 31.54 | 14.37 | 0.15 | 0.07 | −0.34 | 0.24 | 0.1462 | AU151239 | TMTC1 | Transmembrane and |
| | | | | | | | | | | | | | tetratricopeptide repeat |
| | | | | | | | | | | | | | containing 1 |
| 237120_at | −2.20 | 0.8831 | −0.15 | 21.18 | 9.62 | 0.10 | 0.04 | −0.34 | 0.66 | 0.5982 | AI186548 | KRT77 | Keratin 1B |
| 231381_at | −2.20 | 0.4137 | 0.83 | 39.94 | 18.14 | 0.18 | 0.08 | −0.34 | 0.31 | 0.2581 | BF223023 | CACNA2D3 | Calcium channel, voltage- |
| | | | | | | | | | | | | | dependent, alpha 2/delta 3 |
| | | | | | | | | | | | | | subunit |
| 232598_at | −2.21 | 0.3758 | 0.91 | 8.35 | 3.77 | 0.04 | 0.02 | −0.35 | 0.33 | 0.2881 | AL133633 | NUP210L | Nucleoporin 210 kDa-like |
| 210517_s_at | −2.22 | 0.0015 | 3.52 | 44.26 | 19.94 | 0.20 | 0.09 | −0.35 | 0.09 | 0.0001 | AB003476 | AKAP12 | A kinase (PRKA) anchor |
| | | | | | | | | | | | | | protein (gravin) 12 |
| 219825_at | −2.39 | 0.2662 | 1.13 | 18.68 | 7.83 | 0.09 | 0.04 | −0.38 | 0.44 | 0.3648 | NM_019885 | CYP26B1 | Cytochrome P450, family |
| | | | | | | | | | | | | | 26, subfamily B, |
| | | | | | | | | | | | | | polypeptide 1 |
| 201785_at | −2.41 | 0.8959 | 0.13 | 20.70 | 8.60 | 0.10 | 0.04 | −0.38 | 0.45 | 0.4006 | NM_002933 | RNASE1 | Ribonuclease, RNase A |
| | | | | | | | | | | | | | family, 1 (pancreatic) |
| 203559_s_at | −2.50 | 0.0305 | 2.28 | 54.30 | 21.68 | 0.25 | 0.10 | −0.40 | 0.22 | 0.0569 | NM_001091 | ABP1 | Potassium voltage-gated |
| | | | | | | | | | | | | | channel, subfamily H (eag- |
| | | | | | | | | | | | | | related), member 2 |
| 205493_s_at | −2.58 | 0.2539 | 1.17 | 11.14 | 4.32 | 0.05 | 0.02 | −0.41 | 0.28 | 0.1083 | NM_006426 | DPYSL4 | Dihydropyrimidinase-like 4 |
| 219669_at | −2.63 | 0.0025 | 3.33 | 427.21 | 162.51 | 1.97 | 0.75 | −0.42 | 0.15 | 0.0034 | NM_020406 | CD177 | gb: NM_020406.1 |
| | | | | | | | | | | | | | /DEF = Homo sapiens |
| | | | | | | | | | | | | | polycythemia rubra vera 1; |
| | | | | | | | | | | | | | cell surface receptor |
| | | | | | | | | | | | | | (PRV1), mRNA. |
| | | | | | | | | | | | | | /FEA = mRNA |
| | | | | | | | | | | | | | /GEN = PRV1 |
| | | | | | | | | | | | | | /PROD = polycythemia |
| | | | | | | | | | | | | | rubra vera 1; cell |
| | | | | | | | | | | | | | surfacereceptor |
| | | | | | | | | | | | | | /DB_XREF = gi: 9966888 |
| | | | | | | | | | | | | | /UG = Hs.232165 |
| | | | | | | | | | | | | | polycythemia rubra vera 1; |
| | | | | | | | | | | | | | cell surface receptor |
| | | | | | | | | | | | | | /FL = gb: AF146747.1 |
| | | | | | | | | | | | | | gb: NM_020406.1 |
| 227019_at | −2.82 | 0.4594 | 0.76 | 11.64 | 4.12 | 0.05 | 0.02 | −0.45 | 0.51 | 0.3691 | AA129774 | FLJ13137 | hypothetical gene |
| | | | | | | | | | | | | | supported by AK125122 |
| 223906_s_at | −2.98 | 0.0050 | 3.06 | 9.99 | 3.35 | 0.05 | 0.02 | −0.47 | 0.24 | 0.0418 | AY014285 | TEX101 | Testis expressed sequence |
| | | | | | | | | | | | | | 101 |
| 205514_at | −3.11 | 0.3847 | 0.89 | 21.80 | 7.00 | 0.10 | 0.03 | −0.49 | 0.56 | 0.3655 | NM_018355 | ZNF415 | Zinc finger protein 415 |
| 205843_x_at | −3.22 | 0.0545 | 2.01 | 8.92 | 2.77 | 0.04 | 0.01 | −0.51 | 0.32 | 0.0604 | NM_000755 | CRAT | Carnitine acetyltransferase |
| 225418_at | −3.86 | 0.1938 | 1.35 | 62.37 | 16.17 | 0.29 | 0.08 | −0.59 | 0.41 | 0.1387 | AI520949 | TOMM40 | Translocase of outer |
| | | | | | | | | | | | | | mitochondrial membrane |
| | | | | | | | | | | | | | 40 homolog (yeast) |
| 219082_at | −4.01 | 0.0183 | 2.51 | 8.89 | 2.22 | 0.04 | 0.01 | −0.60 | 0.30 | 0.0104 | NM_015944 | AMDHD2 | Amidohydrolase domain |
| | | | | | | | | | | | | | containing 2 |
| 232731_x_at | −4.15 | 0.6594 | 0.45 | 70.88 | 17.09 | 0.33 | 0.08 | −0.62 | 0.08 | 0.0000 | BC001607 | RAMP2 | Receptor (calcitonin) |
| | | | | | | | | | | | | | activity modifying protein 2 |
| 241234_at | −4.45 | 0.1538 | 1.49 | 18.85 | 4.24 | 0.09 | 0.02 | −0.65 | 0.44 | 0.1066 | AA993387 | LOC645733 | Transcribed locus, strongly |
| | | | | | | | | | | | | | similar to XP_374004.1 |
| | | | | | | | | | | | | | PREDICTED: hypothetical |
| | | | | | | | | | | | | | protein XP_374004 [Homo |
| | | | | | | | | | | | | | sapiens] |
| 207748_at | −10.27 | 0.6437 | 0.47 | 30.50 | 2.97 | 0.14 | 0.01 | −1.01 | 0.17 | 0.0000 | NM_001349 | DARS | Aspartyl-tRNA synthetase |

TABLE 2
253 Genes for Preterm Delivery

| | | | | | Control | Preterm | | | | | | |
|---|---|---|---|---|---|---|---|---|---|---|---|---|
| | | t-test | Control | Preterm | (norm) | (norm) | Log | Log | P-value | | Sequence | |
| Affy Probe IDs | Fold Change | P-value | Intensity | Intensity | Intensity | Intensity | (Ratio) | (Error) | Resolver | Accession # | Name | Sequence Description |
| 206778_at | 2.97 | 0.0013 | 5.84 | 17.36 | 0.03 | 0.08 | 0.47 | 0.15 | 0.0016 | NM_000496 | CRYBB2 | Crystallin, beta B2 |
| 211613_s_at | 2.23 | 0.0051 | 5.46 | 12.18 | 0.03 | 0.06 | 0.35 | 0.14 | 0.0111 | U79250 | GPD2 | Glycerol-3-phosphate |
| | | | | | | | | | | | | dehydrogenase 2 |
| | | | | | | | | | | | | (mitochondrial) |
| 207891_s_at | 2.10 | 0.0018 | 3.78 | 7.96 | 0.02 | 0.04 | 0.32 | 0.11 | 0.0035 | NM_017518 | UIP1 | 26S proteasome-associated |
| | | | | | | | | | | | | UCH interacting protein 1 |
| 237009_at | 2.10 | 0.0088 | 8.48 | 17.82 | 0.04 | 0.08 | 0.32 | 0.15 | 0.0311 | BF439675 | CD69 | CD69 molecule |
| 205138_s_at | 2.05 | 0.0045 | 4.21 | 8.62 | 0.02 | 0.04 | 0.31 | 0.14 | 0.0187 | AW418882 | UST | Uronyl-2-sulfotransferase |
| 231313_at | 2.04 | 0.0038 | 3.75 | 7.65 | 0.02 | 0.04 | 0.31 | 0.13 | 0.0160 | AW134984 | LRRC8B | Leucine rich repeat |
| | | | | | | | | | | | | containing 8 family, |
| | | | | | | | | | | | | member B |
| 1557610_at | 2.01 | 0.0048 | 5.60 | 11.23 | 0.03 | 0.05 | 0.30 | 0.11 | 0.0051 | AI003930 | PITRM1 | Pitrilysin metallopeptidase 1 |
| 237606_at | 1.82 | 0.0078 | 4.98 | 9.04 | 0.02 | 0.04 | 0.26 | 0.09 | 0.0056 | AI022073 | CD53 | CD53 molecule |
| 240803_at | 1.79 | 0.0069 | 5.00 | 8.95 | 0.02 | 0.04 | 0.25 | 0.12 | 0.0400 | AW450626 | C1orf131 | Chromosome 1 open |
| | | | | | | | | | | | | reading frame 131 |
| 205717_x_at | 1.77 | 0.0019 | 4.85 | 8.61 | 0.02 | 0.04 | 0.25 | 0.10 | 0.0138 | NM_002588 | PCDHGC3 | Protocadherin gamma |
| | | | | | | | | | | | | subfamily C, 3 |
| 238635_at | 1.76 | 0.0055 | 6.69 | 11.77 | 0.03 | 0.05 | 0.25 | 0.09 | 0.0099 | W72333 | FLJ21657 | hypothetical protein |
| | | | | | | | | | | | | FLJ21657 |
| 1566645_at | 1.76 | 0.0058 | 7.79 | 13.68 | 0.04 | 0.06 | 0.24 | 0.11 | 0.0256 | AL050106 | NHEJ1 | Nonhomologous end- |
| | | | | | | | | | | | | joining factor 1 |
| 216965_x_at | 1.74 | 0.0065 | 5.71 | 9.96 | 0.03 | 0.05 | 0.24 | 0.12 | 0.0445 | AL139377 | SPG20 | Human DNA sequence |
| | | | | | | | | | | | | from clone RP11-251J8 on |
| | | | | | | | | | | | | chromosome 13 Contains 2 |
| | | | | | | | | | | | | novel genes, the KIAA0610 |
| | | | | | | | | | | | | gene and a CpG island, |
| | | | | | | | | | | | | complete sequence. |
| 217374_x_at | 1.74 | 0.0010 | 14.52 | 25.30 | 0.07 | 0.12 | 0.24 | 0.12 | 0.0374 | AC006033 | STARD3NL | Homo sapiens BAC clone |
| | | | | | | | | | | | | RP11-121A8 from 7, |
| | | | | | | | | | | | | complete sequence. |
| 230996_at | 1.72 | 0.0059 | 6.46 | 11.09 | 0.03 | 0.05 | 0.23 | 0.12 | 0.0428 | AW024499 | LOC339929 | hypothetical protein |
| | | | | | | | | | | | | LOC339929 |
| 238621_at | 1.67 | 0.0044 | 10.97 | 18.28 | 0.05 | 0.08 | 0.22 | 0.10 | 0.0205 | R67695 | FMN1 | Formin 1 |
| 218948_at | 1.66 | 0.0024 | 20.65 | 34.26 | 0.10 | 0.16 | 0.22 | 0.12 | 0.0491 | AL136679 | QRSL1 | Glutaminyl-tRNA synthase |
| | | | | | | | | | | | | (glutamine-hydrolyzing)- |
| | | | | | | | | | | | | like 1 |
| 1567035_at | 1.65 | 0.0037 | 6.04 | 9.94 | 0.03 | 0.05 | 0.22 | 0.11 | 0.0484 | U63828 | C20orf181 | Chromosome 20 open |
| | | | | | | | | | | | | reading frame 181 |
| 233015_at | 1.63 | 0.0090 | 5.10 | 8.34 | 0.02 | 0.04 | 0.21 | 0.11 | 0.0483 | AA732240 | MBNL1 | Muscleblind-like |
| | | | | | | | | | | | | (Drosophila) |
| 243947_s_at | 1.63 | 0.0100 | 9.02 | 14.74 | 0.04 | 0.07 | 0.21 | 0.11 | 0.0564 | AW300612 | 243947_s_at | Transcribed locus |
| 220703_at | 1.63 | 0.0071 | 19.74 | 32.25 | 0.09 | 0.15 | 0.21 | 0.08 | 0.0068 | NM_018470 | C10orf110 | Chromosome 10 open |
| | | | | | | | | | | | | reading frame 110 |
| 202733_at | 1.62 | 0.0027 | 5.12 | 8.27 | 0.02 | 0.04 | 0.21 | 0.13 | 0.0901 | NM_004199 | P4HA2 | Procollagen-proline, 2- |
| | | | | | | | | | | | | oxoglutarate 4-dioxygenase |
| | | | | | | | | | | | | (proline 4-hydroxylase), |
| | | | | | | | | | | | | alpha polypeptide II |
| 234896_at | 1.61 | 0.0053 | 5.02 | 8.09 | 0.02 | 0.04 | 0.21 | 0.09 | 0.0252 | AJ012680 | C1orf5 | Homo sapiens gene |
| | | | | | | | | | | | | encoding hypothetical |
| | | | | | | | | | | | | protein with HTH motif. |
| 242146_at | 1.60 | 0.0049 | 62.32 | 99.88 | 0.29 | 0.46 | 0.20 | 0.07 | 0.0056 | AA872471 | SNRPA1 | Small nuclear |
| | | | | | | | | | | | | ribonucleoprotein |
| | | | | | | | | | | | | polypeptide A′ |
| 226498_at | 1.59 | 0.0085 | 8.44 | 13.46 | 0.04 | 0.06 | 0.20 | 0.09 | 0.0213 | AA149648 | FLT1 | Fms-related tyrosine kinase |
| | | | | | | | | | | | | 1 (vascular endothelial |
| | | | | | | | | | | | | growth factor/vascular |
| | | | | | | | | | | | | permeability factor |
| | | | | | | | | | | | | receptor) |
| 244638_at | 1.56 | 0.0066 | 9.72 | 15.21 | 0.04 | 0.07 | 0.19 | 0.07 | 0.0040 | AW954477 | SUCLG1 | Succinate-CoA ligase, |
| | | | | | | | | | | | | GDP-forming, alpha |
| | | | | | | | | | | | | subunit |
| 242174_at | 1.56 | 0.0009 | 5.00 | 7.79 | 0.02 | 0.04 | 0.19 | 0.07 | 0.0035 | AI732542 | ZBTB10 | ni36g03.x5 |
| | | | | | | | | | | | | NCI_CGAP_Lu1 Homo |
| | | | | | | | | | | | | sapiens cDNA clone |
| | | | | | | | | | | | | IMAGE: 978964 3′ similar |
| | | | | | | | | | | | | to contains Alu repetitive |
| | | | | | | | | | | | | element; contains element |
| | | | | | | | | | | | | TAR1 TAR1 repetitive |
| | | | | | | | | | | | | element; mRNA sequence. |
| 227701_at | 1.55 | 0.0016 | 167.67 | 260.66 | 0.77 | 1.21 | 0.19 | 0.06 | 0.0020 | AK024739 | C10orf118 | Chromosome 10 open |
| | | | | | | | | | | | | reading frame 118 |
| 1559491_at | 1.54 | 0.0048 | 6.59 | 10.13 | 0.03 | 0.05 | 0.19 | 0.11 | 0.0682 | AL390180 | TNRC17 | MRNA; cDNA |
| | | | | | | | | | | | | DKFZp761L149 (from |
| | | | | | | | | | | | | clone DKFZp761L149) |
| 222727_s_at | 1.54 | 0.0062 | 5.04 | 7.74 | 0.02 | 0.04 | 0.19 | 0.07 | 0.0121 | AI339568 | SLC24A6 | Solute carrier family 24 |
| | | | | | | | | | | | | (sodium/potassium/calcium |
| | | | | | | | | | | | | exchanger), member 6 |
| 240114_s_at | 1.53 | 0.0003 | 5.24 | 7.99 | 0.02 | 0.04 | 0.18 | 0.07 | 0.0065 | AI927971 | MGC13034 | hypothetical protein |
| | | | | | | | | | | | | MGC13034 |
| 240539_at | 1.51 | 0.0068 | 6.04 | 9.14 | 0.03 | 0.04 | 0.18 | 0.09 | 0.0367 | AI684551 | AUTS2 | Autism susceptibility |
| | | | | | | | | | | | | candidate 2 |
| 1556439_at | 1.51 | 0.0060 | 4.09 | 6.17 | 0.02 | 0.03 | 0.18 | 0.08 | 0.0218 | AL832163 | LOC441376 | AARD protein |
| 1570038_at | 1.50 | 0.0043 | 13.56 | 20.30 | 0.06 | 0.09 | 0.18 | 0.09 | 0.0583 | BC009008 | ZNF595 | Zinc finger protein 595 |
| 1569362_at | 1.48 | 0.0051 | 5.17 | 7.64 | 0.02 | 0.04 | 0.17 | 0.08 | 0.0261 | BC041127 | ALCAM | Activated leukocyte cell |
| | | | | | | | | | | | | adhesion molecule |
| 235648_at | 1.48 | 0.0044 | 34.70 | 51.21 | 0.16 | 0.24 | 0.17 | 0.06 | 0.0087 | AA742659 | ZNF567 | Zinc finger protein 567 |
| 1566524_a_at | 1.47 | 0.0009 | 5.75 | 8.48 | 0.03 | 0.04 | 0.17 | 0.07 | 0.0126 | AL832712 | VDAC2 | Voltage-dependent anion |
| | | | | | | | | | | | | channel 2 |
| 240327_at | 1.47 | 0.0073 | 10.21 | 14.99 | 0.05 | 0.07 | 0.17 | 0.08 | 0.0302 | AI968130 | 6-Sep | Septin 6 |
| 1562416_at | 1.46 | 0.0026 | 8.22 | 11.99 | 0.04 | 0.06 | 0.16 | 0.09 | 0.0533 | AI524619 | FLNB | Filamin B, beta (actin |
| | | | | | | | | | | | | binding protein 278) |
| 231080_at | 1.45 | 0.0069 | 6.84 | 9.90 | 0.03 | 0.05 | 0.16 | 0.09 | 0.0626 | AI951606 | CDAN1 | Congenital |
| | | | | | | | | | | | | dyserythropoietic anemia, |
| | | | | | | | | | | | | type I |
| 1555464_at | 1.45 | 0.0070 | 18.57 | 26.84 | 0.09 | 0.12 | 0.16 | 0.06 | 0.0065 | BC046208 | IFIH1 | Interferon induced with |
| | | | | | | | | | | | | helicase C domain 1 |
| 1554882_at | 1.45 | 0.0030 | 6.71 | 9.69 | 0.03 | 0.05 | 0.16 | 0.10 | 0.0974 | BC009793 | ERCC8 | Excision repair cross- |
| | | | | | | | | | | | | complementing rodent |
| | | | | | | | | | | | | repair deficiency, |
| | | | | | | | | | | | | complementation group 8 |
| 234610_at | 1.44 | 0.0011 | 5.22 | 7.54 | 0.02 | 0.04 | 0.16 | 0.07 | 0.0295 | AL109804 | HSPA12B | Human DNA sequence |
| | | | | | | | | | | | | from clone RP5-1009E24 |
| | | | | | | | | | | | | on chromosome 20 |
| | | | | | | | | | | | | Contains the 5′ end of the |
| | | | | | | | | | | | | ADAM33 gene for a |
| | | | | | | | | | | | | disintegrin and |
| | | | | | | | | | | | | metalloproteinase domain |
| | | | | | | | | | | | | 33 protein, the SN gene |
| | | | | | | | | | | | | encoding sialoadhesin, the |
| | | | | | | | | | | | | C20orf60 gene, the |
| | | | | | | | | | | | | C20orf27 gene, the |
| | | | | | | | | | | | | C20orf28 gene, the CENPB |
| | | | | | | | | | | | | gene for centromere protein |
| | | | | | | | | | | | | B, the CDC25B gene for |
| | | | | | | | | | | | | cell division cycle protein |
| | | | | | | | | | | | | 25B, the C20orf29 gene, |
| | | | | | | | | | | | | the 5′ end of the gene for a |
| | | | | | | | | | | | | novel protein (KIAA1271) |
| | | | | | | | | | | | | and nine CpG islands, |
| | | | | | | | | | | | | complete sequence. |
| 240022_at | 1.44 | 0.0078 | 6.63 | 9.56 | 0.03 | 0.04 | 0.16 | 0.08 | 0.0593 | AA770059 | C19orf7 | Chromosome 19 open |
| | | | | | | | | | | | | reading frame 7 |
| 1557262_at | 1.44 | 0.0037 | 8.41 | 12.11 | 0.04 | 0.06 | 0.16 | 0.09 | 0.0684 | AK092855 | AP1G2 | CDNA FLJ35536 fis, clone |
| | | | | | | | | | | | | SPLEN2002451 |
| 1569183_a_at | 1.44 | 0.0037 | 18.46 | 26.58 | 0.09 | 0.12 | 0.16 | 0.06 | 0.0135 | BC032237 | CHM | Choroideremia (Rab escort |
| | | | | | | | | | | | | protein 1) |
| 215345_x_at | 1.44 | 0.0086 | 10.55 | 15.19 | 0.05 | 0.07 | 0.16 | 0.09 | 0.0754 | AA310709 | TARP | TCR gamma alternate |
| | | | | | | | | | | | | reading frame protein |
| 216677_at | 1.44 | 0.0012 | 8.74 | 12.54 | 0.04 | 0.06 | 0.16 | 0.07 | 0.0259 | U20648 | ZNF154 | Zinc finger protein 154 |
| | | | | | | | | | | | | (pHZ-92) |
| 1556361_s_at | 1.43 | 0.0067 | 13.08 | 18.72 | 0.06 | 0.09 | 0.16 | 0.07 | 0.0218 | BC016937 | ANKRD13C | Ankyrin repeat domain 13C |
| 1566041_at | 1.42 | 0.0029 | 4.40 | 6.26 | 0.02 | 0.03 | 0.15 | 0.08 | 0.0535 | D61705 | Ets-like | Ets-like protein (clone 2B) |
| | | | | | | | | | | | protein | |
| 231012_at | 1.42 | 0.0018 | 11.13 | 15.79 | 0.05 | 0.07 | 0.15 | 0.07 | 0.0348 | AI123333 | TMEM20 | Transmembrane protein 20 |
| 237165_at | 1.42 | 0.0055 | 10.11 | 14.35 | 0.05 | 0.07 | 0.15 | 0.08 | 0.0513 | AA760860 | RAB14 | RAB14, member RAS |
| | | | | | | | | | | | | oncogene family |
| 232800_at | 1.41 | 0.0042 | 14.33 | 20.22 | 0.07 | 0.09 | 0.15 | 0.07 | 0.0452 | AW086077 | LOC644450 | hypothetical protein |
| | | | | | | | | | | | | LOC644450 |
| 209437_s_at | 1.41 | 0.0084 | 4.43 | 6.23 | 0.02 | 0.03 | 0.15 | 0.07 | 0.0450 | AB051390 | SPON1 | Spondin 1, extracellular |
| | | | | | | | | | | | | matrix protein |
| 230363_s_at | 1.40 | 0.0062 | 13.38 | 18.76 | 0.06 | 0.09 | 0.15 | 0.07 | 0.0259 | BE858808 | INPP5F | Inositol polyphosphate-5- |
| | | | | | | | | | | | | phosphatase F |
| 229478_x_at | 1.40 | 0.0021 | 17.43 | 24.44 | 0.08 | 0.11 | 0.15 | 0.05 | 0.0062 | AW274311 | BIVM | Basic, immunoglobulin-like |
| | | | | | | | | | | | | variable motif containing |
| 1558101_at | 1.40 | 0.0080 | 6.90 | 9.62 | 0.03 | 0.04 | 0.14 | 0.09 | 0.0901 | BM546261 | 1558101_at | Transcribed locus |
| 1554143_a_at | 1.40 | 0.0021 | 13.58 | 18.94 | 0.06 | 0.09 | 0.14 | 0.05 | 0.0043 | BC020814 | SUGT1L1 | Hypothetical protein |
| | | | | | | | | | | | | LOC283507 |
| 205092_x_at | 1.39 | 0.0093 | 89.31 | 124.47 | 0.41 | 0.58 | 0.14 | 0.06 | 0.0197 | NM_014950 | ZBTB1 | Zinc finger and BTB |
| | | | | | | | | | | | | domain containing 1 |
| 1555034_at | 1.39 | 0.0094 | 4.88 | 6.77 | 0.02 | 0.03 | 0.14 | 0.07 | 0.0516 | AF482697 | USH3A | Usher syndrome 3A |
| 223625_at | 1.38 | 0.0088 | 10.98 | 15.20 | 0.05 | 0.07 | 0.14 | 0.07 | 0.0315 | AB030241 | DRCTNNB1A | down-regulated by Ctnnb1, a |
| 1555024_at | 1.38 | 0.0060 | 8.42 | 11.65 | 0.04 | 0.05 | 0.14 | 0.06 | 0.0137 | BC036029 | ADAM22 | ADAM metallopeptidase |
| | | | | | | | | | | | | domain 22 |
| 1552970_s_at | 1.38 | 0.0022 | 6.24 | 8.64 | 0.03 | 0.04 | 0.14 | 0.08 | 0.0691 | NM_007167 | ZMYM6 | Zinc finger, MYM-type 6 |
| 1564008_at | 1.38 | 0.0040 | 4.85 | 6.69 | 0.02 | 0.03 | 0.14 | 0.08 | 0.0751 | BC006310 | COL27A1 | Collagen, type XXVII, |
| | | | | | | | | | | | | alpha 1 |
| 243969_at | 1.38 | 0.0072 | 393.04 | 541.14 | 1.81 | 2.51 | 0.14 | 0.05 | 0.0065 | W90718 | SLC24A4 | Solute carrier family 24 |
| | | | | | | | | | | | | (sodium/potassium/calcium |
| | | | | | | | | | | | | exchanger), member 4 |
| 201844_s_at | 1.37 | 0.0036 | 463.29 | 635.25 | 2.13 | 2.95 | 0.14 | 0.05 | 0.0025 | W84482 | RYBP | RING1 and YY1 binding |
| | | | | | | | | | | | | protein |
| 219728_at | 1.37 | 0.0093 | 22.65 | 30.96 | 0.10 | 0.14 | 0.14 | 0.07 | 0.0380 | NM_006790 | MYOT | Myotilin |
| 221238_at | 1.36 | 0.0086 | 4.78 | 6.52 | 0.02 | 0.03 | 0.13 | 0.07 | 0.0699 | NM_030763 | NSBP1 | Nucleosomal binding |
| | | | | | | | | | | | | protein 1 |
| 227247_at | 1.36 | 0.0003 | 59.87 | 81.34 | 0.28 | 0.38 | 0.13 | 0.04 | 0.0002 | H98994 | PLEKHA8 | Pleckstrin homology |
| | | | | | | | | | | | | domain containing, family |
| | | | | | | | | | | | | A (phosphoinositide |
| | | | | | | | | | | | | binding specific) member 8 |
| 231254_at | 1.36 | 0.0037 | 19.37 | 26.25 | 0.09 | 0.12 | 0.13 | 0.05 | 0.0091 | BG153387 | ZNF141 | nad34a03.x1 |
| | | | | | | | | | | | | NCI_CGAP_Lu24 Homo |
| | | | | | | | | | | | | sapiens cDNA clone |
| | | | | | | | | | | | | IMAGE: 3367204 3′, |
| | | | | | | | | | | | | mRNA sequence. |
| 238243_at | 1.35 | 0.0073 | 32.77 | 44.36 | 0.15 | 0.21 | 0.13 | 0.07 | 0.0475 | AW085501 | CCNH | Cyclin H |
| 1555180_at | 1.35 | 0.0083 | 6.36 | 8.59 | 0.03 | 0.04 | 0.13 | 0.09 | 0.1377 | BC020894 | C11orf34 | Chromosome 11 open |
| | | | | | | | | | | | | reading frame 34 |
| 242731_x_at | 1.35 | 0.0056 | 56.88 | 76.79 | 0.26 | 0.36 | 0.13 | 0.04 | 0.0039 | AI312371 | 242731_x_at | Transcribed locus |
| 206466_at | 1.35 | 0.0065 | 12.64 | 17.06 | 0.06 | 0.08 | 0.13 | 0.06 | 0.0176 | AB014531 | ACSBG1 | Homo sapiens mRNA for |
| | | | | | | | | | | | | KIAA0631 protein, partial |
| | | | | | | | | | | | | cds. |
| 233585_at | 1.34 | 0.0053 | 13.58 | 18.25 | 0.06 | 0.08 | 0.13 | 0.07 | 0.0532 | AB040947 | SDK2 | Sidekick homolog 2 |
| | | | | | | | | | | | | (chicken) |
| 230047_at | 1.34 | 0.0042 | 7.83 | 10.52 | 0.04 | 0.05 | 0.13 | 0.06 | 0.0293 | BF439533 | FLJ32810 | hypothetical protein |
| | | | | | | | | | | | | FLJ32810 |
| 238501_at | 1.34 | 0.0072 | 26.86 | 36.08 | 0.12 | 0.17 | 0.13 | 0.04 | 0.0036 | AA992936 | 238501_at | Transcribed locus |
| 211148_s_at | 1.34 | 0.0086 | 5.11 | 6.84 | 0.02 | 0.03 | 0.13 | 0.08 | 0.1161 | AF187858 | ANGPT2 | Angiopoietin 2 |
| 217630_at | 1.34 | 0.0015 | 37.63 | 50.37 | 0.17 | 0.23 | 0.13 | 0.06 | 0.0445 | AI188346 | ANGEL2 | Angel homolog 2 |
| | | | | | | | | | | | | (Drosophila) |
| 238555_at | 1.34 | 0.0031 | 10.09 | 13.48 | 0.05 | 0.06 | 0.13 | 0.04 | 0.0033 | AW007410 | MRPS31 | Mitochondrial ribosomal |
| | | | | | | | | | | | | protein S31 |
| 203810_at | 1.33 | 0.0007 | 42.07 | 55.89 | 0.19 | 0.26 | 0.12 | 0.04 | 0.0010 | BG252490 | DNAJB4 | DnaJ (Hsp40) homolog, |
| | | | | | | | | | | | | subfamily B, member 4 |
| 220697_at | 1.32 | 0.0072 | 11.26 | 14.90 | 0.05 | 0.07 | 0.12 | 0.06 | 0.0573 | XM_931774 | LOC643749 | hypothetical protein |
| | | | | | | | | | | | | LOC643749 |
| 1566987_s_at | 1.30 | 0.0080 | 21.67 | 28.27 | 0.10 | 0.13 | 0.12 | 0.04 | 0.0025 | AL137380 | SH3GLP3 | MRNA; cDNA |
| | | | | | | | | | | | | DKFZp434K0626 (from |
| | | | | | | | | | | | | clone DKFZp434K0626) |
| 212079_s_at | 1.30 | 0.0049 | 7.50 | 9.78 | 0.03 | 0.05 | 0.12 | 0.07 | 0.0884 | AA715041 | MLL | nx94c09.s1 |
| | | | | | | | | | | | | NCI_CGAP_GCB1 Homo |
| | | | | | | | | | | | | sapiens cDNA clone |
| | | | | | | | | | | | | IMAGE: 1269904 3′ similar |
| | | | | | | | | | | | | to gb: L04284 ZINC |
| | | | | | | | | | | | | FINGER PROTEIN HRX |
| | | | | | | | | | | | | (HUMAN); mRNA |
| | | | | | | | | | | | | sequence. |
| 227176_at | 1.30 | 0.0021 | 23.28 | 30.34 | 0.11 | 0.14 | 0.12 | 0.04 | 0.0029 | AL565362 | SLC2A13 | Solute carrier family 2 |
| | | | | | | | | | | | | (facilitated glucose |
| | | | | | | | | | | | | transporter), member 13 |
| 219037_at | 1.30 | 0.0032 | 96.33 | 125.29 | 0.44 | 0.58 | 0.11 | 0.04 | 0.0044 | NM_016052 | CGI-115 | CGI-115 protein |
| 231270_at | 1.30 | 0.0096 | 36.64 | 47.45 | 0.17 | 0.22 | 0.11 | 0.06 | 0.0560 | BF111998 | CA13 | Carbonic anhydrase XIII |
| 230958_s_at | 1.29 | 0.0055 | 86.81 | 112.28 | 0.40 | 0.52 | 0.11 | 0.05 | 0.0227 | BE670797 | 230958_s_at | Transcribed locus |
| 205305_at | 1.29 | 0.0097 | 9.42 | 12.15 | 0.04 | 0.06 | 0.11 | 0.08 | 0.1519 | NM_004467 | FGL1 | Fibrinogen-like 1 |
| 243964_at | 1.29 | 0.0032 | 80.88 | 104.03 | 0.37 | 0.48 | 0.11 | 0.05 | 0.0230 | AI631201 | SHPRH | Transcribed locus |
| 1558517_s_at | 1.28 | 0.0096 | 22.44 | 28.81 | 0.10 | 0.13 | 0.11 | 0.05 | 0.0222 | CA773938 | LRRC8C | Leucine rich repeat |
| | | | | | | | | | | | | containing 8 family, |
| | | | | | | | | | | | | member C |
| 1561820_at | 1.28 | 0.0055 | 6.22 | 7.98 | 0.03 | 0.04 | 0.11 | 0.07 | 0.1238 | BQ337986 | SCN8A | Sodium channel, voltage |
| | | | | | | | | | | | | gated, type VIII, alpha |
| 209740_s_at | 1.27 | 0.0062 | 24.06 | 30.67 | 0.11 | 0.14 | 0.11 | 0.05 | 0.0334 | U03886 | PNPLA4 | Patatin-like phospholipase |
| | | | | | | | | | | | | domain containing 4 |
| 210203_at | 1.27 | 0.0057 | 18.04 | 22.95 | 0.08 | 0.11 | 0.10 | 0.06 | 0.0944 | R64001 | CNOT4 | CCR4-NOT transcription |
| | | | | | | | | | | | | complex, subunit 4 |
| 1555947_at | 1.27 | 0.0055 | 12.74 | 16.17 | 0.06 | 0.08 | 0.10 | 0.06 | 0.0945 | AU116818 | FAM120A | Chromosome 9 open |
| | | | | | | | | | | | | reading frame 10 |
| 210100_s_at | 1.27 | 0.0085 | 14.15 | 17.94 | 0.07 | 0.08 | 0.10 | 0.05 | 0.0425 | AF327657 | ABCA2 | ATP-binding cassette, sub- |
| | | | | | | | | | | | | family A (ABC1), member 2 |
| 1570259_at | 1.26 | 0.0067 | 35.45 | 44.51 | 0.16 | 0.21 | 0.10 | 0.05 | 0.0329 | BC015843 | LIMS1 | LIM and senescent cell |
| | | | | | | | | | | | | antigen-like domains 1 |
| 1568815_a_at | 1.25 | 0.0012 | 33.56 | 41.95 | 0.15 | 0.19 | 0.10 | 0.03 | 0.0040 | AA903184 | DDX50 | DEAD (Asp-Glu-Ala-Asp) |
| | | | | | | | | | | | | box polypeptide 50 |
| 213939_s_at | 1.24 | 0.0039 | 186.04 | 231.52 | 0.86 | 1.08 | 0.09 | 0.04 | 0.0110 | AI871641 | RUFY3 | RUN and FYVE domain |
| | | | | | | | | | | | | containing 3 |
| 234192_s_at | 1.24 | 0.0025 | 118.13 | 146.95 | 0.54 | 0.68 | 0.09 | 0.03 | 0.0021 | AK026487 | GKAP1 | G kinase anchoring protein 1 |
| 215919_s_at | 1.24 | 0.0061 | 36.66 | 45.58 | 0.17 | 0.21 | 0.09 | 0.04 | 0.0230 | BC000200 | MRPS11 | Homo sapiens, clone |
| | | | | | | | | | | | | IMAGE: 3352526, mRNA, |
| | | | | | | | | | | | | partial cds. |
| 229526_at | 1.24 | 0.0066 | 15.82 | 19.65 | 0.07 | 0.09 | 0.09 | 0.04 | 0.0271 | AI886656 | AQP11 | Aquaporin 11 |
| 226806_s_at | 1.24 | 0.0046 | 100.13 | 124.18 | 0.46 | 0.58 | 0.09 | 0.03 | 0.0074 | AW022607 | 226806_s_at | Full length insert cDNA |
| | | | | | | | | | | | | clone ZD69D05 |
| 205074_at | 1.23 | 0.0006 | 321.85 | 397.29 | 1.48 | 1.84 | 0.09 | 0.02 | 0.0002 | NM_003060 | SLC22A5 | Solute carrier family 22 |
| | | | | | | | | | | | | (organic cation transporter), |
| | | | | | | | | | | | | member 5 |
| 219343_at | 1.23 | 0.0081 | 198.86 | 245.35 | 0.92 | 1.14 | 0.09 | 0.03 | 0.0048 | NM_017913 | CDC37L1 | CDC37 cell division cycle |
| | | | | | | | | | | | | 37 homolog (S. cerevisiae)- |
| | | | | | | | | | | | | like 1 |
| 227256_at | 1.23 | 0.0048 | 67.37 | 82.84 | 0.31 | 0.38 | 0.09 | 0.03 | 0.0075 | BG289456 | USP31 | Ubiquitin specific peptidase |
| | | | | | | | | | | | | 31 |
| 1556382_a_at | 1.23 | 0.0070 | 55.76 | 68.55 | 0.26 | 0.32 | 0.09 | 0.04 | 0.0378 | AK091308 | NARG1 | NMDA receptor regulated 1 |
| 233748_x_at | 1.23 | 0.0021 | 67.81 | 83.24 | 0.31 | 0.39 | 0.09 | 0.03 | 0.0007 | AJ249976 | PRKAG2 | Potassium voltage-gated |
| | | | | | | | | | | | | channel, subfamily H (eag- |
| | | | | | | | | | | | | related), member 2 |
| 238640_at | 1.22 | 0.0040 | 32.67 | 40.00 | 0.15 | 0.19 | 0.09 | 0.04 | 0.0237 | AA878325 | 238640_at | oe61h04.s1 |
| | | | | | | | | | | | | NCI_CGAP_Lu5 Homo |
| | | | | | | | | | | | | sapiens cDNA clone |
| | | | | | | | | | | | | IMAGE: 1416151 3′, |
| | | | | | | | | | | | | mRNA sequence. |
| 217693_x_at | 1.22 | 0.0078 | 29.24 | 35.69 | 0.13 | 0.17 | 0.09 | 0.05 | 0.0851 | AW469555 | LOC388335 | similar to RIKEN cDNA |
| | | | | | | | | | | | | A730055C05 gene |
| 218772_x_at | 1.21 | 0.0046 | 87.21 | 105.92 | 0.40 | 0.49 | 0.08 | 0.03 | 0.0019 | NM_018112 | TMEM38B | Transmembrane protein |
| | | | | | | | | | | | | 38B |
| 226753_at | 1.20 | 0.0034 | 460.17 | 553.82 | 2.12 | 2.57 | 0.08 | 0.03 | 0.0064 | AW138704 | FAM76B | Family with sequence |
| | | | | | | | | | | | | similarity 76, member B |
| 212792_at | 1.20 | 0.0020 | 195.62 | 235.08 | 0.90 | 1.09 | 0.08 | 0.04 | 0.0606 | AB020684 | DPY19L1 | Dpy-19-like 1 (C. elegans) |
| 218292_s_at | 1.20 | 0.0016 | 72.73 | 86.95 | 0.34 | 0.40 | 0.08 | 0.03 | 0.0043 | NM_016203 | PRKAG2 | Potassium voltage-gated |
| | | | | | | | | | | | | channel, subfamily H (eag- |
| | | | | | | | | | | | | related), member 2 |
| 231913_s_at | 1.19 | 0.0014 | 81.37 | 96.78 | 0.37 | 0.45 | 0.08 | 0.02 | 0.0019 | X64643 | BRCC3 | BRCA1/BRCA2- |
| | | | | | | | | | | | | containing complex, |
| | | | | | | | | | | | | subunit 3 |
| 215549_x_at | 1.17 | 0.0085 | 59.73 | 70.13 | 0.28 | 0.33 | 0.07 | 0.03 | 0.0226 | AC005587 | WUGSC: H_DJ0988G15.1 | Homo sapiens PAC clone |
| | | | | | | | | | | | | RP5-988G15 from 6, |
| | | | | | | | | | | | | complete sequence. |
| 208757_at | 1.17 | 0.0076 | 55.27 | 64.87 | 0.25 | 0.30 | 0.07 | 0.04 | 0.0976 | BC001123 | TMED9 | Transmembrane emp24 |
| | | | | | | | | | | | | protein transport domain |
| | | | | | | | | | | | | containing 9 |
| 1557830_at | 1.17 | 0.0099 | 91.89 | 107.64 | 0.42 | 0.50 | 0.07 | 0.03 | 0.0143 | AW063658 | FLJ34077 | weakly similar to zinc |
| | | | | | | | | | | | | finger protein 195 |
| 225290_at | 1.16 | 0.0069 | 355.21 | 413.21 | 1.64 | 1.92 | 0.07 | 0.02 | 0.0036 | AV692425 | ETNK1 | Ethanolamine kinase 1 |
| 237749_at | 1.16 | 0.0026 | 7.48 | 8.70 | 0.03 | 0.04 | 0.07 | 0.06 | 0.2616 | AI027479 | TCF4 | Transcription factor 4 |
| 202127_at | 1.16 | 0.0058 | 483.37 | 561.26 | 2.23 | 2.61 | 0.06 | 0.02 | 0.0028 | AB011108 | PRPF4B | Homo sapiens mRNA for |
| | | | | | | | | | | | | KIAA0536 protein, partial |
| | | | | | | | | | | | | cds. |
| 1562391_at | 1.16 | 0.0085 | 48.58 | 56.25 | 0.22 | 0.26 | 0.06 | 0.04 | 0.1148 | AK091497 | B3GALNT2 | Beta-1,3-N- |
| | | | | | | | | | | | | acetylgalactosaminyltransferase 2 |
| 217983_s_at | 1.16 | 0.0011 | 2571.92 | 2976.17 | 11.85 | 13.82 | 0.06 | 0.02 | 0.0025 | NM_003730 | RNASET2 | Ribonuclease T2 |
| 201016_at | 1.16 | 0.0043 | 393.60 | 455.30 | 1.81 | 2.11 | 0.06 | 0.02 | 0.0052 | BE542684 | EIF1AX | Eukaryotic translation |
| | | | | | | | | | | | | initiation factor 1A, X- |
| | | | | | | | | | | | | linked |
| 202850_at | 1.15 | 0.0077 | 346.58 | 397.44 | 1.60 | 1.85 | 0.06 | 0.03 | 0.0270 | NM_002858 | ABCD3 | ATP-binding cassette, sub- |
| | | | | | | | | | | | | family D (ALD), member 3 |
| 203313_s_at | 1.15 | 0.0073 | 201.75 | 231.33 | 0.93 | 1.07 | 0.06 | 0.02 | 0.0097 | NM_003244 | TGIF | TGFB-induced factor |
| | | | | | | | | | | | | (TALE family homeobox) |
| 222673_x_at | 1.14 | 0.0023 | 502.50 | 572.98 | 2.32 | 2.66 | 0.06 | 0.02 | 0.0048 | AI582192 | FAM122B | Similar to hypothetical |
| | | | | | | | | | | | | protein MGC17347 |
| 213016_at | 1.14 | 0.0061 | 100.72 | 114.75 | 0.46 | 0.53 | 0.06 | 0.02 | 0.0067 | AA573805 | BBX | ARTC1 mRNA, complete |
| | | | | | | | | | | | | sequence |
| 212397_at | 1.14 | 0.0097 | 357.91 | 407.49 | 1.65 | 1.89 | 0.06 | 0.02 | 0.0149 | AL137751 | RDX | Homo sapiens mRNA; |
| | | | | | | | | | | | | cDNA DKFZp434I0812 |
| | | | | | | | | | | | | (from clone |
| | | | | | | | | | | | | DKFZp434I0812); partial |
| | | | | | | | | | | | | cds. |
| 218588_s_at | 1.14 | 0.0016 | 285.46 | 324.72 | 1.32 | 1.51 | 0.06 | 0.02 | 0.0012 | NM_018691 | C5orf3 | Chromosome 5 open |
| | | | | | | | | | | | | reading frame 3 |
| 211967_at | 1.14 | 0.0073 | 1496.46 | 1698.58 | 6.90 | 7.89 | 0.06 | 0.02 | 0.0057 | BG538627 | TMEM123 | Transmembrane protein |
| | | | | | | | | | | | | 123 |
| 223015_at | 1.12 | 0.0094 | 345.28 | 388.10 | 1.59 | 1.80 | 0.05 | 0.02 | 0.0121 | AF212241 | EIF2A | Eukaryotic translation |
| | | | | | | | | | | | | initiation factor 2A, 65 kDa |
| 213140_s_at | 1.12 | 0.0075 | 360.32 | 404.85 | 1.66 | 1.88 | 0.05 | 0.02 | 0.0135 | AB014593 | SS18L1 | Synovial sarcoma |
| | | | | | | | | | | | | translocation gene on |
| | | | | | | | | | | | | chromosome 18-like 1 |
| 221782_at | 1.12 | 0.0094 | 153.13 | 171.78 | 0.71 | 0.80 | 0.05 | 0.03 | 0.1180 | BG168666 | DNAJC10 | DnaJ (Hsp40) homolog, |
| | | | | | | | | | | | | subfamily C, member 10 |
| 221568_s_at | 1.12 | 0.0076 | 334.05 | 374.63 | 1.54 | 1.74 | 0.05 | 0.02 | 0.0259 | AF090900 | LIN7C | Lin-7 homolog C (C. elegans) |
| 213612_x_at | 1.12 | 0.0086 | 2230.73 | 2492.77 | 10.28 | 11.57 | 0.05 | 0.02 | 0.0087 | AI800419 | NBPF12 | Neuroblastoma breakpoint |
| | | | | | | | | | | | | family, member 12 |
| 1567222_x_at | 1.12 | 0.0027 | 106.13 | 118.58 | 0.49 | 0.55 | 0.05 | 0.03 | 0.0696 | D17207 | ELOVL5 | D17207 Kiseru Homo |
| | | | | | | | | | | | | sapiens cDNA clone |
| | | | | | | | | | | | | hmd3e08m3, mRNA |
| | | | | | | | | | | | | sequence. |
| 206900_x_at | 1.12 | 0.0048 | 160.48 | 179.03 | 0.74 | 0.83 | 0.05 | 0.02 | 0.0443 | NM_021047 | ZNF253 | Zinc finger protein 93 |
| 201319_at | 1.11 | 0.0031 | 969.96 | 1075.32 | 4.47 | 4.99 | 0.04 | 0.02 | 0.0233 | NM_006471 | MRCL3 | myosin regulatory light |
| | | | | | | | | | | | | chain MRCL3 |
| 200686_s_at | 1.11 | 0.0059 | 936.94 | 1038.66 | 4.32 | 4.82 | 0.04 | 0.02 | 0.0154 | NM_004768 | SFRS11 | Splicing factor, |
| | | | | | | | | | | | | arginine/serine-rich 11 |
| 206469_x_at | 1.11 | 0.0081 | 47.82 | 52.99 | 0.22 | 0.25 | 0.04 | 0.04 | 0.2604 | NM_012067 | AKR7A3 | Aldo-keto reductase family |
| | | | | | | | | | | | | 7, member A3 (aflatoxin |
| | | | | | | | | | | | | aldehyde reductase) |
| 223077_at | 1.10 | 0.0080 | 1147.18 | 1266.53 | 5.29 | 5.88 | 0.04 | 0.02 | 0.0073 | AW576360 | TMOD3 | Tropomodulin 3 |
| | | | | | | | | | | | | (ubiquitous) |
| 224565_at | 1.09 | 0.0084 | 1395.91 | 1525.92 | 6.43 | 7.09 | 0.04 | 0.02 | 0.0660 | BE675516 | TncRNA | trophoblast-derived |
| | | | | | | | | | | | | noncoding RNA |
| 208671_at | 1.09 | 0.0083 | 1296.31 | 1416.78 | 5.97 | 6.58 | 0.04 | 0.02 | 0.0140 | AF164794 | SERINC1 | Serine incorporator 1 |
| 213619_at | 1.09 | 0.0065 | 5014.22 | 5475.80 | 23.10 | 25.43 | 0.04 | 0.01 | 0.0074 | AV753392 | HNRPH1 | Heterogeneous nuclear |
| | | | | | | | | | | | | ribonucleoprotein H1 (H) |
| 204009_s_at | 1.08 | 0.0070 | 1745.06 | 1892.09 | 8.04 | 8.79 | 0.04 | 0.01 | 0.0044 | W80678 | KRAS | V-Ki-ras2 Kirsten rat |
| | | | | | | | | | | | | sarcoma viral oncogene |
| | | | | | | | | | | | | homolog |
| 207941_s_at | 1.07 | 0.0087 | 2806.53 | 3013.63 | 12.93 | 13.99 | 0.03 | 0.01 | 0.0195 | NM_004902 | RBM39 | RNA-binding region |
| | | | | | | | | | | | | (RNP1, RRM) containing 2 |
| 200640_at | 1.06 | 0.0053 | 4008.12 | 4240.19 | 18.47 | 19.69 | 0.02 | 0.01 | 0.0037 | NM_003406 | YWHAZ | Tyrosine 3- |
| | | | | | | | | | | | | monooxygenase/tryptophan |
| | | | | | | | | | | | | 5-monooxygenase |
| | | | | | | | | | | | | activation protein, zeta |
| | | | | | | | | | | | | polypeptide |
| 1553677_a_at | 1.02 | 0.0011 | 173.21 | 177.08 | 0.80 | 0.82 | 0.01 | 0.02 | 0.6582 | NM_152902 | TIPRL | TIP41, TOR signalling |
| | | | | | | | | | | | | pathway regulator-like (S. cerevisiae) |
| 209357_at | −1.07 | 0.0047 | 434.98 | 405.61 | 2.00 | 1.88 | −0.03 | 0.01 | 0.0223 | AF109161 | CITED2 | Cbp/p300-interacting |
| | | | | | | | | | | | | transactivator, with |
| | | | | | | | | | | | | Glu/Asp-rich carboxy- |
| | | | | | | | | | | | | terminal domain, 2 |
| 206180_x_at | −1.08 | 0.0009 | 684.71 | 636.30 | 3.15 | 2.95 | −0.03 | 0.01 | 0.0013 | NM_023931 | ZNF747 | Zinc finger protein 747 |
| 205022_s_at | −1.08 | 0.0062 | 1248.21 | 1159.57 | 5.75 | 5.38 | −0.03 | 0.01 | 0.0095 | NM_005197 | CHES1 | Checkpoint suppressor 1 |
| 200759_x_at | −1.08 | 0.0072 | 930.98 | 862.58 | 4.29 | 4.01 | −0.03 | 0.01 | 0.0072 | NM_003204 | NFE2L1 | Nuclear factor (erythroid- |
| | | | | | | | | | | | | derived 2)-like 1 |
| 217896_s_at | −1.10 | 0.0073 | 464.38 | 422.02 | 2.14 | 1.96 | −0.04 | 0.02 | 0.0123 | NM_024946 | NIP30 | NEFA-interacting nuclear |
| | | | | | | | | | | | | protein NIP30 |
| 205417_s_at | −1.10 | 0.0048 | 129.97 | 118.02 | 0.60 | 0.55 | −0.04 | 0.03 | 0.0936 | NM_004393 | DAG1 | Dystroglycan 1 |
| | | | | | | | | | | | | (dystrophin-associated |
| | | | | | | | | | | | | glycoprotein 1) |
| 211271_x_at | −1.10 | 0.0074 | 566.15 | 513.94 | 2.61 | 2.39 | −0.04 | 0.02 | 0.0093 | BC004383 | PTBP1 | Polypyrimidine tract |
| | | | | | | | | | | | | binding protein 1 |
| 225371_at | −1.10 | 0.0095 | 945.36 | 857.53 | 4.36 | 3.98 | −0.04 | 0.02 | 0.0096 | AI638714 | GLE1L | GLE1 RNA export |
| | | | | | | | | | | | | mediator-like (yeast) |
| 217650_x_at | −1.10 | 0.0056 | 620.36 | 562.49 | 2.86 | 2.61 | −0.04 | 0.02 | 0.0409 | AI088162 | ST3GAL2 | oz96b09.x1 |
| | | | | | | | | | | | | Soares_parathyroid_tumor_NbHPA |
| | | | | | | | | | | | | Homo sapiens |
| | | | | | | | | | | | | cDNA clone |
| | | | | | | | | | | | | IMAGE: 1683161 3′ similar |
| | | | | | | | | | | | | to contains Alu repetitive |
| | | | | | | | | | | | | element; mRNA sequence. |
| 1554597_at | −1.11 | 0.0030 | 957.55 | 865.44 | 4.41 | 4.02 | −0.04 | 0.01 | 0.0015 | BC016767.1 | LOC440426 | gb: BC016767.1 |
| | | | | | | | | | | | | /DB_XREF = gi: 16876988 |
| | | | | | | | | | | | | /TID = Hs2.30724.2 /CNT = 7 |
| | | | | | | | | | | | | /FEA = FLmRNA /TIER = FL |
| | | | | | | | | | | | | /STK = 1 /LL = 2967 |
| | | | | | | | | | | | | /UG_GENE = GTF2H3 |
| | | | | | | | | | | | | /UG = Hs.30724 |
| | | | | | | | | | | | | /DEF = Homo sapiens, |
| | | | | | | | | | | | | general transcription factor |
| | | | | | | | | | | | | IIH, polypeptide 3 (34 kD |
| | | | | | | | | | | | | subunit), clone MGC: 22720 |
| | | | | | | | | | | | | IMAGE: 4077665, mRNA, |
| | | | | | | | | | | | | complete cds. |
| | | | | | | | | | | | | /PROD = general |
| | | | | | | | | | | | | transcription factor IIH, |
| | | | | | | | | | | | | polypeptide 3(34 kD |
| | | | | | | | | | | | | subunit) |
| | | | | | | | | | | | | /FL = gb: BC016767.1 |
| 231393_x_at | −1.11 | 0.0022 | 283.26 | 255.97 | 1.31 | 1.19 | −0.04 | 0.02 | 0.0124 | AW237165 | ZBTB43 | Transcribed locus, strongly |
| | | | | | | | | | | | | similar to XP_528433.1 |
| | | | | | | | | | | | | PREDICTED: similar to |
| | | | | | | | | | | | | KIAA0414 [Pan |
| | | | | | | | | | | | | troglodytes] |
| 212260_at | −1.11 | 0.0050 | 452.13 | 407.98 | 2.08 | 1.89 | −0.04 | 0.02 | 0.0059 | AL045800 | TNRC15 | Trinucleotide repeat |
| | | | | | | | | | | | | containing 15 |
| 208815_x_at | −1.11 | 0.0065 | 722.38 | 649.68 | 3.33 | 3.02 | −0.05 | 0.02 | 0.0078 | AB023420 | HSPA4 | Heat shock 70 kDa protein 4 |
| 204362_at | −1.13 | 0.0029 | 2718.46 | 2395.77 | 12.53 | 11.12 | −0.05 | 0.02 | 0.0070 | NM_003930 | SCAP2 | Src family associated |
| | | | | | | | | | | | | phosphoprotein 2 |
| 219402_s_at | −1.13 | 0.0043 | 563.54 | 496.56 | 2.60 | 2.31 | −0.05 | 0.02 | 0.0039 | NM_024295 | DERL1 | Der1-like domain family, |
| | | | | | | | | | | | | member 1 |
| 203122_at | −1.15 | 0.0100 | 513.33 | 444.82 | 2.37 | 2.07 | −0.06 | 0.04 | 0.0839 | NM_016030 | TTC15 | Tetratricopeptide repeat |
| | | | | | | | | | | | | domain 15 |
| 214200_s_at | −1.16 | 0.0087 | 19.88 | 17.18 | 0.09 | 0.08 | −0.06 | 0.04 | 0.0865 | AI193744 | COL6A1 | Collagen, type VI, alpha 1 |
| 202401_s_at | −1.16 | 0.0059 | 418.49 | 359.42 | 1.93 | 1.67 | −0.07 | 0.02 | 0.0066 | NM_003131 | SRF | Serum response factor (c- |
| | | | | | | | | | | | | fos serum response |
| | | | | | | | | | | | | element-binding |
| | | | | | | | | | | | | transcription factor) |
| 1562979_at | −1.17 | 0.0078 | 19.85 | 16.99 | 0.09 | 0.08 | −0.07 | 0.04 | 0.1274 | BC037412 | COL5A3 | CDNA clone |
| | | | | | | | | | | | | IMAGE: 4939660 |
| 230631_s_at | −1.17 | 0.0092 | 572.65 | 488.68 | 2.64 | 2.27 | −0.07 | 0.03 | 0.0095 | AI202642 | IL10RB | Interleukin 10 receptor, |
| | | | | | | | | | | | | beta |
| 201583_s_at | −1.18 | 0.0062 | 378.33 | 321.01 | 1.74 | 1.49 | −0.07 | 0.03 | 0.0068 | NM_006363 | SEC23B | Sec23 homolog B (S. cerevisiae) |
| 213509_x_at | −1.18 | 0.0095 | 66.49 | 56.39 | 0.31 | 0.26 | −0.07 | 0.04 | 0.0681 | AW157619 | CES2 | Carboxylesterase 2 |
| | | | | | | | | | | | | (intestine, liver) |
| 230370_x_at | −1.18 | 0.0030 | 340.84 | 288.08 | 1.57 | 1.34 | −0.07 | 0.02 | 0.0029 | AI244335 | STYXL1 | Serine/threonine/tyrosine |
| | | | | | | | | | | | | interacting-like 1 |
| 218385_at | −1.19 | 0.0071 | 180.39 | 152.16 | 0.83 | 0.71 | −0.07 | 0.03 | 0.0050 | NM_018135 | MRPS18A | Mitochondrial ribosomal |
| | | | | | | | | | | | | protein S18A |
| 206222_at | −1.19 | 0.0099 | 1230.92 | 1037.38 | 5.67 | 4.82 | −0.07 | 0.03 | 0.0172 | NM_003841 | TNFRSF10C | Tumor necrosis factor |
| | | | | | | | | | | | | receptor superfamily, |
| | | | | | | | | | | | | member 10c, decoy without |
| | | | | | | | | | | | | an intracellular domain |
| 218879_s_at | −1.21 | 0.0001 | 161.83 | 134.27 | 0.75 | 0.62 | −0.08 | 0.02 | 0.0001 | NM_022764 | MTHFSD | Methenyltetrahydrofolate |
| | | | | | | | | | | | | synthetase domain |
| | | | | | | | | | | | | containing |
| 220753_s_at | −1.22 | 0.0053 | 95.21 | 78.00 | 0.44 | 0.36 | −0.09 | 0.05 | 0.0995 | NM_015974 | CRYL1 | Crystallin, lambda 1 |
| 226049_at | −1.23 | 0.0032 | 78.71 | 64.10 | 0.36 | 0.30 | −0.09 | 0.04 | 0.0229 | AI271420 | RAB6IP2 | RAB6 interacting protein 2 |
| 204340_at | −1.24 | 0.0071 | 126.84 | 102.39 | 0.58 | 0.48 | −0.09 | 0.04 | 0.0173 | NM_003492 | CXorf12 | Chromosome X open |
| | | | | | | | | | | | | reading frame 12 |
| 228015_s_at | −1.24 | 0.0095 | 52.43 | 42.31 | 0.24 | 0.20 | −0.09 | 0.05 | 0.0478 | BF115135 | TRIM8 | Tripartite motif-containing 8 |
| 212877_at | −1.24 | 0.0031 | 87.56 | 70.59 | 0.40 | 0.33 | −0.09 | 0.05 | 0.0395 | AA284075 | KNS2 | Kinesin 2 |
| 210920_x_at | −1.25 | 0.0082 | 35.27 | 28.26 | 0.16 | 0.13 | −0.10 | 0.05 | 0.0442 | NM_133457 | EMID2 | EMI domain containing 2 |
| 1558123_at | −1.25 | 0.0079 | 136.34 | 108.97 | 0.63 | 0.51 | −0.10 | 0.04 | 0.0097 | AK092709 | FLJ35390 | hypothetical protein |
| | | | | | | | | | | | | FLJ35390 |
| 204936_at | −1.26 | 0.0042 | 92.14 | 73.18 | 0.42 | 0.34 | −0.10 | 0.04 | 0.0254 | NM_004579 | MAP4K2 | Mitogen-activated protein |
| | | | | | | | | | | | | kinase kinase kinase kinase 2 |
| 202384_s_at | −1.26 | 0.0045 | 55.66 | 44.12 | 0.26 | 0.20 | −0.10 | 0.05 | 0.0651 | AW167713 | TCOF1 | Treacher Collins- |
| | | | | | | | | | | | | Franceschetti syndrome 1 |
| 1562106_at | −1.26 | 0.0040 | 10.79 | 8.55 | 0.05 | 0.04 | −0.10 | 0.06 | 0.0990 | BC039685 | PITRM1 | Pitrilysin metallopeptidase 1 |
| 203532_x_at | −1.27 | 0.0070 | 36.87 | 29.08 | 0.17 | 0.14 | −0.10 | 0.05 | 0.0204 | AF017061 | CUL5 | Cullin 5 |
| 227429_at | −1.27 | 0.0086 | 27.51 | 21.65 | 0.13 | 0.10 | −0.10 | 0.07 | 0.1095 | AI683694 | TSPAN4 | Tetraspanin 4 |
| 221953_s_at | −1.28 | 0.0084 | 81.09 | 63.46 | 0.37 | 0.29 | −0.11 | 0.05 | 0.0356 | W45551 | ITGB4BP | Integrin beta 4 binding |
| | | | | | | | | | | | | protein |
| 201755_at | −1.29 | 0.0066 | 32.12 | 25.00 | 0.15 | 0.12 | −0.11 | 0.05 | 0.0204 | NM_006739 | MCM5 | MCM5 minichromosome |
| | | | | | | | | | | | | maintenance deficient 5, |
| | | | | | | | | | | | | cell division cycle 46 (S. cerevisiae) |
| 243614_s_at | −1.29 | 0.0092 | 15.91 | 12.37 | 0.07 | 0.06 | −0.11 | 0.06 | 0.0567 | AW138125 | PRODH2 | Proline dehydrogenase |
| | | | | | | | | | | | | (oxidase) 2 |
| 236191_at | −1.29 | 0.0062 | 49.24 | 38.26 | 0.23 | 0.18 | −0.11 | 0.04 | 0.0069 | T81422 | CD38 | yd96d09.s1 Soares fetal |
| | | | | | | | | | | | | liver spleen 1NFLS Homo |
| | | | | | | | | | | | | sapiens cDNA clone |
| | | | | | | | | | | | | IMAGE: 116081 3′, mRNA |
| | | | | | | | | | | | | sequence. |
| 203730_s_at | −1.29 | 0.0093 | 63.79 | 49.44 | 0.29 | 0.23 | −0.11 | 0.05 | 0.0176 | BF196931 | ZFP95 | Zinc finger protein 95 |
| | | | | | | | | | | | | homolog (mouse) |
| 222037_at | −1.30 | 0.0047 | 139.84 | 107.23 | 0.64 | 0.50 | −0.12 | 0.06 | 0.0495 | AI859865 | MCM4 | MCM4 minichromosome |
| | | | | | | | | | | | | maintenance deficient 4 (S. cerevisiae) |
| 203921_at | −1.31 | 0.0038 | 316.54 | 242.20 | 1.46 | 1.12 | −0.12 | 0.04 | 0.0058 | NM_004267 | CHST2 | Carbohydrate (N- |
| | | | | | | | | | | | | acetylglucosamine-6-O) |
| | | | | | | | | | | | | sulfotransferase 2 |
| 207205_at | −1.31 | 0.0090 | 225.92 | 172.18 | 1.04 | 0.80 | −0.12 | 0.05 | 0.0261 | NM_001817 | CEACAM4 | Carcinoembryonic antigen- |
| | | | | | | | | | | | | related cell adhesion |
| | | | | | | | | | | | | molecule 4 |
| 230978_at | −1.31 | 0.0058 | 8.83 | 6.71 | 0.04 | 0.03 | −0.12 | 0.06 | 0.0639 | AI199850 | LOC401464 | Full-length cDNA clone |
| | | | | | | | | | | | | CS0DI043YL16 of |
| | | | | | | | | | | | | Placenta Cot 25-normalized |
| | | | | | | | | | | | | of Homo sapiens (human) |
| 218168_s_at | −1.33 | 0.0035 | 794.30 | 598.62 | 3.66 | 2.78 | −0.12 | 0.05 | 0.0061 | NM_020247 | CABC1 | Chaperone, ABC1 activity |
| | | | | | | | | | | | | of bc1 complex like (S. pombe) |
| 228645_at | −1.33 | 0.0011 | 86.63 | 65.08 | 0.40 | 0.30 | −0.12 | 0.04 | 0.0014 | AV762892 | SNORA78 | Small nucleolar RNA, |
| | | | | | | | | | | | | H/ACA box 78 |
| 237977_at | −1.34 | 0.0098 | 18.43 | 13.79 | 0.08 | 0.06 | −0.13 | 0.06 | 0.0498 | AI215189 | LOC440518 | similar to Golgi |
| | | | | | | | | | | | | autoantigen, golgin |
| | | | | | | | | | | | | subfamily a, 2 |
| 226704_at | −1.35 | 0.0065 | 7.94 | 5.89 | 0.04 | 0.03 | −0.13 | 0.07 | 0.0666 | R76659 | UBE2J2 | Ubiquitin-conjugating |
| | | | | | | | | | | | | enzyme E2, J2 (UBC6 |
| | | | | | | | | | | | | homolog, yeast) |
| 235083_at | −1.36 | 0.0073 | 6.63 | 4.87 | 0.03 | 0.02 | −0.13 | 0.09 | 0.1425 | AI699847 | FLJ38359 | tz11g09.x1 |
| | | | | | | | | | | | | NCI_CGAP_Ut1 Homo |
| | | | | | | | | | | | | sapiens cDNA clone |
| | | | | | | | | | | | | IMAGE: 2288320 3′ similar |
| | | | | | | | | | | | | to contains MSR1.t2 MSR1 |
| | | | | | | | | | | | | repetitive element; mRNA |
| | | | | | | | | | | | | sequence. |
| 224123_at | −1.36 | 0.0042 | 17.68 | 12.96 | 0.08 | 0.06 | −0.13 | 0.06 | 0.0149 | AL136837 | DKFZp434F142 | hypothetical protein |
| | | | | | | | | | | | | DKFZp434F142 |
| 221882_s_at | −1.36 | 0.0060 | 405.88 | 297.50 | 1.87 | 1.38 | −0.13 | 0.05 | 0.0042 | AI636233 | TMEM8 | Transmembrane protein 8 |
| | | | | | | | | | | | | (five membrane-spanning |
| | | | | | | | | | | | | domains) |
| 232550_at | −1.37 | 0.0037 | 7.27 | 5.31 | 0.03 | 0.02 | −0.14 | 0.09 | 0.1138 | AK001976 | PREB | Prolactin regulatory |
| | | | | | | | | | | | | element binding |
| 236595_at | −1.37 | 0.0086 | 157.07 | 114.47 | 0.72 | 0.53 | −0.14 | 0.06 | 0.0125 | AA776458 | MGC4677 | hypothetical protein |
| | | | | | | | | | | | | MGC4677 |
| 1562000_at | −1.37 | 0.0058 | 7.70 | 5.60 | 0.04 | 0.03 | −0.14 | 0.06 | 0.0288 | BC014643 | LOC400620 | hypothetical gene |
| | | | | | | | | | | | | supported by BC035399 |
| 1567252_at | −1.38 | 0.0072 | 10.34 | 7.48 | 0.05 | 0.03 | −0.14 | 0.09 | 0.1257 | X64983 | OR10D3P | Olfactory receptor, family |
| | | | | | | | | | | | | 10, subfamily D, member 3 |
| | | | | | | | | | | | | pseudogene |
| 228642_at | −1.41 | 0.0065 | 592.99 | 421.03 | 2.73 | 1.95 | −0.15 | 0.07 | 0.0257 | BF593636 | HOXA2 | Homo sapiens, clone |
| | | | | | | | | | | | | IMAGE: 5019307, mRNA |
| 224347_x_at | −1.41 | 0.0097 | 37.38 | 26.42 | 0.17 | 0.12 | −0.15 | 0.07 | 0.0359 | AF116687 | UBE2J2 | Homo sapiens PRO2121 |
| | | | | | | | | | | | | mRNA, complete cds. |
| 205623_at | −1.42 | 0.0079 | 12.02 | 8.48 | 0.06 | 0.04 | −0.15 | 0.07 | 0.0266 | NM_000691 | ALDH3A1 | Aldehyde dehydrogenase 3 |
| | | | | | | | | | | | | family, memberA1 |
| 1561923_a_at | −1.43 | 0.0086 | 10.09 | 7.06 | 0.05 | 0.03 | −0.16 | 0.07 | 0.0358 | AF147425 | SF3B14 | splicing factor 3B, 14 kDa |
| | | | | | | | | | | | | subunit |
| 211059_s_at | −1.44 | 0.0075 | 19.06 | 13.21 | 0.09 | 0.06 | −0.16 | 0.09 | 0.0599 | BC006381 | GOLGA2 | Golgi autoantigen, golgin |
| | | | | | | | | | | | | subfamily a, 2 |
| 234845_at | −1.45 | 0.0011 | 8.65 | 5.97 | 0.04 | 0.03 | −0.16 | 0.08 | 0.0386 | AL162045 | DKFZp761P0212 | hypothetical protein |
| | | | | | | | | | | | | DKFZp761P0212 |
| 213512_at | −1.45 | 0.0008 | 6.63 | 4.57 | 0.03 | 0.02 | −0.16 | 0.07 | 0.0137 | BF109941 | C14orf79 | Chromosome 14 open |
| | | | | | | | | | | | | reading frame 79 |
| 1566517_at | −1.46 | 0.0083 | 6.25 | 4.27 | 0.03 | 0.02 | −0.17 | 0.07 | 0.0136 | AL832405 | 1566517_at | Homo sapiens genomic |
| | | | | | | | | | | | | DNA; cDNA |
| | | | | | | | | | | | | DKFZp667E1713 (from |
| | | | | | | | | | | | | clone DKFZp667E1713). |
| 243029_at | −1.47 | 0.0014 | 11.04 | 7.51 | 0.05 | 0.03 | −0.17 | 0.08 | 0.0393 | AL533967 | KREMEN1 | AL533967 Homo sapiens |
| | | | | | | | | | | | | FETAL BRAIN Homo |
| | | | | | | | | | | | | sapiens cDNA clone |
| | | | | | | | | | | | | CS0DF003YP21 5-PRIME, |
| | | | | | | | | | | | | mRNA sequence. |
| 1557131_at | −1.49 | 0.0066 | 16.68 | 11.22 | 0.08 | 0.05 | −0.17 | 0.07 | 0.0153 | BQ287966 | LOC254100 | hypothetical protein |
| | | | | | | | | | | | | LOC254100 |
| 211734_s_at | −1.51 | 0.0017 | 905.80 | 599.35 | 4.17 | 2.78 | −0.18 | 0.05 | 0.0004 | BC005912 | FCER1A | Fc fragment of IgE, high |
| | | | | | | | | | | | | affinity I, receptor for; |
| | | | | | | | | | | | | alpha polypeptide |
| 214639_s_at | −1.52 | 0.0043 | 46.76 | 30.77 | 0.22 | 0.14 | −0.18 | 0.08 | 0.0173 | S79910 | HOXA1 | Homeobox A1 |
| 214296_x_at | −1.52 | 0.0012 | 11.88 | 7.80 | 0.05 | 0.04 | −0.18 | 0.09 | 0.0437 | AV721013 | C19orf36 | Chromosome 19 open |
| | | | | | | | | | | | | reading frame 36 |
| 214457_at | −1.52 | 0.0010 | 77.89 | 51.10 | 0.36 | 0.24 | −0.18 | 0.06 | 0.0023 | NM_006735 | HOXA2 | Homeobox A2 |
| 239647_at | −1.52 | 0.0077 | 233.18 | 152.97 | 1.07 | 0.71 | −0.18 | 0.09 | 0.0390 | AA677272 | CHST13 | Carbohydrate (chondroitin |
| | | | | | | | | | | | | 4) sulfotransferase 13 |
| 215458_s_at | −1.54 | 0.0007 | 10.33 | 6.69 | 0.05 | 0.03 | −0.19 | 0.09 | 0.0284 | AF199364 | SMURF1 | SMAD specific E3 |
| | | | | | | | | | | | | ubiquitin protein ligase 1 |
| 1553063_at | −1.56 | 0.0019 | 21.21 | 13.58 | 0.10 | 0.06 | −0.19 | 0.08 | 0.0122 | NM_080819 | GPR78 | G protein-coupled receptor |
| | | | | | | | | | | | | 78 |
| 1556655_s_at | −1.56 | 0.0052 | 13.99 | 8.94 | 0.06 | 0.04 | −0.19 | 0.07 | 0.0062 | AI860021 | na | wm22h08.x1 |
| | | | | | | | | | | | | NCI_CGAP_Ut4 Homo |
| | | | | | | | | | | | | sapiens cDNA clone |
| | | | | | | | | | | | | IMAGE: 2436735 3′ similar |
| | | | | | | | | | | | | to contains Alu repetitive |
| | | | | | | | | | | | | element; contains element |
| | | | | | | | | | | | | MER40 repetitive element; |
| | | | | | | | | | | | | mRNA sequence. |
| 220234_at | −1.59 | 0.0030 | 14.73 | 9.26 | 0.07 | 0.04 | −0.20 | 0.08 | 0.0088 | NM_004056 | CA8 | Carbonic anhydrase VIII |
| 211364_at | −1.59 | 0.0034 | 7.09 | 4.45 | 0.03 | 0.02 | −0.20 | 0.08 | 0.0095 | AF109294 | MTAP | Methylthioadenosine |
| | | | | | | | | | | | | phosphorylase |
| 1565617_at | −1.60 | 0.0029 | 9.94 | 6.23 | 0.05 | 0.03 | −0.20 | 0.08 | 0.0132 | AK097628 | STMN3 | CDNA FLJ40309 fis, clone |
| | | | | | | | | | | | | TESTI2029470 |
| 208211_s_at | −1.60 | 0.0076 | 7.92 | 4.94 | 0.04 | 0.02 | −0.21 | 0.10 | 0.0455 | U66559 | ALK | Anaplastic lymphoma |
| | | | | | | | | | | | | kinase (Ki-1) |
| 1559732_at | −1.61 | 0.0032 | 10.87 | 6.74 | 0.05 | 0.03 | −0.21 | 0.09 | 0.0208 | AK056624 | KCNH2 | Potassium voltage-gated |
| | | | | | | | | | | | | channel, subfamily H (eag- |
| | | | | | | | | | | | | related), member 2 |
| 226523_at | −1.62 | 0.0040 | 9.02 | 5.58 | 0.04 | 0.03 | −0.21 | 0.11 | 0.0444 | AI082237 | TAGLN | Transgelin |
| 204347_at | −1.64 | 0.0062 | 8.02 | 4.90 | 0.04 | 0.02 | −0.21 | 0.11 | 0.0416 | AI653169 | AK3L1 | Transcribed locus |
| 231305_at | −1.66 | 0.0053 | 14.51 | 8.75 | 0.07 | 0.04 | −0.22 | 0.08 | 0.0052 | AI820801 | LOC643982 | hypothetical protein |
| | | | | | | | | | | | | LOC643982 |
| 239530_at | −1.66 | 0.0065 | 10.93 | 6.58 | 0.05 | 0.03 | −0.22 | 0.10 | 0.0257 | BG171323 | ADD2 | Clone 23700 mRNA |
| | | | | | | | | | | | | sequence |
| 207950_s_at | −1.67 | 0.0060 | 16.37 | 9.77 | 0.08 | 0.05 | −0.22 | 0.10 | 0.0181 | NM_001149 | ANK3 | Ankyrin 3, node of Ranvier |
| | | | | | | | | | | | | (ankyrin G) |
| 213520_at | −1.71 | 0.0016 | 25.77 | 15.09 | 0.12 | 0.07 | −0.23 | 0.07 | 0.0003 | NM_004260 | RECQL4 | RecQ protein-like 4 |
| 91684_g_at | −1.71 | 0.0061 | 23.03 | 13.47 | 0.11 | 0.06 | −0.23 | 0.11 | 0.0251 | AI571298 | EXOSC4 | Exosome component 4 |
| 205624_at | −1.71 | 0.0016 | 175.65 | 102.53 | 0.81 | 0.48 | −0.23 | 0.08 | 0.0019 | NM_001870 | CPA3 | Carboxypeptidase A3 (mast |
| | | | | | | | | | | | | cell) |
| 209710_at | −1.72 | 0.0027 | 184.75 | 107.39 | 0.85 | 0.50 | −0.24 | 0.09 | 0.0057 | AL563460 | GATA2 | GATA binding protein 2 |
| 1569386_at | −1.74 | 0.0081 | 7.15 | 4.12 | 0.03 | 0.02 | −0.24 | 0.12 | 0.0382 | BC028053 | LOC645677 | similar to ciliary rootlet |
| | | | | | | | | | | | | coiled-coil, rootletin |
| 234034_at | −1.77 | 0.0045 | 8.15 | 4.61 | 0.04 | 0.02 | −0.25 | 0.13 | 0.0416 | AL137510 | KCNMB4 | Potassium large |
| | | | | | | | | | | | | conductance calcium- |
| | | | | | | | | | | | | activated channel, |
| | | | | | | | | | | | | subfamily M, beta member 4 |
| 228525_at | −1.80 | 0.0098 | 8.66 | 4.82 | 0.04 | 0.02 | −0.25 | 0.12 | 0.0334 | AL583533 | SLC7A10 | Transcribed locus |
| 206771_at | −1.87 | 0.0070 | 25.31 | 13.55 | 0.12 | 0.06 | −0.27 | 0.11 | 0.0069 | NM_006953 | UPK3A | Uroplakin 3A |
| 239970_at | −2.04 | 0.0048 | 7.37 | 3.61 | 0.03 | 0.02 | −0.31 | 0.16 | 0.0386 | AI088361 | 239970_at | Transcribed locus |
| 239048_at | −2.05 | 0.0005 | 7.59 | 3.71 | 0.03 | 0.02 | −0.31 | 0.11 | 0.0041 | BG427399 | KIF1B | Kinesin family member 1B |
| 208335_s_at | −2.05 | 0.0023 | 35.41 | 17.31 | 0.16 | 0.08 | −0.31 | 0.15 | 0.0253 | NM_002036 | DARC | Duffy blood group, |
| | | | | | | | | | | | | chemokine receptor |
| 214209_s_at | −2.05 | 0.0069 | 12.69 | 6.18 | 0.06 | 0.03 | −0.31 | 0.16 | 0.0551 | BE504895 | ABCB9 | ATP-binding cassette, sub- |
| | | | | | | | | | | | | family B (MDR/TAP), |
| | | | | | | | | | | | | member 9 |
| 207641_at | −2.06 | 0.0024 | 16.17 | 7.87 | 0.07 | 0.04 | −0.31 | 0.12 | 0.0054 | NM_012452 | TNFRSF13B | Tumor necrosis factor |
| | | | | | | | | | | | | receptor superfamily, |
| | | | | | | | | | | | | member 13B |
| 219050_s_at | −2.13 | 0.0100 | 8.97 | 4.21 | 0.04 | 0.02 | −0.33 | 0.15 | 0.0193 | NM_014205 | ZNHIT2 | Zinc finger, HIT type 2 |
| 1557165_s_at | −2.14 | 0.0065 | 15.01 | 7.01 | 0.07 | 0.03 | −0.33 | 0.17 | 0.0346 | BM141828 | KLHL18 | Kelch-like 18 (Drosophila) |
| 210517_s_at | −2.22 | 0.0015 | 44.26 | 19.94 | 0.20 | 0.09 | −0.35 | 0.09 | 0.0001 | AB003476 | AKAP12 | A kinase (PRKA) anchor |
| | | | | | | | | | | | | protein (gravin) 12 |
| 219669_at | −2.63 | 0.0025 | 427.21 | 162.51 | 1.97 | 0.75 | −0.42 | 0.15 | 0.0034 | NM_020406 | CD177 | gb: NM_020406.1 |
| | | | | | | | | | | | | /DEF = Homo sapiens |
| | | | | | | | | | | | | polycythemia rubra vera 1; |
| | | | | | | | | | | | | cell surface receptor |
| | | | | | | | | | | | | (PRV1), mRNA. |
| | | | | | | | | | | | | /FEA = mRNA /GEN = PRV1 |
| | | | | | | | | | | | | /PROD = polycythemia |
| | | | | | | | | | | | | rubra vera 1; cell |
| | | | | | | | | | | | | surfacereceptor |
| | | | | | | | | | | | | /DB_XREF = gi: 9966888 |
| | | | | | | | | | | | | /UG = Hs.232165 |
| | | | | | | | | | | | | polycythemia rubra vera 1; |
| | | | | | | | | | | | | cell surface receptor |
| | | | | | | | | | | | | /FL = gb: AF146747.1 |
| | | | | | | | | | | | | gb: NM_020406.1 |
| 223906_s_at | −2.98 | 0.0050 | 9.99 | 3.35 | 0.05 | 0.02 | −0.47 | 0.24 | 0.0418 | AY014285 | TEX101 | Testis expressed sequence |
| | | | | | | | | | | | | 101 |

TABLE 3
69 Genes for Preterm Delivery

| Affy | | | | | Control | Preterm | | |
|---|---|---|---|---|---|---|---|---|
| Probe | Fold | t-test | Control | Preterm | (norm) | (norm) | Log | Log |
| IDs | Change | P-value | Intensity | Intensity | Intensity | Intensity | (Ratio) | (Error) |
| 206778_at | 2.97 | 0.0013 | 5.84 | 17.36 | 0.03 | 0.08 | 0.47 | 0.15 |
| 211613_s_at | 2.23 | 0.0051 | 5.46 | 12.18 | 0.03 | 0.06 | 0.35 | 0.14 |
| 207891_s_at | 2.10 | 0.0018 | 3.78 | 7.96 | 0.02 | 0.04 | 0.32 | 0.11 |
| 237009_at | 2.10 | 0.0088 | 8.48 | 17.82 | 0.04 | 0.08 | 0.32 | 0.15 |
| 205138_s_at | 2.05 | 0.0045 | 4.21 | 8.62 | 0.02 | 0.04 | 0.31 | 0.14 |
| 231313_at | 2.04 | 0.0038 | 3.75 | 7.65 | 0.02 | 0.04 | 0.31 | 0.13 |
| 1557610_at | 2.01 | 0.0048 | 5.60 | 11.23 | 0.03 | 0.05 | 0.30 | 0.11 |
| 237606_at | 1.82 | 0.0078 | 4.98 | 9.04 | 0.02 | 0.04 | 0.26 | 0.09 |
| 240803_at | 1.79 | 0.0069 | 5.00 | 8.95 | 0.02 | 0.04 | 0.25 | 0.12 |
| 205717_x_at | 1.77 | 0.0019 | 4.85 | 8.61 | 0.02 | 0.04 | 0.25 | 0.10 |
| 238635_at | 1.76 | 0.0055 | 6.69 | 11.77 | 0.03 | 0.05 | 0.25 | 0.09 |
| 1566645_at | 1.76 | 0.0058 | 7.79 | 13.68 | 0.04 | 0.06 | 0.24 | 0.11 |
| 216965_x_at | 1.74 | 0.0065 | 5.71 | 9.96 | 0.03 | 0.05 | 0.24 | 0.12 |
| 217374_x_at | 1.74 | 0.0010 | 14.52 | 25.30 | 0.07 | 0.12 | 0.24 | 0.12 |
| 230996_at | 1.72 | 0.0059 | 6.46 | 11.09 | 0.03 | 0.05 | 0.23 | 0.12 |
| 238621_at | 1.67 | 0.0044 | 10.97 | 18.28 | 0.05 | 0.08 | 0.22 | 0.10 |
| 218948_at | 1.66 | 0.0024 | 20.65 | 34.26 | 0.10 | 0.16 | 0.22 | 0.12 |
| 1567035_at | 1.65 | 0.0037 | 6.04 | 9.94 | 0.03 | 0.05 | 0.22 | 0.11 |
| 233015_at | 1.63 | 0.0090 | 5.10 | 8.34 | 0.02 | 0.04 | 0.21 | 0.11 |
| 243947_s_at | 1.63 | 0.0100 | 9.02 | 14.74 | 0.04 | 0.07 | 0.21 | 0.11 |
| 220703_at | 1.63 | 0.0071 | 19.74 | 32.25 | 0.09 | 0.15 | 0.21 | 0.08 |
| 202733_at | 1.62 | 0.0027 | 5.12 | 8.27 | 0.02 | 0.04 | 0.21 | 0.13 |
| 234896_at | 1.61 | 0.0053 | 5.02 | 8.09 | 0.02 | 0.04 | 0.21 | 0.09 |
| 242146_at | 1.60 | 0.0049 | 62.32 | 99.88 | 0.29 | 0.46 | 0.20 | 0.07 |
| 226498_at | 1.59 | 0.0085 | 8.44 | 13.46 | 0.04 | 0.06 | 0.20 | 0.09 |
| 244638_at | 1.56 | 0.0066 | 9.72 | 15.21 | 0.04 | 0.07 | 0.19 | 0.07 |
| 242174_at | 1.56 | 0.0009 | 5.00 | 7.79 | 0.02 | 0.04 | 0.19 | 0.07 |
| 227701_at | 1.55 | 0.0016 | 167.67 | 260.66 | 0.77 | 1.21 | 0.19 | 0.06 |
| 1559491_at | 1.54 | 0.0048 | 6.59 | 10.13 | 0.03 | 0.05 | 0.19 | 0.11 |
| 222727_s_at | 1.54 | 0.0062 | 5.04 | 7.74 | 0.02 | 0.04 | 0.19 | 0.07 |
| 240114_s_at | 1.53 | 0.0003 | 5.24 | 7.99 | 0.02 | 0.04 | 0.18 | 0.07 |
| 240539_at | 1.51 | 0.0068 | 6.04 | 9.14 | 0.03 | 0.04 | 0.18 | 0.09 |
| 1556439_at | 1.51 | 0.0060 | 4.09 | 6.17 | 0.02 | 0.03 | 0.18 | 0.08 |
| 1570038_at | 1.50 | 0.0043 | 13.56 | 20.30 | 0.06 | 0.09 | 0.18 | 0.09 |
| 211734_s_at | −1.51 | 0.0017 | 905.80 | 599.35 | 4.17 | 2.78 | −0.18 | 0.05 |
| 214639_s_at | −1.52 | 0.0043 | 46.76 | 30.77 | 0.22 | 0.14 | −0.18 | 0.08 |
| 214296_x_at | −1.52 | 0.0012 | 11.88 | 7.80 | 0.05 | 0.04 | −0.18 | 0.09 |
| 214457_at | −1.52 | 0.0010 | 77.89 | 51.10 | 0.36 | 0.24 | −0.18 | 0.06 |
| 239647_at | −1.52 | 0.0077 | 233.18 | 152.97 | 1.07 | 0.71 | −0.18 | 0.09 |
| 215458_s_at | −1.54 | 0.0007 | 10.33 | 6.69 | 0.05 | 0.03 | −0.19 | 0.09 |
| 1553063_at | −1.56 | 0.0019 | 21.21 | 13.58 | 0.10 | 0.06 | −0.19 | 0.08 |
| 1556655_s_at | −1.56 | 0.0052 | 13.99 | 8.94 | 0.06 | 0.04 | −0.19 | 0.07 |
| 220234_at | −1.59 | 0.0030 | 14.73 | 9.26 | 0.07 | 0.04 | −0.20 | 0.08 |
| 211364_at | −1.59 | 0.0034 | 7.09 | 4.45 | 0.03 | 0.02 | −0.20 | 0.08 |
| 1565617_at | −1.60 | 0.0029 | 9.94 | 6.23 | 0.05 | 0.03 | −0.20 | 0.08 |
| 208211_s_at | −1.60 | 0.0076 | 7.92 | 4.94 | 0.04 | 0.02 | −0.21 | 0.10 |
| 1559732_at | −1.61 | 0.0032 | 10.87 | 6.74 | 0.05 | 0.03 | −0.21 | 0.09 |
| 226523_at | −1.62 | 0.0040 | 9.02 | 5.58 | 0.04 | 0.03 | −0.21 | 0.11 |
| 204347_at | −1.64 | 0.0062 | 8.02 | 4.90 | 0.04 | 0.02 | −0.21 | 0.11 |
| 231305_at | −1.66 | 0.0053 | 14.51 | 8.75 | 0.07 | 0.04 | −0.22 | 0.08 |
| 239530_at | −1.66 | 0.0065 | 10.93 | 6.58 | 0.05 | 0.03 | −0.22 | 0.10 |
| 207950_s_at | −1.67 | 0.0060 | 16.37 | 9.77 | 0.08 | 0.05 | −0.22 | 0.10 |
| 213520_at | −1.71 | 0.0016 | 25.77 | 15.09 | 0.12 | 0.07 | −0.23 | 0.07 |
| 91684_g_at | −1.71 | 0.0061 | 23.03 | 13.47 | 0.11 | 0.06 | −0.23 | 0.11 |
| 205624_at | −1.71 | 0.0016 | 175.65 | 102.53 | 0.81 | 0.48 | −0.23 | 0.08 |
| 209710_at | −1.72 | 0.0027 | 184.75 | 107.39 | 0.85 | 0.50 | −0.24 | 0.09 |
| 1569386_at | −1.74 | 0.0081 | 7.15 | 4.12 | 0.03 | 0.02 | −0.24 | 0.12 |
| 234034_at | −1.77 | 0.0045 | 8.15 | 4.61 | 0.04 | 0.02 | −0.25 | 0.13 |
| 228525_at | −1.80 | 0.0098 | 8.66 | 4.82 | 0.04 | 0.02 | −0.25 | 0.12 |
| 206771_at | −1.87 | 0.0070 | 25.31 | 13.55 | 0.12 | 0.06 | −0.27 | 0.11 |
| 239970_at | −2.04 | 0.0048 | 7.37 | 3.61 | 0.03 | 0.02 | −0.31 | 0.16 |
| 239048_at | −2.05 | 0.0005 | 7.59 | 3.71 | 0.03 | 0.02 | −0.31 | 0.11 |
| 208335_s_at | −2.05 | 0.0023 | 35.41 | 17.31 | 0.16 | 0.08 | −0.31 | 0.15 |
| 214209_s_at | −2.05 | 0.0069 | 12.69 | 6.18 | 0.06 | 0.03 | −0.31 | 0.16 |
| 207641_at | −2.06 | 0.0024 | 16.17 | 7.87 | 0.07 | 0.04 | −0.31 | 0.12 |
| 219050_s_at | −2.13 | 0.0100 | 8.97 | 4.21 | 0.04 | 0.02 | −0.33 | 0.15 |
| 1557165_s_at | −2.14 | 0.0065 | 15.01 | 7.01 | 0.07 | 0.03 | −0.33 | 0.17 |
| 210517_s_at | −2.22 | 0.0015 | 44.26 | 19.94 | 0.20 | 0.09 | −0.35 | 0.09 |
| 219669_at | −2.63 | 0.0025 | 427.21 | 162.51 | 1.97 | 0.75 | −0.42 | 0.15 |
| 223906_s_at | −2.98 | 0.0050 | 9.99 | 3.35 | 0.05 | 0.02 | −0.47 | 0.24 |

| Affy | | | | |
|---|---|---|---|---|
| Probe | P-value | | Sequence | |
| IDs | Resolver | Accession # | Name | Sequence Description |
| 206778_at | 0.0016 | NM_000496 | CRYBB2 | Crystallin, beta B2 |
| 211613_s_at | 0.0111 | U79250 | GPD2 | Glycerol-3-phosphate |
| | | | | dehydrogenase 2 |
| | | | | (mitochondrial) |
| 207891_s_at | 0.0035 | NM_017518 | UIP1 | 26S proteasome-associated |
| | | | | UCH interacting protein 1 |
| 237009_at | 0.0311 | BF439675 | CD69 | CD69 molecule |
| 205138_s_at | 0.0187 | AW418882 | UST | Uronyl-2-sulfotransferase |
| 231313_at | 0.0160 | AW134984 | LRRC8B | Leucine rich repeat |
| | | | | containing 8 family, |
| | | | | member B |
| 1557610_at | 0.0051 | AI003930 | PITRM1 | Pitrilysin metallopeptidase 1 |
| 237606_at | 0.0056 | AI022073 | CD53 | CD53 molecule |
| 240803_at | 0.0400 | AW450626 | C1orf131 | Chromosome 1 open |
| | | | | reading frame 131 |
| 205717_x_at | 0.0138 | NM_002588 | PCDHGC3 | Protocadherin gamma |
| | | | | subfamily C, 3 |
| 238635_at | 0.0099 | W72333 | FLJ21657 | hypothetical protein |
| | | | | FLJ21657 |
| 1566645_at | 0.0256 | AL050106 | NHEJ1 | Nonhomologous end- |
| | | | | joining factor 1 |
| 216965_x_at | 0.0445 | AL139377 | SPG20 | Human DNA sequence |
| | | | | from clone RP11-251J8 on |
| | | | | chromosome 13 Contains 2 |
| | | | | novel genes, the KIAA0610 |
| | | | | gene and a CpG island, |
| | | | | complete sequence. |
| 217374_x_at | 0.0374 | AC006033 | STARD3NL | Homo sapiens BAC clone |
| | | | | RP11-121A8 from 7, |
| | | | | complete sequence. |
| 230996_at | 0.0428 | AW024499 | LOC339929 | hypothetical protein |
| | | | | LOC339929 |
| 238621_at | 0.0205 | R67695 | FMN1 | Formin 1 |
| 218948_at | 0.0491 | AL136679 | QRSL1 | Glutaminyl-tRNA synthase |
| | | | | (glutamine-hydrolyzing)- |
| | | | | like 1 |
| 1567035_at | 0.0484 | U63828 | C20orf181 | Chromosome 20 open |
| | | | | reading frame 181 |
| 233015_at | 0.0483 | AA732240 | MBNL1 | Muscleblind-like |
| | | | | (Drosophila) |
| 243947_s_at | 0.0564 | AW300612 | 243947_s_at | Transcribed locus |
| 220703_at | 0.0068 | NM_018470 | C10orf110 | Chromosome 10 open |
| | | | | reading frame 110 |
| 202733_at | 0.0901 | NM_004199 | P4HA2 | Procollagen-proline, 2- |
| | | | | oxoglutarate 4-dioxygenase |
| | | | | (proline 4-hydroxylase), |
| | | | | alpha polypeptide II |
| 234896_at | 0.0252 | AJ012680 | C1orf5 | Homo sapiens gene |
| | | | | encoding hypothetical |
| | | | | protein with HTH motif. |
| 242146_at | 0.0056 | AA872471 | SNRPA1 | Small nuclear |
| | | | | ribonucleoprotein |
| | | | | polypeptide A′ |
| 226498_at | 0.0213 | AA149648 | FLT1 | Fms-related tyrosine kinase |
| | | | | 1 (vascular endothelial |
| | | | | growth factor/vascular |
| | | | | permeability factor |
| | | | | receptor) |
| 244638_at | 0.0040 | AW954477 | SUCLG1 | Succinate-CoA ligase, |
| | | | | GDP-forming, alpha |
| | | | | subunit |
| 242174_at | 0.0035 | AI732542 | ZBTB10 | ni36g03.x5 |
| | | | | NCI_CGAP_Lu1 Homo |
| | | | | sapiens cDNA clone |
| | | | | IMAGE: 978964 3′ similar |
| | | | | to contains Alu repetitive |
| | | | | element; contains element |
| | | | | TAR1 TAR1 repetitive |
| | | | | element; mRNA sequence. |
| 227701_at | 0.0020 | AK024739 | C10orf118 | Chromosome 10 open |
| | | | | reading frame 118 |
| 1559491_at | 0.0682 | AL390180 | TNRC17 | MRNA; cDNA |
| | | | | DKFZp761L149 (from |
| | | | | clone DKFZp761L149) |
| 222727_s_at | 0.0121 | AI339568 | SLC24A6 | Solute carrier family 24 |
| | | | | (sodium/potassium/calcium |
| | | | | exchanger), member 6 |
| 240114_s_at | 0.0065 | AI927971 | MGC13034 | hypothetical protein |
| | | | | MGC13034 |
| 240539_at | 0.0367 | AI684551 | AUTS2 | Autism susceptibility |
| | | | | candidate 2 |
| 1556439_at | 0.0218 | AL832163 | LOC441376 | AARD protein |
| 1570038_at | 0.0583 | BC009008 | ZNF595 | Zinc finger protein 595 |
| 211734_s_at | 0.0004 | BC005912 | FCER1A | Fc fragment of IgE, high |
| | | | | affinity I, receptor for; |
| | | | | alpha polypeptide |
| 214639_s_at | 0.0173 | S79910 | HOXA1 | Homeobox A1 |
| 214296_x_at | 0.0437 | AV721013 | C19orf36 | Chromosome 19 open |
| | | | | reading frame 36 |
| 214457_at | 0.0023 | NM_006735 | HOXA2 | Homeobox A2 |
| 239647_at | 0.0390 | AA677272 | CHST13 | Carbohydrate (chondroitin |
| | | | | 4) sulfotransferase 13 |
| 215458_s_at | 0.0284 | AF199364 | SMURF1 | SMAD specific E3 |
| | | | | ubiquitin protein ligase 1 |
| 1553063_at | 0.0122 | NM_080819 | GPR78 | G protein-coupled receptor |
| | | | | 78 |
| 1556655_s_at | 0.0062 | AI860021 | na | wm22h08.x1 |
| | | | | NCI_CGAP_Ut4 Homo |
| | | | | sapiens cDNA clone |
| | | | | IMAGE: 2436735 3′ similar |
| | | | | to contains Alu repetitive |
| | | | | element; contains element |
| | | | | MER40 repetitive element; |
| | | | | mRNA sequence. |
| 220234_at | 0.0088 | NM_004056 | CA8 | Carbonic anhydrase VIII |
| 211364_at | 0.0095 | AF109294 | MTAP | Methylthioadenosine |
| | | | | phosphorylase |
| 1565617_at | 0.0132 | AK097628 | STMN3 | CDNA FLJ40309 fis, clone |
| | | | | TESTI2029470 |
| 208211_s_at | 0.0455 | U66559 | ALK | Anaplastic lymphoma |
| | | | | kinase (Ki-1) |
| 1559732_at | 0.0208 | AK056624 | KCNH2 | Potassium voltage-gated |
| | | | | channel, subfamily H (eag- |
| | | | | related), member 2 |
| 226523_at | 0.0444 | AI082237 | TAGLN | Transgelin |
| 204347_at | 0.0416 | AI653169 | AK3L1 | Transcribed locus |
| 231305_at | 0.0052 | AI820801 | LOC643982 | hypothetical protein |
| | | | | LOC643982 |
| 239530_at | 0.0257 | BG171323 | ADD2 | Clone 23700 mRNA |
| | | | | sequence |
| 207950_s_at | 0.0181 | NM_001149 | ANK3 | Ankyrin 3, node of Ranvier |
| | | | | (ankyrin G) |
| 213520_at | 0.0003 | NM_004260 | RECQL4 | RecQ protein-like 4 |
| 91684_g_at | 0.0251 | AI571298 | EXOSC4 | Exosome component 4 |
| 205624_at | 0.0019 | NM_001870 | CPA3 | Carboxypeptidase A3 (mast |
| | | | | cell) |
| 209710_at | 0.0057 | AL563460 | GATA2 | GATA binding protein 2 |
| 1569386_at | 0.0382 | BC028053 | LOC645677 | similar to ciliary rootlet |
| | | | | coiled-coil, rootletin |
| 234034_at | 0.0416 | AL137510 | KCNMB4 | Potassium large |
| | | | | conductance calcium- |
| | | | | activated channel, |
| | | | | subfamily M, beta member 4 |
| 228525_at | 0.0334 | AL583533 | SLC7A10 | Transcribed locus |
| 206771_at | 0.0069 | NM_006953 | UPK3A | Uroplakin 3A |
| 239970_at | 0.0386 | AI088361 | 239970_at | Transcribed locus |
| 239048_at | 0.0041 | BG427399 | KIF1B | Kinesin family member 1B |
| 208335_s_at | 0.0253 | NM_002036 | DARC | Duffy blood group, |
| | | | | chemokine receptor |
| 214209_s_at | 0.0551 | BE504895 | ABCB9 | ATP-binding cassette, sub- |
| | | | | family B (MDR/TAP), |
| | | | | member 9 |
| 207641_at | 0.0054 | NM_012452 | TNFRSF13B | Tumor necrosis factor |
| | | | | receptor superfamily, |
| | | | | member 13B |
| 219050_s_at | 0.0193 | NM_014205 | ZNHIT2 | Zinc finger, HIT type 2 |
| 1557165_s_at | 0.0346 | BM141828 | KLHL18 | Kelch-like 18 (Drosophila) |
| 210517_s_at | 0.0001 | AB003476 | AKAP12 | A kinase (PRKA) anchor |
| | | | | protein (gravin) 12 |
| 219669_at | 0.0034 | NM_020406 | CD177 | gb: NM_020406.1 |
| | | | | /DEF = Homo sapiens |
| | | | | polycythemia rubra vera 1; |
| | | | | cell surface receptor |
| | | | | (PRV1), mRNA. |
| | | | | /FEA = mRNA /GEN = PRV1 |
| | | | | /PROD = polycythemia |
| | | | | rubra vera 1; cell |
| | | | | surfacereceptor |
| | | | | /DB_XREF = gi: 9966888 |
| | | | | /UG = Hs.232165 |
| | | | | polycythemia rubra vera 1; |
| | | | | cell surface receptor |
| | | | | /FL = gb: AF146747.1 |
| | | | | gb: NM_020406.1 |
| 223906_s_at | 0.0418 | AY014285 | TEX101 | Testis expressed sequence |
| | | | | 101 |

TABLE 4
27 Genes for Preterm Delivery

| Affy | | t-test | | | | Control | Preterm | |
|---|---|---|---|---|---|---|---|---|
| Probe | Fold | P- | T- | Control | Preterm | (norm) | (norm) | Log |
| IDs | Change | value | statistic | Intensity | Intensity | Intensity | Intensity | (Ratio) |
| 237695_at | 8.18 | 0.0151 | −2.75 | 1.56 | 12.73 | 0.01 | 0.06 | 0.91 |
| 1566548_at | 5.24 | 0.0407 | −2.15 | 2.43 | 12.72 | 0.01 | 0.06 | 0.72 |
| 232897_at | 4.08 | 0.0494 | −2.14 | 3.74 | 15.25 | 0.02 | 0.07 | 0.61 |
| 206778_at | 2.97 | 0.0013 | −3.79 | 5.84 | 17.36 | 0.03 | 0.08 | 0.47 |
| 211613_s_at | 2.23 | 0.0051 | −3.06 | 5.46 | 12.18 | 0.03 | 0.06 | 0.35 |
| 206777_s_at | 2.20 | 0.0267 | −2.47 | 60.29 | 132.88 | 0.28 | 0.62 | 0.34 |
| 1566889_at | 2.19 | 0.0176 | −2.60 | 14.56 | 31.82 | 0.07 | 0.15 | 0.34 |
| 206856_at | 2.16 | 0.0234 | −2.47 | 5.33 | 11.55 | 0.02 | 0.05 | 0.34 |
| 207891_s_at | 2.10 | 0.0018 | −3.46 | 3.78 | 7.96 | 0.02 | 0.04 | 0.32 |
| 237009_at | 2.10 | 0.0088 | −2.82 | 8.48 | 17.82 | 0.04 | 0.08 | 0.32 |
| 205138_s_at | 2.05 | 0.0045 | −3.11 | 4.21 | 8.62 | 0.02 | 0.04 | 0.31 |
| 231313_at | 2.04 | 0.0038 | −3.25 | 3.75 | 7.65 | 0.02 | 0.04 | 0.31 |
| 1557610_at | 2.01 | 0.0048 | −3.11 | 5.60 | 11.23 | 0.03 | 0.05 | 0.30 |
| 218345_at | −2.01 | 0.0269 | 2.34 | 572.00 | 284.96 | 2.64 | 1.32 | −0.30 |
| 239970_at | −2.04 | 0.0048 | 3.08 | 7.37 | 3.61 | 0.03 | 0.02 | −0.31 |
| 239048_at | −2.05 | 0.0005 | 3.96 | 7.59 | 3.71 | 0.03 | 0.02 | −0.31 |
| 208335_s_at | −2.05 | 0.0023 | 3.41 | 35.41 | 17.31 | 0.16 | 0.08 | −0.31 |
| 214209_s_at | −2.05 | 0.0069 | 2.97 | 12.69 | 6.18 | 0.06 | 0.03 | −0.31 |
| 207641_at | −2.06 | 0.0024 | 3.34 | 16.17 | 7.87 | 0.07 | 0.04 | −0.31 |
| 224753_at | −2.06 | 0.0377 | 2.24 | 19.63 | 9.51 | 0.09 | 0.04 | −0.31 |
| 219050_s_at | −2.13 | 0.0100 | 2.77 | 8.97 | 4.21 | 0.04 | 0.02 | −0.33 |
| 1557165_s_at | −2.14 | 0.0065 | 2.94 | 15.01 | 7.01 | 0.07 | 0.03 | −0.33 |
| 210517_s_at | −2.22 | 0.0015 | 3.52 | 44.26 | 19.94 | 0.20 | 0.09 | −0.35 |
| 203559_s_at | −2.50 | 0.0305 | 2.28 | 54.30 | 21.68 | 0.25 | 0.10 | −0.40 |
| 219669_at | −2.63 | 0.0025 | 3.33 | 427.21 | 162.51 | 1.97 | 0.75 | −0.42 |
| 223906_s_at | −2.98 | 0.0050 | 3.06 | 9.99 | 3.35 | 0.05 | 0.02 | −0.47 |
| 219082_at | −4.01 | 0.0183 | 2.51 | 8.89 | 2.22 | 0.04 | 0.01 | −0.60 |

| Affy | | | | | |
|---|---|---|---|---|---|
| Probe | Log | P-value | | | |
| IDs | (Error) | Resolver | Accession # | Gene Name | Sequence Description |
| 237695_at | 0.53 | 0.0426 | BF197664 | LOC442421 | similar to prostaglandin E |
| | | | | | receptor 4, subtype EP4; |
| | | | | | PGE receptor, EP4 |
| | | | | | subtype; prostaglandin E2 |
| | | | | | receptor |
| 1566548_at | 0.14 | 0.0000 | AL049918 | DHRSX | Transcribed locus |
| 232897_at | 0.30 | 0.0342 | AK000451 | FLJ20444 | hypothetical protein |
| | | | | | FLJ20444 |
| 206778_at | 0.15 | 0.0016 | NM_000496 | CRYBB2 | Homo sapiens crystallin |
| | | | | | beta B2 (CRYBB2) |
| | | | | | mRNA. |
| 211613_s_at | 0.14 | 0.0111 | U79250 | GPD2 | Glycerol-3-phosphate |
| | | | | | dehydrogenase 2 |
| | | | | | (mitochondrial) |
| 206777_s_at | 0.12 | 0.0066 | NM_000496 | CRYBB2 | Homo sapiens crystallin |
| | | | | | beta B2 (CRYBB2) |
| | | | | | mRNA. |
| 1566889_at | 0.13 | 0.0118 | BC037847 | THADA | Thyroid adenoma |
| | | | | | associated |
| 206856_at | 0.14 | 0.0184 | NM_006840 | LILRB5 | Leukocyte |
| | | | | | immunoglobulin-like |
| | | | | | receptor, subfamily B |
| | | | | | (with TM and ITIM |
| | | | | | domains), member 2 |
| 207891_s_at | 0.11 | 0.0035 | NM_017518 | UIP1 | 26S proteasome-associated |
| | | | | | UCH interacting protein 1 |
| 237009_at | 0.15 | 0.0311 | BF439675 | CD69 | CD69 molecule |
| 205138_s_at | 0.14 | 0.0187 | AW418882 | UST | Uronyl-2-sulfotransferase |
| 231313_at | 0.13 | 0.0160 | AW134984 | LRRC8B | Leucine rich repeat |
| | | | | | containing 8 family, |
| | | | | | member B |
| 1557610_at | 0.11 | 0.0051 | AI003930 | PITRM1 | Pitrilysin metallopeptidase 1 |
| 218345_at | 0.14 | 0.0230 | NM_018487 | TMEM176A | Homo sapiens |
| | | | | | transmembrane protein |
| | | | | | 176A (TMEM176A) |
| | | | | | mRNA. |
| 239970_at | 0.16 | 0.0386 | AI088361 | 239970_at | Transcribed locus |
| 239048_at | 0.11 | 0.0041 | BG427399 | KIF1B | Transcribed locus |
| 208335_s_at | 0.15 | 0.0253 | NM_002036 | DARC | Homo sapiens Duffy blood |
| | | | | | group chemokine receptor |
| | | | | | (DARC) mRNA. |
| 214209_s_at | 0.16 | 0.0551 | BE504895 | ABCB9 | ATP-binding cassette, sub- |
| | | | | | family B (MDR/TAP), |
| | | | | | member 9 |
| 207641_at | 0.12 | 0.0054 | NM_012452 | TNFRSF13B | Homo sapiens tumor |
| | | | | | necrosis factor receptor |
| | | | | | superfamily member 13B |
| 224753_at | 0.17 | 0.0666 | BE614410 | CDCA5 | Cell division cycle |
| | | | | | associated 5 |
| 219050_s_at | 0.15 | 0.0193 | NM_014205 | ZNHIT2 | Homo sapiens zinc finger |
| | | | | | HIT type 2 (ZNHIT2) |
| | | | | | mRNA. |
| 1557165_s_at | 0.17 | 0.0346 | BM141828 | KLHL18 | Kelch-like 18 (Drosophila) |
| 210517_s_at | 0.09 | 0.0001 | AB003476 | AKAP12 | A kinase (PRKA) anchor |
| | | | | | protein (gravin) 12 |
| 203559_s_at | 0.22 | 0.0569 | NM_001091 | ABP1 | Homo sapiens amiloride |
| | | | | | binding protein 1 (amine |
| | | | | | oxidase |
| 219669_at | 0.15 | 0.0034 | NM_020406 | CD177 | Homo sapiens CD177 |
| | | | | | molecule (CD177) mRNA. |
| 223906_s_at | 0.24 | 0.0418 | AY014285 | TEX101 | Testis expressed sequence |
| | | | | | 101 |
| 219082_at | 0.30 | 0.0104 | NM_015944 | AMDHD2 | Homo sapiens |
| | | | | | amidohydrolase domain |
| | | | | | containing 2 (AMDHD2) |
| | | | | | mRNA. |

In one embodiment, the expression levels of a plurality of genes in the multimarker classifier from a plurality of women who delivered at term, and the expression levels of a plurality of genes in the multimarker classifier from a plurality of women who delivered preterm, are determined. For example, a representative data set of samples from a plurality of women who delivered at term and from a plurality of women who delivered preterm is collected. For example, samples from subjects meeting the definition and phenotypic sub-classification of sPTD based on criteria advocated by the PREBIC Genetics Working Group can be taken. For example, estimated date of conception (EDC) can be used to define preterm deliveries. EDC can be assessed using maternal report of last menstrual period (LMP) combined with ultrasound at ≦20 weeks gestation. If both LMP and ultrasound dating are available and the two agree within 14 days, the former can be used to assign gestational age. If the two differ by more than 14 days, ultrasound date can be used. Samples from term controls are also be taken.

In one embodiment, to minimize potential confounding by maternal race/ethnicity and to optimize statistical power of the classifier, analyses can be restricted to particular maternal races or ethnicities. Identical exclusion/selection and frequency matching criteria can be used to select participants for independent validation analyses.

Specimens for analysis for the multi-marker classifier can be selected using, for example, a nested case-control study design. For example, all sPTD cases in the study population are identified. VPTD cases and a balanced random sample of moderate cases to achieve approximately equal proportions of PPROM and sPTL cases are also identified. Controls are frequency matched on maternal age (e.g., within 5 years) and gestational age at blood collection (e.g., within 2 weeks).

The expression profile of the genes for preterm delivery genes can be determined by any of the methods known in the art and described above. In one embodiment, analysis of the expression profiles that make up the multimarker classifier is conducted using natural log-transformed data. For example, both supervised and unsupervised approaches may be used to identify inherent differences in gene expression patterns between sPTD cases and term controls. Unsupervised methods, such as cluster or principal component analysis (PCA), or any other methods in microarray analyses, may be used. PCA may be used to reduce the high dimension microarray data to 2 or 3 dimensions for easy visualization thus allowing similar comparisons across samples. In one embodiment, cluster analyses may simultaneously group samples and genes that share similar expression patterns. The color representation of heat mapping from cluster analysis can be used to reveal unique gene signatures to distinguish various sub-groups of participants in a global genomic fashion. A phylogenetic tree of genes that are differentially expressed may be constructed, e.g., by Cluster or TreeView software, or a hierarchical clustering algorithm that utilizes the Pearson's correlation coefficient, for example.

In one embodiment, supervised approaches may be used to identify subsets of genes that can robustly distinguish PTD cases from controls. As non-limiting examples, support vector machine (SVM), the significance analysis of microarrays (SAM), and the Shrunken Centroids methods, may be used to classify disease status. Briefly, in SAM analysis, a score statistic is calculated for each gene based on a ratio of change in gene expression (numerator) to standard deviation in the data for that gene plus an adjustment to minimize the coefficient of variation and enable comparison across all genes (denominator). In another embodiment, permutations to estimate the percentage of genes identified by chance, false discovery rate (FDR), for genes with scores greater than an adjustable threshold are also used. The FDR, q-value of a selected gene corresponds to the FDR for the gene list that includes the gene and all genes that are more significant. In another embodiment, a direct approach to gene selection to build classifiers using a subset of genes in a SVM model may be used. For example, the RankGene system can be used to choose K genes with the largest absolute value of scores in an SVM model. The system takes into account several criteria such as t-test statistic, information gain, and variance of expression to determine the discriminative strength of individual genes.

In another embodiment, other analytical approaches to gene selection may also be used, for example, those that reduce the possibility of colinearity among the selected K genes to increase classifier performance. As other non-limiting examples, greedy forward selection, genetic algorithms, and/or gradient-based leave-one-out gene selection (GLGS) algorithms may be used.

In one embodiment, a preferred criteria for classifier gene selection may be defined a priori. For example, in certain embodiments genes that satisfy the following three criteria in comparisons between sPTD cases and controls can comprise the set of genes used in a particular embodiment: (1) Student's t-test p-value <0.001; (2) fold change differences ≧2.0; and (3), false discovery rates (FDR) ≦10% as using (SAM). Standa advocated by the PREBIC Group may also be followed.

In another embodiment, the performance of the classifier may be evaluated. For example, cross validation approaches such as the 10-fold cross validation approach may be used. In this approach, derivation data is divided into 10 equal parts, each with 12 samples. 11 parts of the data are selected as a “test or training set” from which a classification model with K gene can be constructed to confirm its prediction performance on the remaining excluded part. The decision call for each excluded sample tested can be made based on the prediction function/score provided by each method. For instance, the Shrunken Centroids methods can provide a predictive probability of being in the PTD group. The procedure can be repeated 12 times then the overall error rate will be estimated. The overall error will likely depend on the number of K genes in the model. Hence, this number may be varied by changing the tuning parameter when using the Shrunken Centroids method. The optimal number of genes, K, or equivalently the optimal tuning parameter may be chosen such that the overall error rate reaches its minimum. Permutation testing may be used to assess the significance of the observed error rate. Briefly, 60 samples will be randomly relabeled as belonging to the PTD group and the remaining 60 in the term control group. The same 10-fold cross validation analysis as previously described may be conducted, and overall error rates recorded based on the optimal K genes from this permuted data. This procedure may be repeated as necessary, e.g., 10, 100, 1,000, 5,000 times (or any number in between) to obtain a null distribution of the overall error rate. Any other methods to measure the significance of overall error rates in the derivation set with correct classification may be used. For example, methods that can trade off bias for low variance, such as balance bootstrap re-sampling approaches, which have been shown to be a variance reducing technique, may also be used.

In another embodiment of the present invention, microarray findings are confirmed, e.g., using qRT-PCR methods. As a non-limiting example, a plurality of genes (e.g., 1, 2, 3, 4, 5, up to 50, or any number in between; preferably, 1-20 genes) may be selected for confirmation using methods such as qRT-PCR. qRT-PCR for the selected genes in the derivation set can be performed on all samples in both the derivation and the validation set. Correlation coefficients (e.g., Spearman's correlation coefficients) of expression values from microarray and qRT-PCR approaches can then be assessed.

In another embodiment of the present invention, the observed error rate for the samples in the validation data set can be calculated based on the classifier constructed from the independent samples from the derivation data set. A sPTD status label may be permuted on the derivation set to obtain a null classifier and validate its prediction performance on the validation data set. This procedure may be repeated as necessary, e.g., 10, 100, 1,000, 5,000 times (or any number in between) to obtain significance levels of the observed error rates. Alternatively, other methods of testing classification accuracy, such as PCA and multi-dimensional scaling (MDS) may be used. In one embodiment, a 2 (sPTD versus TERM) or 3-dimensional PCA of the validation samples based on the K genes in the classifier may be constructed from the derivation set.

In another embodiment, bioinformatics approaches may be used to retrieve and interpret complex biological interactions of the multimarker classifier. For example, Database for Annotation, Visualization and Integrated Discovery (DAVID) and Ingenuity Pathway Analysis (IPA) software (Ingenuity, Redwood City, Calif.) may be used to study systems biology and to explore mechanistic hypotheses. For example, an analysis based on DAVID can provide a comprehensive set of functional annotation tools and an enrichment analytic algorithm technique to identify enriched functional-related gene groups. A modified Fisher Exact p-value, an EASE score, can be used to measure the gene-enrichment in annotation terms by comparing the proportion of genes that fall under each category or term to the human genome background. An overall enrichment score for the group can be derived as the geometric mean (in log scale) of members' p-values (EASE score) in a corresponding annotation cluster. As another example, using analysis based on IPA, Ingenuity Pathways Knowledge Base (IPKB), a published and peer-reviewed database and computational algorithms can be used to identify local networks that are particularly enriched for the Network Eligible Genes, which can be defined as genes in our list of differentially expressed genes with at least one previously defined connection to another gene in the IPKB. A score that takes into account the number of Network Eligible Genes and the size of the networks, can be calculated using a Fisher Exact test as the negative log of the probability that the genes within that network are associated by chance. For example, a score of 3 (p-value corresponding to 0.001) as the cutoff for significance of the network can be used. The overall enrichment score in the analysis conducted using DAVID and the network score obtained in IPA can then be used to rank the biological significance of gene function clusters and networks, respectively, in PTD.

### Comparison

In one embodiment of the present invention, a set of expression profiles of preterm delivery marker genes in a biological sample from a subject are compared to a multimarker classifier. As one example, the expression profile is determined prior to the comparing step. As one example, the expression profile is of at least 1, 5, 10, 15, 20, 25, 30, 35, 40, 45, 50, 100, 150, 200, 250, 300, 350, 400, 450, 500, 550, 600, or any number in between 1 and 611, of the preterm delivery marker genes listed in Table 1. As another example, the expression profile is of at least 5, 10, 15, 20, 25, 30, 35, 40, 45, 50, 100, 150, 200, or any number in between 1 and 253, of the preterm delivery marker genes listed in Table 2. As another example, the expression profile is of at least 1, 5, 10, 15, 20, 25, 30, 35, 40, 45, 50, 55, 60, 65, or any number in between 1 and 69, of the preterm delivery marker genes listed in Table 3. As yet another example, the expression profile is of at least 1, 2, 3, 4, 5, 6, 7, 8, 9, 10, 11, 12, 13, 14, 15, 16, 17, 18, 19, 20, 21, 22, 23, 24, 25, 26, or 27 of the preterm delivery marker genes listed in Table 4.

In another example, the expression profile is of at least 1%, 5%, 10%, 20%, 30%, 40%, 50%, or between 1% and 50% of the preterm delivery marker genes listed in Table 1. As another example, the expression profile is of at least 1%, 10%, 20%, 30%, 40%, 50%, 60%, 70%, 80%, 90%, 100%, or any percent in between 1% and 100%, of the preterm delivery marker genes listed in Table 2. As another example, the expression profile is of at least 1%, 10%, 20%, 30%, 40%, 50%, 60%, 70%, 80%, 90%, 100%, or any percent in between 1% and 100%, of the preterm delivery marker genes listed in Table 3. As another example, the expression profile is of at least 1%, 10%, 20%, 30%, 40%, 50%, 60%, 70%, 80%, 90%, 100%, or any percent in between 1% and 100%, of the preterm delivery marker genes listed in Table 4. In another example, the expression profile is of 5 to 500 genes, 5 to 400 genes, 5 to 300 genes, 5 to 200 genes, 5 to 75 genes, 5 to 50 genes, 5 to 40 genes, 5 to 30 genes, 5 to 20 genes, 5 to 10 genes, or any other number in between 5 to 500 genes in a biological sample comprising peripheral blood cells.

Preferably, in the comparison of each gene in the expression profile to the same gene in the classifier, a gene identified as being upregulated or downregulated in a biological sample according to the invention is regulated in the same direction and to at least about 5%, and more preferably at least about 10%, and more preferably at least 20%, and more preferably at least 25%, and more preferably at least 30%, and more preferably at least 35%, and more preferably at least 40%, and more preferably at least 45%, and more preferably at least 50%, and preferably at least 55%, and more preferably at least 60%, and more preferably at least 65%, and more preferably at least 70%, and more preferably at least 75%, and more preferably at least 80%, and more preferably at least 85%, and more preferably at least 90%, and more preferably at least 95%, and more preferably of 100%, or any percentage change between 5% and higher in 1% increments (i.e., 5%, 6%, 7%, 8% . . . ), of the level of expression of the gene that is seen in the multimarker classifier. A gene identified as being upregulated or downregulated in an expression profile according to the invention can also be regulated in the same direction and to a higher level than the level of expression of the gene that is seen in the multimarker classifier.

The values obtained from the biological sample and multimarker classifier are statistically processed using any suitable method of statistical analysis to establish a suitable baseline level using methods standard in the art for establishing such values. Statistical significance according to the present invention should be at least p<0.05.

Those of skill in the art will appreciate that differences between the expression of genes may be small or large. Some small differences may be very reproducible and therefore nonetheless useful. For other purposes, large differences may be desirable for ease of detection of the activity. It will be therefore appreciated that the exact boundary between what is called a positive result and a negative result can shift, depending on the goal of the screening assay and the genes to be screened. For some assays it may be useful to set threshold levels of change. One of skill in the art can readily determine the criteria for screening given the information provided herein.

The level of expression of the gene or genes detected in the biological sample of the invention is compared to the baseline or control level of expression of that gene in the multimarker classifier. More specifically, according to the present invention, a “baseline level” is a control level of biomarker expression in the multimarker classifier against which a test level of biomarker expression (i.e., in the biological sample) can be compared. In one embodiment, control expression levels of genes of the multimarker classifier have been predetermined, such as for the genes listed in Tables 1-4. Such a form of stored information can include, for example, but is not limited to, a reference chart, listing or electronic file of gene expression levels and profiles for preterm delivery marker genes, or any other source of data regarding baseline biomarker expression that is useful in the methods disclosed herein. Therefore, it can be determined, based on the control or baseline level of biomarker expression or biological activity, whether the expression level of a gene or genes in a biological sample is/are more statistically significantly similar to the baseline multimarker classifier of preterm delivery marker genes. A profile of individual gene markers, including a matrix of two or more markers, can be generated by one or more of the methods described herein. According to the present invention, a profile of the genes in a biological sample refers to a reporting of the expression level of a given gene from Tables 1, 2, 3 or 4. The data can be reported as raw data, and/or statistically analyzed by any of a variety of methods, and/or combined with any other prognostic marker(s).

### Providing A Risk Assessment

In one embodiment of the present invention, a risk assessment for preterm delivery is provided. The risk assessment may be an output from the comparison of a set of expression profiles of preterm delivery marker genes in a biological sample to a multimarker classifier, as described above. The risk assessment may provide a dichotomous output (yes/no), a probability score, or a risk classification, as non-limiting examples. For example, the risk assessment may provide a dichotomous yes/no output as to whether the subject from whom the biological sample was obtained will or will not deliver preterm. The risk assessment may provide a yes/no output as to whether or not the subject is at risk of a particular type of preterm delivery, e.g., VPTD, MPTD, sPTL, or PPROM, or any combination thereof. For example, the risk assessment may provide a probability score, e.g., a number on a relative scale indicating likelihood of delivering preterm, or other type of indicator (i.e., no risk, low risk, medium risk, high risk, very high risk). As another example, the probability score may provide a score for a particular type of preterm delivery, e.g., VPTD, MPTD, sPTL, or PPROM, or any combination thereof. As another example, the risk assessment may also provide a preterm delivery risk classification based on the expression levels of various preterm delivery marker genes.

### Obtaining or Storing a Biological Sample

In one embodiment, a biological sample is obtained prior to determining the set of expression profiles. A biological sample may be, for example, a blood sample, preferably, a whole blood sample, or any sample containing peripheral blood cells. For example, a 20-ml non-fasting blood sample may be collected. Blood may be drawn into a 10 ml plain red-top vacutainer and a 10 ml lavender-top vacutainer containing K₃-EDTA (1 mg/ml). Blood in the plain vacutainer may be allowed to clot at ambient temperature and is then centrifuged to recover serum. Serum can be aliquoted and stored at −80° C. until analysis. In one embodiment, a mononuclear blood cell fraction may be isolated from the biological sample. In another embodiment, lymphocytes may be isolated from the biological sample. In another embodiment, a cell fraction enriched for mononuclear blood cells may be obtained from the biological sample. In another embodiment, a cell fraction enriched for lymphocytes may be obtained from the biological sample. For example, the lavender-top vacutainer may be centrifuged at 85 g for 20 minutes at 4° C. to separate the red cells, white cells, and plasma. Fractions may be aliquoted and stored at −80° C. until analysis. Urine samples may also be collected at this time. Samples may be immediately aliquoted and stored at −80° C. until analysis.

The biological samples may be collected antepartum from mothers in early pregnancy. For example, the samples may be collected from mothers prior to 20 weeks gestation, prior to 16 weeks gestation, between 13-16 weeks gestation, within the first trimester of pregnancy, second trimester, or third trimester of pregnancy. Preferably, the sample is collected within the first trimester of pregnancy. Alternatively, the samples may be collected from non-pregnant women.

Once a biological sample is obtained, it may then be used to determine a set of expression profiles of preterm delivery marker genes using any of the steps described herein.

### Therapies

In one embodiment of the present invention, a subject indicated to have a high risk of preterm delivery may be prescribed or provided with a prophylactic therapy for reducing the risk of preterm delivery. For example, a subject may be treated with progesterone therapy to reduce the risk of preterm delivery, an anti-inflammatory therapy to alleviate inflammation associated with the risk of preterm delivery, or an anti-diabetic therapy to control the subject's glucose or metabolic levels associated with the risk of preterm delivery, or a combination thereof. Further, a subject may be treated with a therapy to reduce oxidative stress, intravascular hemolysis, endothelial dysfunction, or any other metabolic alteration associated with a high risk of preterm delivery.

### EXAMPLES

The following specific examples are illustrative, but do not limit the remainder of the disclosure of the invention in any way whatsoever.

### Example 1

### Sample Collection

Information was collected from subjects participating in an ongoing prospective cohort study conducted at the Center for Perinatal Studies (CPS) at Swedish Medical Center in Seattle, Wash. The Omega Study (5R01HD032562-10) was designed primarily to examine the metabolic and dietary predictors of preeclampsia, gestational diabetes, and other pregnancy outcomes. Briefly, Omega Study participants were recruited from women attending prenatal care at clinics affiliated with Swedish Medical Center. Women who initiated prenatal care prior to 20 weeks gestation were eligible to participate. Women were ineligible if they were younger than 18 years of age, did not speak and read English, did not plan to carry the pregnancy to term, did not plan to deliver at the research hospital, and/or were past 20 weeks gestation. Nine years after beginning recruitment, approximately 81% of approached women consented to participate and 96% were followed through pregnancy completion. More than 4,050 participants have been enrolled. The study population used consisted of 1,600 women enrolled in the Omega Study from whom whole blood samples suitable for conducting antepartum whole genome gene expression profiling were collected and stored.

TABLE 5
Omega Study Data Collection

| Timing of Collection | Method of Collection | Data Collected |
|---|---|---|
| Enrollment (13 weeks | (1) Interviewer-administered | Socio-demographics |
| gestation, on average) | questionnaire | Lifestyle and behaviors, Medical history |
| | | Reproductive history, perceived and |
| | | actual stress |
| | (2) Food frequency questionnaire | Periconceptional dietary intake |
| | (3) Non-fasting blood draw | Plasma and serum |
| | (4) Paxgene RNA stabilization tubes | Whole blood (since 2005) |
| | (5) Urine sample collection | Urine |
| Post-delivery | (6) Medical record abstraction | Pregnancy course and outcome |
| | | Placenta (since 2005), Cord blood (since |
| | | 2005) |
| | | Prenatal care, ante/postpartum |
| | | complications |
| | | Preeclampsia & preterm delivery |
| | | occurrence |

Omega Study data collection is summarized in Table 5. At or near enrollment (13 weeks gestation), in-person interviews were conducted. These questionnaires of 45-60 minutes in length were administered in English by trained interviewers. Collected data included sociodemographic characteristics, occupation, reproductive and medical histories, alcohol and tobacco consumption, environmental tobacco smoke exposure, medications, height, weight and weight gain, physical activity before and during pregnancy, and familial histories of medical conditions. At or near the time of interview (13 weeks gestation), trained phlebotomists collected a non-fasting blood sample from each participant. Blood was drawn into plain and vacutainer tubes containing K₃-EDTA (1 mg/mL). Blood in the plain vacutainer was allowed to clot at ambient temperature and was then centrifuged to recover serum. Serum was aliquoted and stored at −80° C. until analysis. The EDTA tube was centrifuged at 850 g for 20 minutes at 4° C. to separate the red cells, white cells, and plasma. Fractions were aliquoted and stored at −80° C. until analysis. Beginning in 2005, we modified our sample collection and storage procedures to collect antepartum whole blood in PAXgene™ Blood RNA System tubes. The system enabled the consolidation of key steps of whole blood collection, nucleic acid stabilization, and RNA purification. Urine samples were also collected at this time. Samples are immediately aliquoted and stored at −80° C. until analysis. After delivery, trained personnel abstract data from Omega participants' maternal and infant medical records. These data will be used to ascertain pregnancy outcomes (described in detail below).

The distribution of the first 2,000 participants is presented in Table 6. Subjects were included in the study regardless of race/ethnicity. Omega study participants were similar to those enrolled in other selected pregnancy cohorts conducted in different regions of the U.S. The Omega population, like the New Haven cohort, was mostly White and well educated. The population was older on average than women giving birth in WA State (average 28 years) and Pittsburgh. Omega participants were less likely than women in North Carolina and Pittsburgh cohorts to smoke during pregnancy. Overall, women enrolled in our cohort were similar to those enrolled in other cohorts.

TABLE 6
Study Population Characteristics from Five
Prospective Cohort Studies of Pregnancy

| | Omega¹ | PEPPS² | PIN³ | Yale⁴ | Camden⁵ |
|---|---|---|---|---|---|
| Maternal age (years) | | | | | |
| Mean | 32 | 25 | — | 31* | — |
| Median | 32 | — | 25 | — | — |
| % 18 or younger | <1 | — | — | — | 56 |
| % 35 or younger | 29 | — | 10 | 20 | — |
| White race (%) | 85 | 63 | 54 | 90 | 13 |
| Post high-school education | 95 | 43 | 52† | 82 | — |
| Nulliparous (%) | 67 | 58 | 45† | 44 | 58 |
| Primigravid (%) | 41 | 42 | 29 | 27 | — |
| Prior abortion | | | | | |
| Prior spontaneous abortion | 23 | — | 21 | — | — |
| ($) | | | | | |
| Prior induced abortion (%) | 24 | — | 26 | — | — |
| Maternal adiposity | | | | | |
| Mean pre-pregnancy BMI | 24 | 25 | — | 24* | — |
| (kg/m²) | | | | | |
| Pre-pregnancy weight > | 31 | — | — | 24 | — |
| 15 lb (%) | | | | | |
| Pre pregnancy BMI > | 19 | — | — | — | 22 |
| 26.0 kg/m² (%) | | | | | |
| Pre pregnancy BMI > | 12 | — | 25† | — | — |
| 29.0 kg/m² (%) | | | | | |
| Smoked during pregnancy | 22 | 32 | 29 | 14 | 21 |
| (%) | | | | | |

¹From the first 2,000 participants of the Omega Study.
²From 1,984 normotensive participants of the Pregnancy Exposures and Preeclampsia Prevention Study (50).
³From 2,806 participants of the Pregnancy, Infection, and Nutrition (PIN) Study (89) or, if crossed, from 2,319 PIN participants with complete covariate information (65).
⁴From 2,714 participants of the Yale Health in Pregnancy Study (27) or, if starred, from 2,422 normotensive Yale Study participants (64).
⁵From 2,073 participants of the Camden study (66).

### Example 2

### Global Gene Expression Profiling in Whole Blood: Obese & Lean Women in Early Pregnancy

Adiposity is consistently identified as an important risk factor of adverse pregnancy outcomes. Adipose tissue, once thought to be an inert depot of energy, is now recognized to exert considerable influence on glucose handling and other metabolic processes.

In this preliminary study, we investigated whether maternal pre-pregnancy obesity was associated with biologically relevant alterations of mRNA expression profiles of genes involved in endocrine, inflammatory and other processes. Maternal whole blood mRNA samples collected during early pregnancy (16 weeks on average) from 10 obese (BMI ≧30) and 10 lean women (BMI <20) were compared using Affymetrix Human Focus GeneChip arrays. The complete set of arrays was normalized and background corrected using GC-RMA. Array sensitivity was determined using a set of spiked controls and a lower-bound signal intensity threshold was established. Probe sets that did not exceed the threshold were removed from further analysis. Significant changes in expression between experimental and control samples were determined using the Welsh t-test.

This analysis identified 104 genes that were differentially expressed among lean and obese women (p-value<0.05). Among these genes were members of the immune response (n=4), coagulation (n=2), and oxidative stress (n=8) pathways, which are all affected by obesity. These results indicated that a blood-based gene expression study could be done in early pregnancy and that expression patterns may be used to identify and evaluate relevant etiologic and mechanistic hypotheses in perinatal epidemiology studies.

### Example3

### Global Gene Expression Profiling in Placentas from Preeclampsia and Normotensive Patients

Preeclampsia is a pregnancy-related vascular disorder characterized by hypertension and proteinuria. The central pathology characterizing preeclampsia, failure of implantation due to impaired trophoblast invasion and endothelial dysfunction, involves the placenta. Various pathways including oxidative stress, inflammation, growth regulation, angiogenesis, tumor suppression, apoptosis, immune tolerance, coagulation and lipid metabolism have been shown to be relevant in the pathogenesis of preeclampsia.

We compared the global gene expression (˜22,000 genes) profiles of 36 placentas using oligonucleotide microarray technologies (18 preeclampsia cases and 18 normotensive term controls). RNA isolation and microarray analyses were completed. Statistical analyses were performed on natural log-transformed data.

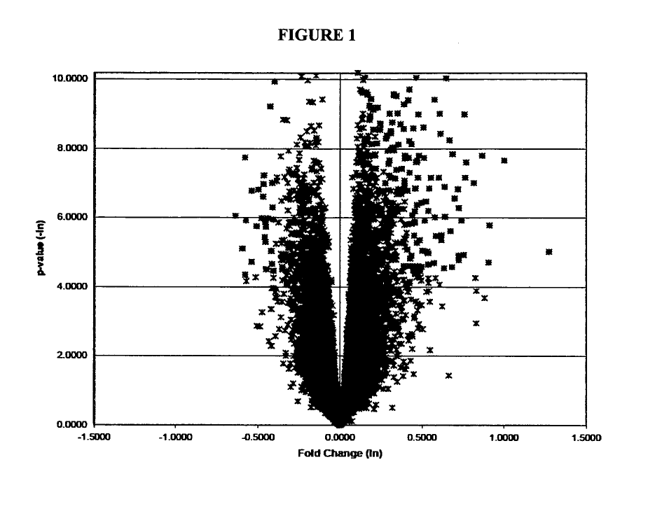

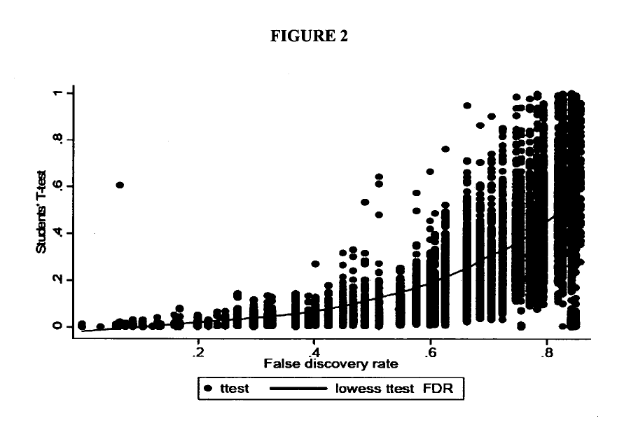

Approximately 96.6% (21,250 of 22,000 genes on our oligonucleotide microarray platform) were expressed in study tissue. We used students' T-test, fold change and Significance Analysis of Microarrays (SAM) to identify genes that were differentially expressed in preeclampsia versus control tissues. Results are shown in FIGS. 1 and 2.

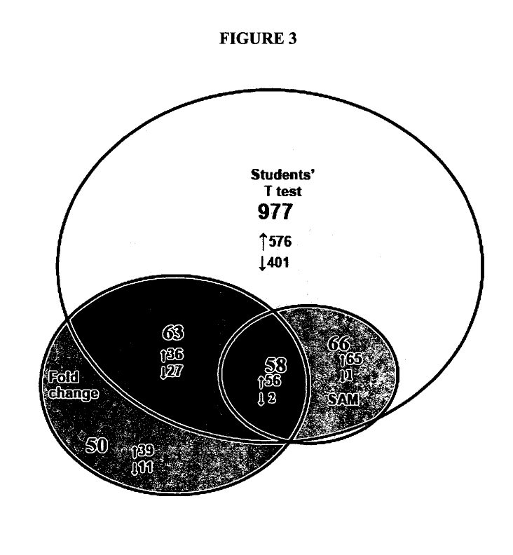

As summarized in FIG. 3, several genes were identified as being differentially expressed (772 up-regulated and 442 down-regulated) by at least one of the methods used to evaluate the differences in gene expression between placenta of preeclampsia cases and normotensive controls. The Students' t-test analytical approach proved to be the most permissive approach, identifying 1,164 genes, 733 up-regulated and 431 down-regulated genes. The SAM FDR approach appeared to provide the most stringently defined group of differentially expressed genes, identifying 124 genes, 121 up-regulated and 3 down-regulated (FIG. 3). The simple fold-change analytical approach, identified 171 differentially expressed genes (131 up-regulated and 40 down-regulated). A total of 58 genes, 56 up regulated and 2 down-regulated, met all three criteria. These 58 differentially expressed genes comprised our set of genes identified as requiring further detailed analysis for preeclampsia studies.

Genes that satisfied the following three criteria in comparisons between cases and controls constituted the final set of genes that were differentially expressed in the placental tissue in preeclampsia. These criteria were Student's t-test p-value <0.05, fold change differences ≧1.5, and, false discovery rates (FDR) ≦10% calculated using Significance Analysis of Microarrays (SAM).

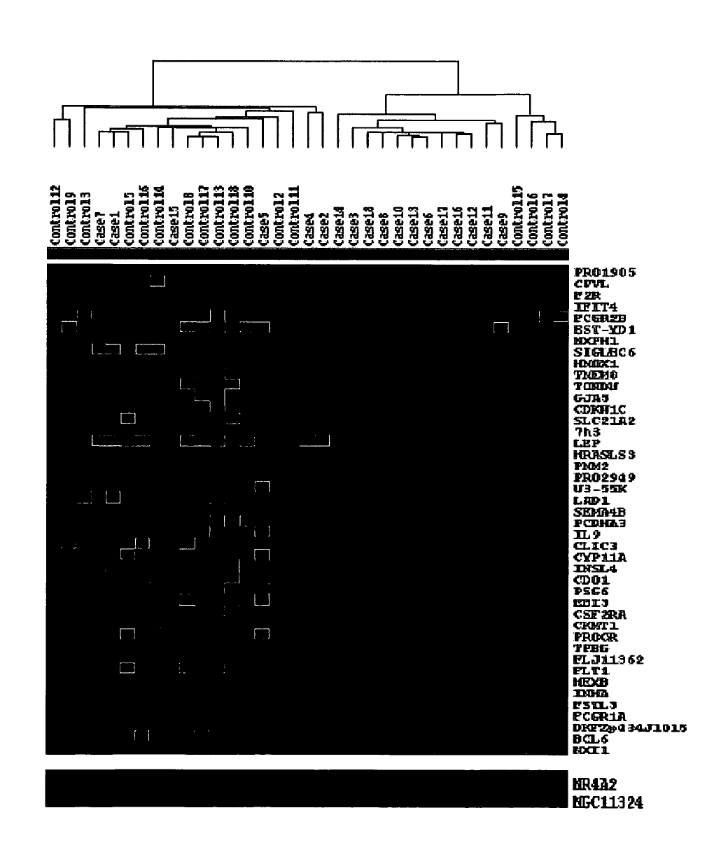

We used Cluster and TreeView software to construct a phylogenetic tree of differentially expressed genes. The programs used hierarchical clustering approaches based on the Pearson's correlation coefficient estimates. Results are summarized in FIG. 4. Cluster analysis of samples and 58 differentially expressed genes, depicted by the heat map in FIG. 4, resulted in a similar 78% sensitivity (14/18 of cases grouped together) and specificity (14/18 controls grouped together).

Genes with significant a priori evidence for involvement in preeclampsia pathology (such as, LEP, FLT1, INHA and F2R), as well as genes for which limited previous evidence exists, but were potential candidates for their roles in pathways previously associated with preeclampsia (such as CYP11A, FCGR2B, HMOX1, PSG6, CDKN1C and TPBG) were identified in our pilot study.

To further investigate the biological processes involved in preeclampsia pathogenesis, we performed Path Analyses using two powerful independent bioinformatics programs. We used the Database for Annotation, Visualization and Integrated Discovery (DAVID) software and the Ingenuity Pathway Analysis (IPA) software (Ingenuity, Redwood City, Calif.). Results are presented in Tables 7 and 8.

TABLE 7
DAVID Mapping of Genes Differentially Expressed in Preeclampsia Placenta

| Gene List | Enrichment Score | Cluster |
|---|---|---|
| PSG6, INHA, FLT1, INSL4 | 2.57 | Reproductive organismal physiological process, |
| | | reproductive physiological process, reproduction |
| FCGR1A, FCGR2B, IL9, INHA, | 2.24 | Immune response, defense response |
| EBI3, NR4A2, PROCR, IFIT4, | | |
| BCL6 | | |
| FCGR1A, FCGR2B, PSG6, | 2.02 | Domain: Ig-like C2-type 2, domain: Ig-like C2- |
| SIGLEC6, FLT1 | | type 1, Immunoglobulin, Immunoglobulin C2 |
| | | type, Immunoglobulin subtype, IG, |
| | | Immunoglobulin-like |
| PSG6, INSL4 | 1.88 | Pregnancy, physiologic interaction between |
| | | organisms, interaction between organisms |
| IL9, INHA, EBI3 | 1.66 | Regulation of cytokine biosynthesis, cytokine |
| | | biosynthesis, regulation of cytokine production, |
| | | cytokine metabolism, cytokine production, |
| | | regulation of immune response, regulation of |
| | | protein metabol. Cytokine activity |
| CDKN1C, INHA, MXI1, | 1.38 | Negative regulation of cellular physiological |
| HRASLS3, BCL6 | | process, negative regulation of physiological |
| | | process |
| CDKNIC, INHA, HRASLC3, | 1.22 | Cycle, regulation of cell cycle, cell cycle |
| F2R | | |

*GenBank accession numbers were mapped using functional annotation clustering in the DAVID 2007 pathway analysis tool. For each group, the processes or functions are tabulated with the gene list and enrichment score. Enrichment score is calculated as the geometric mean (in log scale) of members' p-values in a corresponding annotation cluster. Clusters shown here are those with enrichment scores >1.0.

TABLE 8
Gene Clusters Identified Using Ingenuity Path Analysis in Preeclampsia Placenta

| Genes in Network (Genes from our set are shown in | | Focus | |
|---|---|---|---|
| bold) | Score | Genes | Functions |
| AKT2, ASS1, AZGP1, beta-estradiol, CASP14, CDO1, | 30 | 14 | Cellular development, |
| CTSH, CYP24A1, FSTL3, GAL, GCLC, HEXB, | | | Hematological System |
| HRASLS3, IFIT3, IFNG, IGFBP4, IGSF1, INHA, MBP, | | | Development and Function, |
| MOG, MXI1, MYOD1, NPY1R, NXPH1, PCSK1, | | | Immune and Lymphatic |
| PMM2, prostaglandin E2, PYGM, SLCO2A1, TCF12, | | | System Development and |
| TEAD2, TGFB1, TNFRSF11B, VGLL1, ZFP36 | | | Function |
| ADRB3, Akt, ASS1, BCL6, BSG, CDKNIC, CPTIA, | 28 | 13 | Cell Death, Cellular Growth |
| CSF2RA, DLL4, EBI3, F2R, FCGR1A, FCGR2B, FLT1, | | | and Proliferation, |
| GCLC, HMOX1, IL9, IL5RA, IL9R, LEP, Mapk, NR4A2, | | | Inflammatory Disease |
| P38 MAPK, PDE3B, PMCH, PRKAA1, RETN, | | | |
| RUNXIT1, SHC2 (includes EG: 25759), SLC25A5, | | | |
| STAT5a/b, UCN, VEGF, VEGF Receptor, VEGFB | | | |
| (includes EG: 7423) | | | |
| 4-androstene-3, 17-dione, ABCB11, ABCC3, ASS1, carbon | 8 | 5 | Cellular Growth and |
| monoxide, CYP11A1, CYP2E1, CYP7A1, FCGR2B, | | | Proliferation, Small |
| GCLC, GHRL, GRN, Gsk3, HMGCR, HSD11B1, IGFBP4, | | | Molecule Biochemistry, |
| IL27, IL10RA, IL1B, LAD1, Nos, NR4A2, NR4A3, | | | Connective Tissue |
| ORM2, PGF, PROCR, PRTN3, RPSA, SCARB1, SEPP1, | | | Development and Function |
| TFAP2C, TG, THBD, TNF, TNFRSF11B | | | |

*GenBank accession numbers were mapped using IPA software and using IPKB, genes are assigned to networks and network enrichment is assessed using a score (negative log of p-values of Fisher tests). Focus genes (in bold) are genes identified in our list of differentially expressed genes.

Assessment using DAVID showed that genes in our list belonged to cluster of genes involved in reproductive physiology, immune responses, and cytokines. To a lesser extent, genes involved in negative cell function regulation and cell cycle were also represented in our set of genes. Assessment using IPA showed that networks involving cellular development, particularly of the hematological, lymphatic, connective tissue and immune systems as well as inflammatory disease were particularly enriched by genes in our set of differentially expressed genes.

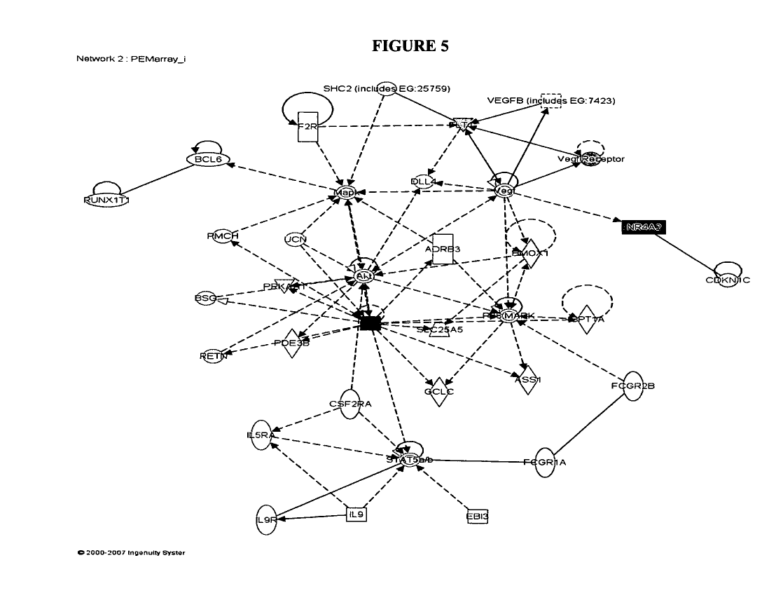

One network that was strongly identified (score 28) using our differentially expressed gene list and IPA software is depicted in FIG. 5. Some genes, besides the already identified ones, seemed to play a central role in these networks. These genes included transforming growth factor-β1 (TGFB1), tumor necrosis factor receptor-1 (TNFRSF11B), interferon gamma (IFNG), MYOD1, prostaglandin E₂ and β-estradiol from Network 1 and genes AKT, MAPK, P38MAPK, STAT5a/b and vascular endothelial growth factor (VEGF) from Network 2.

In this global placental gene expression study, 58 genes were differentially expressed between preeclampsia cases and controls. These genes participate in a diverse set of cellular functions reflecting involvement of several pathways in preeclampsia pathogenesis. These functions included cellular growth, inflammation, oxidative stress, tissue development (especially of the hematological system), signaling, and hormone metabolism. Genes with significant a priori evidence for involvement in preeclampsia pathology (such as LEP, FLT1, INHA, and F2R), as well as genes for which limited previous evidence exists, but were potential candidates for their roles in pathways previously associated with preeclampsia (such as CYP11A, FCGR2B, HMOX1, PSG6, CDKN1C and TPBG) were identified. Further, path analysis results provided evidence for involvement of other potential candidate genes in preeclampsia pathogenesis including TGFB1, TNFRSF11B, AKT and P38MAPK, although expression of these genes were not different between cases and controls in the current study.

### Example 4

### Identification of Gene Expression Involved in Preterm Delivery

This case-control study was to demonstrate the feasibility of comparative maternal whole blood transcriptome studies using maternal samples collected in early pregnancy.

We sought to identify differences in patterns of gene expression in peripheral blood cells (PBLs) among 14 women distained to deliver preterm (spontaneous preterm delivery 20 to <35 weeks gestation; sPTD) compared with 16 women who subsequently delivered at term (≧37 weeks gestation). We also constructed a multi-marker classifier (antepartum [16 weeks gestational age] whole blood gene expression profile) that will serve to identify women at high risk of sPTD using Affymetrix Human Genome U133 Plus2.0 Arrays.

We identified a total of 611 genes (Table 1) that were statistically significantly differentially expressed in maternal early pregnancy PBL by a magnitude of 1.5-fold or greater between women who would go on to deliver preterm term versus those who delivered at term. A more stringent gene list (list of genes with a 2-fold change (FC) cut-off for expression-level change between the preterm and control groups yielded a list of 87 genes. We assessed functions and functional relationships of differentially expressed genes using DAVID software. Genes participating in cell signaling, immune response, oxidative stress response and regulation of cell death, were differentially expressed in PBL of sPTD cases. Genes include those with strong a priori evidence for involvement in sPTD pathogenesis and novel genes.

We generated various gene lists using Principle Components Analysis (PCA) and/or two-Dimensional Hierarchical Clustering analysis (2D clustering) methods in order to distinguish sPTD and term samples. Student t-test was performed and a P-value <0.01 level was established to filer genes. Using these stringent criteria we identified 69 genes (Table 3) with over 1.5-fold average mean difference between sPTD and term study groups. Notably, we identified 17 genes with over 2-fold average mean difference between the two groups (these 17 genes are included in the 69-gene list).

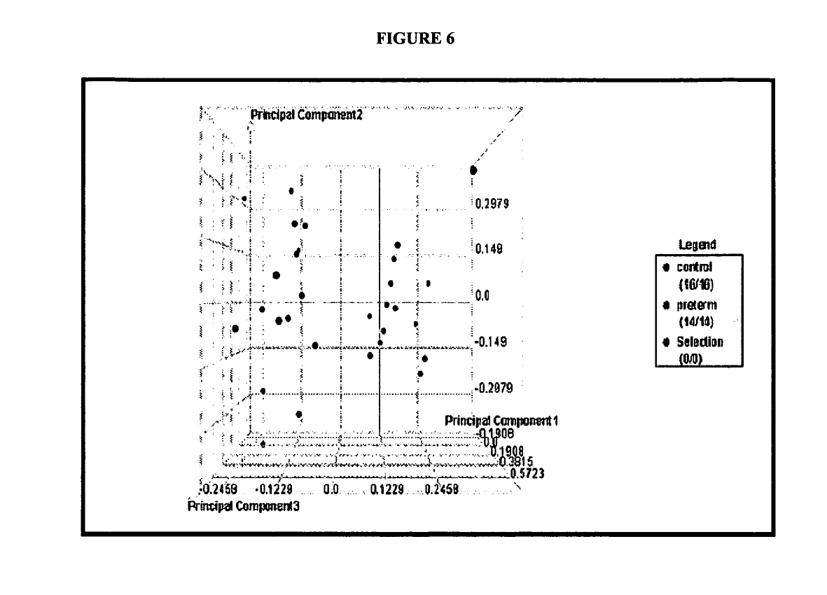

FIG. 6 shows PCA results from the 69 genes (P<0.01, 1.5-FC) and 30 arrays. The arrays were separated into their corresponding study group.

### Example 5

### Construction of Multi-Marker Classifier for Preterm Delivery

Whole blood samples for stabilization of mRNA for PBLs are collected. Analyses of gene expression is performed for 60 PTD cases and 60 term controls to construct a multi-marker classifier for sPTD. To minimize potential confounding by maternal race/ethnicity and to optimize the statistical power of our research, analyses are restricted to non-Hispanic White and African-American women. Women with multi-fetal gestation and women delivering infants with malformations are also excluded. To control for potential confounding by maternal age and gestational age in collection of whole blood, selected sPTD cases are frequency matched to term controls on age (within 5 years) and timing of blood collection (within 2 weeks). Study findings are confirmed in an independent data set of 60 sPTD cases and 60 term controls. Therefore, identical exclusion/selection and frequency matching criteria are used to select participants for independent validation analyses.

All laboratory procedures are completed without knowledge of case or control status. After isolation of mRNA, gene expression profiling is performed on the 120 participants in the derivation set and the 120 participants in the validation set. Data analysis of the derivation set focuses on identification of clusters of genes with differential expression between sPTD cases and control. There is also be a focus on constructing classifiers—identifying groups of selected genes that optimize discrimination of sPTL versus PPROM cases. qRT-PCR procedures are used to verify microarray results. Up to 20 genes are selected that are most differentially expressed between sPTD cases and controls (and which are most differentially expressed between sPTL cases and PPROM cases) to confirm the classifier.

Expression profiles of individuals in the validation set are evaluated for classification of sPTD cases versus controls (sPTL cases versus PPROM cases) based on results from the derivation data set. Specific gene sets related to biologic pathways will be evaluated for which expression is differentially regulated for sPTD cases and term controls in both the derivation and validation data set. Initial analyses are completed separately, and repeated on the combined data set.

Whole Blood Collection and isolation of RNA. PAXgene™ Blood RNA tubes and Blood RNA Kit (PreAnalytiX, Qiagen, Inc) are used for collection of whole blood (5 ml) and stabilization, purification, and isolation of RNA. Total mRNA is isolated from whole blood samples using the PAXgene Blood RNA Kit (Qiagen Inc., Valencia, Calif.) following standard procedures. Total RNA concentrations are calculated by determining absorbance at 260 nm (Spectramax Plus 384 spectrophotometer, Molecular Devices, Sunnyvale, Calif.) in 10 mM Tris-HCl. Protein contamination is monitored using the A260/A280 ratio. To assure high quality, all samples have an A260/A280 ratio of >1.8. The GLOBINclear kit (Ambion, Austin, Tex.) is used to decrease the masking effect abundant globin mRNA has on less abundant mRNA. Purified RNA samples are used to perform microarray experiments or immediately stored frozen in a buffer at −80° C. for qRT-PCR experiments designed to verify microarray results.

Samples are assessed for quality control and fluorescently labeled. Quality control of total RNA is analyzed using an Agilent 2100 Bioanalyzer capillary electrophoresis system, and spectrophotometric scan of each sample in the UV range from 220-300 nm. Those RNA samples that pass QC are amplified using Ambion's MessageAmp I kit and the subsequent RNA labeled with a fluorescent dye tag. RNA samples, including reference RNAs, are QC'ed, amplified, and labeled using standardized protocols.

Commercially printed microarrays having the 4×44 k slide format (probes are 60-mers and the array format is two-channel) from Agilent Technologies (Santa Clara, Calif.) are used. Array information is obtained from RefSeq, Goldenpath Ensembl Unigene Human Genome (Build 33) and GenBank. Array processing protocols (i.e., hybridization and washes) are fully automated with the use of two Robbins Scientific Hybridization Incubator equipped with Agilent Technologies rotisserie assemblies. Protocols and reagents used are outlined at the web site www.chem.agilent.com/Scripts/PDS.asp?1Page=34519. Post-hybridized arrays are imaged using an Agilent Technologies DNA Microarray Scanner.

Array images are quantified, tested for signal quality and normalized using Agilent Feature Extraction Software v9.5.3 (Agilent Technologies). Statistical data analysis and data visualization are performed using GeneSpring 7.0 microarray analysis software (Agilent Technologies and open-source tools such as those provided by the BioConductor Bioinformatics Resource (www.bioconductor.org/).

Verification of expression data obtained from genomic microarrays is performed using qRT-PCR-based analyses for up to 20 genes identified as classifiers of sPTD. First strand cDNA is synthesized by using the High Capacity cDNA Archive Kit (Applied Biosystems, Foster City, Calif.). The reverse transcription reaction for each sample is performed either the day of or the day before the PCR reaction. This is so that cDNA will not be degraded by storage. Testing in our lab has shown that overnight storage of cDNA at 4° C. has negligible effects on PCR results. qRT-PCR is performed in duplicate on 25 μL mixtures, containing 25-150 ng of template cDNA, 12.5 μL of 2× Taqman Universal Master Mix (Applied Biosystems), and 1.25 uL of Taqman Gene Expression Assay for the gene of interest or control gene (Applied Biosystems). Assays that are reported by Applied

Biosystems (or the appropriate primer-probe set) to pick up genomic DNA are additionally tested for genomic DNA contamination by running a reverse transcriptase minus (RT−) control for every sample. Reactions are run in 96-well plates with optical covers (Applied Biosystems) on an ABI PRISM 7000 Real Time PCR machine (Applied Biosystems) using the default cycling conditions. Four point control cDNA is used for primer efficiency comparison of all Assays on Demand based on the slope of each standard curve calculated by the ABI PRISM 7000 SDS Software, Version 1.1.

Statistical and Bioinformatics Analysis. Analysis is conducted using natural log-transformed data. Both supervised and unsupervised approaches are used to identify inherent differences in gene expression patterns between sPTD cases and term controls. Unsupervised methods, such as cluster or principal component analysis (PCA), are commonly used in microarray analyses. PCA is used to reduce the high dimension microarray data to 2 or 3 dimensions for easy visualization thus allowing similar comparisons across samples. Cluster analyses simultaneously groups samples and genes that share similar expression patterns. The color representation of heat mapping from cluster analysis reveals unique gene signatures to distinguish various sub-groups of participants in a global genomic fashion. Cluster and TreeView software is used to construct a phylogenetic tree of genes (that are differentially expressed). The programs use a hierarchical clustering algorithm that utilizes the Pearson's correlation coefficient.

Although unsupervised methods provide the means to visualize global gene expression patterns, it is more appropriate to use supervised approaches to identify subset of genes that can robustly distinguish PTD cases from controls. The support vector machine (SVM), the significance analysis of microarrays (SAM), and the Shrunken Centroids methods are three candidate methods that are widely used to classify disease status. Permutations are also used to estimate the percentage of genes identified by chance, false discovery rate (FDR), for genes with scores greater than an adjustable threshold. The FDR, q-value of a selected gene corresponds to the FDR for the gene list that includes the gene and all genes that were more significant. Some investigators use a direct approach to gene selection to build classifiers using a subset of genes in a SVM model. This is done by choosing K genes with the largest absolute value of scores in an SVM model built using the RankGene system. The system takes into account several criteria such as t-test statistic, information gain, and variance of expression to determine the discriminative strength of individual genes. Although the aforementioned approaches are computationally straight-forward, investigators have noted that the possibility of collinearity among the selected K genes may reduce classifier performance; other analytical approaches to gene selection are also used. Data is analyzed using other methods such as greedy forward selection, genetic algorithms, and gradient-based leave-one-out gene selection (GLGS) algorithms. We recognize that use of multiple approaches will yield different results, so we have defined a priori our preferred criteria for classifier gene selection. Genes that satisfy the following three criteria in comparisons between sPTD cases and controls constitute the final set of genes: (1) Student's t-test p-value <0.001; (2) fold change differences ≧2.0; and (3), false discovery rates (FDR) ≦10% as using (SAM). We also follow the standards advocated by the PREBIC Group.

The “10-fold cross validation” approach is used on the derivation data set to evaluate the performance of classifiers identified. The derivation data is divided into 10 equal parts, each with 12 samples. 11 parts of the data are selected as a “test or training set” from which a classification model with K gene will be constructed to confirm its prediction performance on the remaining excluded part. The decision call for each excluded sample tested is made based on the prediction function/score provided by each method. For instance the Shrunken Centroids methods provide a predictive probability of being in the PTD (or sPTD or PPROM group). The procedure is repeated 12 times then the overall error rate will be estimated. The overall error depends on the number of K genes in the model. Hence, the number is varied by changing the tuning parameter when using the Shrunken Centroids method. The optimal number of genes, K, or equivalently the optimal tuning parameter is chosen such that the overall error rater reaches its minimum. Permutation testing is used to assess the significance of the observed error rate. Briefly, 60 samples are randomly relabeled as belonging to the PTD group and the remaining 60 in the term control group. Then the same 10-fold cross validation analysis as previously described is conducted, and overall error rates recorded based on the optimal K genes from this permuted data. This procedure is repeated 1,000 times to obtain a null distribution of the overall error rate, allowing us to measure the significance of overall error rates in the derivation set with correct classification. Exploratory analyses are conducted for estimating error rates. Methods that trade off bias for low variance, such as balance bootstrap re-sampling approaches, which have been shown to be a variance reducing technique, are used.

Microarray findings are confirmed using qRT-PCR methods. qRT-PCR for up to 20 genes, is performed on all 240 samples in both the derivation and the validation set. Correlation coefficients (e.g., Spearman's correlation coefficients) of expression values from microarray and qRT-PCR approaches are assessed.

The observed error rate for the 120 samples in the validation data set is calculated based on the classifier constructed from the 120 independent samples from the derivation data set. sPTD status label on the derivation set is permuted to obtain a null classifier and validate its prediction performance on the validation data set. This procedure is repeated 1,000 times, and significance levels of the observed error rates obtained. As an alternative means of testing classification accuracy, exploratory methods such as PCA and multi-dimensional scaling (MDS) are also used. A 2 (sPTD versus TERM) or 3-dimensional PCA (sPTL, PPROM, TERM) of the 120 validation samples based on the K genes in the classifier constructed from the derivation set is constructed.

Bioinformatics approaches are used to retrieve and interpret complex biological interactions. Two independent tools are used: (1) DAVID and (2) Ingenuity Pathway Analysis (IPA) software (Ingenuity, Redwood City, Calif.) to study systems biology and to explore mechanistic hypotheses. In analysis based on DAVID, a comprehensive set of functional annotation tools and an enrichment analytic algorithm technique are used to identify enriched functional-related gene groups. A modified Fisher Exact p-value, an EASE score, are used to measure the gene-enrichment in annotation terms by comparing the proportion of genes that fall under each category or term to the human genome background. An overall enrichment score for the group is derived as the geometric mean (in log scale) of member' p-values (EASE score) in a corresponding annotation cluster. In IPA, Ingenuity Pathways Knowledge Base (IPKB), a published and peer-reviewed database and computational algorithms is used to identify local networks that are particularly enriched for the Network Eligible Genes, defined as genes in our list of differentially expressed genes with at least one previously defined connection to another gene in the IPKB. A score, that takes into account the number of Network Eligible Genes and the size of the networks, is calculated using a Fisher Exact test as the negative log of the probability that the genes within that network are associated by chance. A score of 3 (p-value corresponding to 0.001) as the cutoff for significance of the network is used. The overall enrichment score in the analysis conducted using DAVID and the network score obtained in IPA is used to rank the biological significance of gene function clusters and networks, respectively, in PTD.

It is understood that the examples and embodiments described herein are for illustrative purposes only and that various modifications or changes in light thereof will be suggested to persons skilled in the art and are to be included within the spirit and purview of this application and scope of the appended claims.

1. A method for determining a risk of preterm delivery in a subject, comprising:
(i) comparing (a) a set of expression profiles of preterm delivery marker genes in a biological sample comprising peripheral blood cells from the subject, the set comprising expression profiles of a plurality of preterm delivery marker genes from Table 1, to (b) a multimarker classifier, obtained by a comparison of expression levels of the preterm delivery marker genes in a plurality of women who delivered at term to expression levels of the preterm delivery marker genes in a plurality of women who delivered preterm; and
(ii) providing a risk assessment for preterm delivery based on the comparison.

2. The method of claim 1, further comprising obtaining the set of expression profiles prior to the comparing step.

3. The method of claim 2, further comprising obtaining or storing the biological sample prior to determining the set of expression profiles.

4. The method of claim 3, wherein obtaining the biological sample comprises isolating a mononuclear blood cell fraction or lymphocytes from a whole blood sample from the subject.

5. (canceled)

6. The method of claim 1, wherein comprising expression profiles of a plurality of preterm delivery marker genes is accomplished using an assay selected from the group consisting of a sequencing assay, a polymerase chain reaction assay, a hybridization assay, a hybridization assay employing a probe complementary to a mutation, fluorescent in situ hybridization, a nucleic acid array assay, a bead array assay, a primer extension assay, an enzyme mismatch cleavage assay, a branched hybridization assay, a NASBA assay, a molecular beacon assay, a cycling probe assay, a ligase chain reaction assay, an invasive cleavage structure assay, an ARMS assay, and a sandwich hybridization assay.

7. The method of claim 1, further comprising prescribing or providing to the subject a prophylactic therapy for reducing the risk of preterm delivery comprising administering to said subject a progesterone therapy, an anti-inflammatory therapy, an anti-diabetic therapy, therapy to reduce oxidative stress, intravascular hemolysis, endothelial dysfunction or a metabolic alteration associated with a high risk of preterm delivery.

8. (canceled)

9. (canceled)

10. The method of claim 1, wherein the biological sample comprises a cell fraction enriched for mononuclear blood cells or lymphocytes.

11. (canceled)

12. (canceled)

13. The method of claim 1, wherein providing the risk assessment comprises providing a probability score or a preterm delivery risk classification.

14. The method of claim 1, wherein the preterm delivery is spontaneous preterm delivery.

15. The method of claim 14, wherein the spontaneous preterm delivery is very preterm delivery, preterm premature rupture of membrane, moderate preterm delivery, or spontaneous preterm labor/delivery.

16. The method of claim 1, wherein the plurality of preterm delivery marker genes comprises at least five of the preterm delivery marker genes listed in Table 2 or Table 4.

17. (canceled)

18. The method of claim 16, wherein the plurality of preterm delivery marker genes comprises at least ten of the preterm delivery marker genes listed in Table 4.

19. The method of claim 18, wherein the plurality of preterm delivery marker genes comprises the preterm delivery marker genes listed in Table 3 or Table 4.

20. The method of claim 1, wherein the plurality of preterm delivery marker genes comprises at least ten of the preterm delivery marker genes listed in Table 3.

21. The method of claim 20, wherein the plurality of preterm delivery marker genes comprises at least 30 of the preterm delivery marker genes listed in Table 3.

22. (canceled)

23. The method of claim 1, wherein the risk assessment indicates that the subject has a high risk of preterm delivery, further comprising prescribing or providing to the subject a prophylactic therapy for reducing the risk of preterm delivery

24. The method of claim 23, wherein the prophylactic therapy comprises progesterone therapy, anti-inflammatory therapy, or anti-diabetic therapy.

25. (canceled)

26. (canceled)

27. The method of claim 1, wherein the biological sample is obtained antepartum at a gestational age no greater than 20 weeks.

28. The method of claim 27, wherein the biological sample is obtained at a gestational age from about 13 weeks to about 16 weeks.

29. The method of claim 28, wherein the biological sample is obtained within the first trimester of pregnancy.

30. A method of predicting the likelihood of preterm delivery in a subject, comprising:
(i) comparing expression profiles of a plurality of preterm delivery marker genes in a peripheral blood sample from the subject to:
(a) expression profiles of the plurality of preterm delivery marker genes in peripheral blood samples from one or more subjects who delivered at term; or
(b) expression profiles of the plurality of preterm delivery marker genes in blood samples from one or more subjects who delivered preterm; or
(c) both (a) and (b); and
(ii) providing a risk assessment based on the comparison; wherein
the subject has an increased likelihood of preterm delivery if the expression profiles of the plurality of preterm deliver marker genes in the peripheral blood sample from the subject deviate from (a), and wherein the subject does not have an increased likelihood of preterm delivery if the expression profiles of the plurality of preterm delivery marker genes in the peripheral blood sample from the subject deviate from (b), and wherein
the plurality of preterm delivery marker genes comprise five or more genes listed in Table 1.

31. The method of claim 30, further comprising obtaining the gene expression profile prior to the comparing step.

32. The method of claim 31, further comprising obtaining or storing the biological sample prior to determining the set of expression profiles.

33. The method of claim 32, Wherein obtaining the biological sample comprises isolating a mononuclear blood cell fraction or lymphocytes from a whole blood sample from the subject.

34. (canceled)

35. The method of claim 30, wherein the biological sample comprises a cell fraction enriched for mononuclear blood cells or lymphocytes.

36. (canceled)

37. The method of claim 30, wherein the preterm delivery is spontaneous preterm delivery.

38. The method of claim 37, wherein the spontaneous preterm delivery is very preterm delivery, preterm premature rupture of membrane, moderate preterm delivery, or spontaneous preterm labor/delivery.

39. The method of claim 30, wherein comparing expression profiles is accomplished using an assay selected from the group consisting of a sequencing assay, a polymerase chain reaction assay, a hybridization assay, a hybridization assay employing a probe complementary to a mutation, fluorescent in situ hybridization, a nucleic acid array assay, a bead array assay, a primer extension assay, an enzyme mismatch cleavage assay, a branched hybridization assay, a NASBA assay, a molecular beacon assay, a cycling probe assay, a ligase chain reaction assay, an invasive cleavage structure assay, an ARMS assay, and a sandwich hybridization assay.

40. The method of claim 30, further comprising prescribing or providing to the subject a prophylactic therapy for reducing the risk of preterm delivery comprising administering to said subject a progesterone therapy, an anti-inflammatory therapy, an anti-diabetic therapy, therapy to reduce oxidative stress, intravascular hemolysis, endothelial dysfunction or a metabolic alteration associated with a high risk of preterm delivery.

41. (canceled)

42. (canceled)

43. A method for identifying a subject at risk of preterm delivery, comprising determining expression profiles of no more than five to five hundred genes in a biological sample comprising peripheral blood cells from a pregnant subject, wherein at least 20% of the genes are selected from the preterm delivery marker genes listed in Table 1.

44. The method of claim 43, wherein at least 30% of the genes of the genes are selected from the preterm delivery marker genes listed in Table 1 or Table 3.

45. (canceled)

46. The method of claim 44, wherein at least 50% of the genes are selected from the preterm delivery marker genes listed in Table 3.

47. The method of claim 46, wherein at least 90% of the genes are selected from the preterm delivery marker genes listed in Table 3.

48. The method of claim 44, comprising determining the expression profiles of no more than five to one hundred genes in a blood sample.

49. The method of claim 43, comprising determining expression profiles of no more than five to one hundred genes.

50. The method of claim 49, comprising determining expression profiles of no more than five to fifty genes.

51. The method of claim 50, comprising determining expression profiles of no more than five to twenty genes.

52. The method of claim 43, further comprising:
(i) comparing the five to five hundred expression profiles to a multimarker classifier; and
(ii) providing a risk assessment for preterm delivery based on the comparison; wherein the multimarker classifier was obtained by a comparison of expression levels of the preterm delivery marker genes in a plurality of women who delivered at term to expression levels of the preterm delivery marker genes in a plurality of women who delivered preterm.

53. The method of claim 43, wherein the biological sample had been obtained antepartum at a gestational age no greater than 20 weeks.

54. The method of claim 53, wherein the biological sample had been obtained at a gestational age from about 13 weeks to about 16 weeks.

55. The method of claim 43, wherein the biological sample had been obtained within the first trimester of pregnancy.

56. The method of claim 43, wherein the preterm delivery is spontaneous preterm delivery

57. The method of claim 56, wherein the spontaneous preterm delivery is very preterm delivery, preterm premature rupture of membrane, moderate preterm delivery, or spontaneous preterm labor/delivery.

58. The method of claim 43, wherein determining expression profiles is accomplished using an assay selected from the group consisting of a sequencing assay, a polymerase chain reaction assay, a hybridization assay, a hybridization assay employing a probe complementary to a mutation, fluorescent in situ hybridization, a nucleic acid array assay, a bead array assay, a primer extension assay, an enzyme mismatch cleavage assay, a branched hybridization assay, a NASBA assay, a molecular beacon assay, a cycling probe assay, a ligase chain reaction assay, an invasive cleavage structure assay, an ARMS assay, and a sandwich hybridization assay.

59. The method of claim 43, further comprising prescribing or providing to the subject a prophylactic therapy for reducing the risk of preterm delivery comprising administering to said subject a progesterone therapy, an anti-inflammatory therapy, an anti-diabetic therapy, therapy to reduce oxidative stress, intravascular hemolysis, endothelial dysfunction or a metabolic alteration associated with a high risk of preterm delivery

60. (canceled)

61. (canceled)

62. A kit for identifying a subject at risk of preterm delivery, comprising:
(i) a set of nucleic acid probes that hybridize under high stringency conditions to the nucleotide sequences of five to five hundred genes in a biological sample comprising peripheral blood cells from a pregnant subject, wherein at least 20% of the genes are selected from the preterm delivery marker genes listed in Table 1, for determining the expression profiles of said genes; and an insert describing: (a) an expression profile of one or more of the preterm delivery marker genes in blood samples from one or more subjects who delivered at term; (b) an expression profile of one or more preterm delivery marker genes in blood samples from one or more subjects who delivered preterm; or (c) a multimarker classifier, wherein the multimarker classifier was obtained by a comparison of expression levels of the preterm delivery marker genes in a plurality of women who delivered at term to expression levels of the preterm delivery marker genes in a plurality of women who delivered preterm.

63. The kit of claim 62, wherein the set of nucleic acid probes comprise primers for RT-PCR amplification of the mRNAs for the ten to one thousand preterm delivery marker genes.

64. A nucleic acid array comprising nucleic acid probes, that hybridize under high stringency conditions to the nucleotide sequences of no more than five to five hundred genes, wherein at least 20% of the genes are selected from the preterm delivery marker genes listed in Table 1.

65. The nucleic acid array of claim 64, wherein the nucleic acid array is provided as one or more multiwell plates, comprising primers for RT-PCR amplification of the mRNAs for the ten to one thousand preterm delivery marker genes.

66. The nucleic acid array of claim 64, wherein the nucleic acid array is provided as a nucleic acid hybridization microarray.

67. The nucleic acid array of claim 64, wherein at least 30% of the genes of the genes are selected from the preterm delivery marker genes listed in Table 1 or Table 4.

68. (canceled)

69. The nucleic acid array of claim 67, wherein at least 50% of the genes of the genes are selected from the preterm delivery marker genes listed in Table 4.

70. The nucleic acid array of claim 69, wherein at least 90% of the genes of the genes are selected from the preterm delivery marker genes listed in Table 4.

71. The nucleic acid array of claim 64, comprising nucleic acid probes that hybridize under high stringency conditions to the nucleotide sequences of no more than five to one hundred genes.

72. The nucleic acid array of claim 71, comprising nucleic acid probes that hybridize under high stringency conditions to the nucleotide sequences of no more than five to fifty genes.

73. The nucleic acid array of claim 72, comprising nucleic acid probes that hybridize under high stringency conditions to the nucleotide sequences of no more than five to twenty genes.
